(19) United States
(12) Patent Application Publication
(10) Pub. No.: US 2014/0155581 A1
(43) Pub. Date: Jun. 5, 2014

(54) Methods For Making Multimeric Polypeptides

(71) Applicant: MEDIMMUNE, LLC, Gaithersburg, MD (US)

(72) Inventors: Changshou Gao, Gaithersburg, MD (US); Nazzareno Dimasi, Gaithersburg, MD (US)

(21) Appl. No.: 14/126,757

(22) PCT Filed: Jul. 2, 2012
(86) PCT No.: PCT/US2012/045229

§ 371 Date: Feb. 17, 2014

Related U.S. Application Data
(60) Provisional application No. 61/504,741, filed on Jul. 6, 2011.

(51) Int. Cl. C07K 16/46; C07K 16/28

(52) U.S. Cl. CPC C07K 16/468; C07K 16/2863
USPC 530/387.3; 536/23.53; 435/320.1; 435/252.3; 435/69.6; 435/68.1; 435/252.33; 435/328; 435/254.2

(57) ABSTRACT

Detailed herein are contiguous, multimeric, multispecific polypeptides, nucleic acids encoding such polypeptides, and methods for making such polypeptides and nucleic acids.

### 1. REFERENCE TO RELATED APPLICATIONS

This application claims the benefit under 35 U.S.C. §119(e) of U.S. Provisional Application No. 61/504,741 filed Jul. 6, 2011. The priority application is hereby incorporated by reference herein in its entirety for all purposes.

### 2. REFERENCE TO A SEQUENCE LISTING

This application incorporates by reference in its entirety the Sequence Listing submitted with this application as text file entitled “AEMS-105WO1_SL_ST25” created on Jun. 29, 2012 and having a size of 77,824 bytes.

### 3. BACKGROUND OF THE INVENTION

Many diseases are caused by or associated with biomolecules, and it is useful to develop disease treatments, therapies, diagnostic reagents, and research reagents that target biomolecules and, optionally, modulate their activities. Agents, including therapeutic, diagnostic, and research agents, that bind to more than one site may be desired, so that a single molecule can bind either to multiple regions of the same target biomolecule, or else to multiple different targets. Multimeric polypeptides are one example of molecules with this binding capability. Within a single multimeric polypeptide, sites on the polypeptide can be engineered to bind two or more different epitopes. For instance, bispecific (or bifunctional) antibodies are multimeric polypeptides in which each variable region on the Fab fragments binds a distinct epitope.

The construction and use of multimeric polypeptides pose unique challenges. When multimeric polypeptides are constructed as fusion proteins, steric constraints may impose limits on the size or complexity of each individual binding site. On the other hand, if multimeric polypeptides are composed of individual subunits, then the subunits must be joined together, for example by chemical conjugation, cross-linking, and/or protein-protein interactions. One type of multimeric polypeptide, bispecific antibodies, have traditionally been produced by chemically linking fragments of antibodies that possess the desired binding properties, as described in U.S. Pat. No. 7,862,813. This procedure requires generating and/or recovering specific antibody fragments, coupling the fragments to cross-linking agents or other moieties that interact, and linking the heterodimers. Recombinant DNA techniques have also been used, by coexpressing two heavy chain-light chain pairs, where the two chains have different specificities. However, because of the random assortment of immunoglobulin heavy and light chains, this approach produces a potential mixture of different antibody molecules, of which only one has the desired bispecific (or multispecific) structure. Purification of the correct molecule, which is usually done by affinity chromatography steps, is cumbersome, time consuming, and significantly reduces total yield of the correct product.

There remains a need for easy, straightforward assembly of multimeric polypeptides, such as bispecific antibodies. The approach described herein makes use of a single nucleic acid that encodes the subunits of the multimeric polypeptide, as well as linker sequences that interconnect the subunits to make a contiguous molecule. After translation and assembly of the contiguous polypeptide, linker sequences may optionally be cleaved, for example by protease cleavage, resulting in a complex of subunits that is multimeric, but no longer contiguous.

### 4. SUMMARY OF DISCLOSURE

The disclosure provides nucleic molecules and methods that can be used to produce unique multimeric, multispecific polypeptides. The unique polypeptides are referred to herein as iMers (innovative multi-mers) or iMers of the disclosure, and a particular subset of these iMers are termed iMabs (innovative monoclonal antibodies) or iMabs of the disclosure. The examples and figures provide numerous illustrative examples of iMers of the disclosure. Although iMers are multimeric, at least a portion of the iMer is initially produced as a contiguous polypeptide chain such that the functional units of the iMer can readily self assemble. Thus, when initially produced, iMers migrate as a single polypeptide chain when subjected to reducing and/or denaturing conditions. Although these iMers can certainly be used diagnostically, therapeutically, or in a research context in this configuration, the polypeptides may also be treated (e.g., with a protease) to cleave the polypeptides linkers that interconnect the subunits of the iMer. Once treated (e.g., with protease) to cleave the linkers, the polypeptide is no longer a single, contiguous polypeptide chain. However, since proper assembly of the molecule has already occurred, this does not prevent or otherwise undermine use of the iMer in any of a variety of therapeutic, diagnostic or research contexts. In fact, as detailed in the examples provided herein, following protease treatment to remove the polypeptide linkers, the iMer may have certain properties. Moreover, additional functions, binding capabilities, and properties may be added by combining iMers with separate polypeptides. Thus, an iMer may associate with one or more additional polypeptides that were not produced as part of the contiguous multimeric polypeptide. This association may occur before, during, or after the contiguous polypeptide portion of the iMer has been cleaved. In addition, iMers may be combined with other iMers to increase complexity.

In one aspect, the disclosure provides a nucleic acid molecule encoding a contiguous, multimeric polypeptide (e.g., an iMer). For example, the disclosure provides a nucleic acid molecule encoding an iMer comprising at least two subunits, each of which includes at least a functional domain and an interaction domain. This exemplary nucleic acid molecule comprises: a nucleic acid portion comprising a nucleotide sequence that encodes a functional domain (FD1) that binds to a first binding site; a nucleic acid portion comprising a nucleotide sequence that encodes an interaction domain (ID1); a nucleic acid portion comprising a nucleotide sequence that encodes a functional domain (FD2) that binds to a second binding site; and a nucleic acid portion comprising a nucleotide sequence that encodes an interaction domain (ID2). Further, in certain features, such an exemplary nucleic acid molecule further comprises at least one nucleic acid portion comprising a nucleotide sequence encoding a polypeptide linker that includes at least one cleavage site (e.g. a protease cleavage site). Additionally, in this exemplary nucleic acid molecule, ID1 and ID2 are capable of associating with each other, and the encoded iMer (contiguous, multimeric polypeptide) is multispecific.

In another aspect, the disclosure provides expression vectors comprising any of the nucleic acid molecules encoding iMers of the disclosure operably linked to a promoter.

In another aspect, the disclosure provides host cells that comprise any of the vectors of the disclosure, and which host cells express the encoded iMer of the disclosure. Moreover, the disclosure provides methods for producing any of the iMers of the disclosure.

In another aspect, the disclosure provides an iMer (e.g., a multimeric polypeptide). For example, the disclosure provides a polypeptide comprising at least two subunits, each of which includes at least a functional domain (FD) and an interaction domain (ID). In certain features, the polypeptide comprises an amino acid sequence with the formula (without reference to any polypeptide linker sequences):

FD1-ID1-FD2-ID2.

This formula represents only the relative position of the functional domains and interaction domains, and it is contemplated that these portions of the iMer may be interconnected directly to one another or may be interconnected via a linker or other moiety. In this example, the first interaction domain and the second interaction domain are capable of associating with each other, and the iMer is a single polypeptide chain when examined under reducing and/or denaturing conditions. In certain features, the polypeptide further comprises at least one polypeptide linker (or more than one polypeptide linker) which may optionally include at least one protease cleavage site. These one or more polypeptide linkers may be at any position(s) in the formula relative to the FD1, ID1, FD2, and ID2 domains.

The disclosure contemplates all combinations of any of the foregoing aspects and features, as well as combinations with any of the aspects and features set forth in the detailed description and examples.

### 5. BRIEF DESCRIPTION OF DRAWINGS

FIG. 1 is a schematic diagram of a representative monovalent bispecific iMer (innovative multi-mer). This format is more specifically referred to herein as an iMab (innovative monoclonal antibody). The iMab is a bispecific antibody, monovalent for each antigen, in a conventional monoclonal antibody format. The iMab has native CL, CH1, CH2 and CH3 domains. The iMab in this example has two distinct CL domain isotypes, one is of the kappa isotype and one is of the lambda isotype. An iMab can have two distinct light-chain isotypes (kappa-lambda) or can have the same light-chain isotype (kappa-kappa; lambda-lambda). Each Fab arm of the iMab binds to a distinct antigen, antigen-1 and antigen-2, as shown in this figure (or to distinct epitopes occurring on the same antigen—epitope 1 and epitope 2). The iMab may possess native interchain disulphide bridges, at the light and heavy chain and at the hinge region, as shown in this representation.

FIG. 2 is a schematic representation of valence (x-axis) versus molecular weight in kilo Dalton (y-axis) of a conventional monoclonal antibody (Mab), of antibody fragments (Fab, scFv, BiTe), of multispecific antibody (BsAb), of multivalent antibody formats (scFv-Fc, scFv-TNF, scFv-p53, trimeribody) and of the iMab. As shown in this diagram, the iMab improves on current antibody formats in view of the fact that it has the molecular weight of a conventional antibody, but, and contrary to the conventional antibody, the iMab is bispecific with monovalent antigen binding.

FIG. 3 is a schematic diagram of the single-chain domains of a representative iMab. As shown in FIG. 3A, all iMab individual domains (VL1, CL1, VH1, VL2, CL2, VH2, CH1, CH2 and CH3) are contiguously connected by a linear single chain peptide. The functional domains of the iMab (Fab1, Fab2, and Fc) are connected by flexible linkers of variable length. At each end of the linker (N-terminus, C-terminus) a protease specific cleavage sequence is placed. Upon iMab formation, the linkers can be removed by protease cleavage treatment. FIG. 3B is a schematic representation of a fully assembled iMab with intact linkers. The linkers may optionally be subsequently removed by protease treatment. Some extra amino acid sequences could remain upon protease treatment at the C-terminus of light-chain1, N-terminus and C-terminus of heavy-chain1, at the N-terminus and C-terminus of light-chain2, and at the N-terminus of heavy-chain2.

FIG. 4 represents the single-chain domain genes of a representative iMab. This single chain gene is more than 5000 DNA bases. The names and location of the iMab domain genes and linkers are schematically labeled. Specific restriction enzymes, as schematically labeled in this diagram, were engineered at specific locations throughout the iMab gene, in order to facilitate cloning of any given antibody gene sequence.

FIG. 5 lists the DNA coding sequence for constant domains and linkers for a representative iMab (SEQ ID NOs: 29-32). In this example, one light chain is of the kappa isotype and the other is of lambda isotype, however any combination of light chain isotypes is contemplated. The variable domain sequences for light chains and heavy chains are represented by boxed text and may encode any desired variable regions.

FIG. 6 lists the amino acid sequence for constant domains and linkers for the representative iMab (SEQ ID NOs: 33-36). The linking sequences, comprising a Thrombin cleavage site, are underlined. The variable domain sequences for light chains and heavy chains are represented by boxed text and may be any desired combination of variable regions. In this representative iMab sequence, the protease cleavage sequence is LVPRGS (SEQ ID NO: 48), which corresponds to a Thrombin cleavage site. Other protease cleavage sequences can be used in place of Thrombin or in combination with Thrombin. The Fc domains in this representative iMab sequence are native human IgG sequences. Fc mutants for engineered effectors functions, such as improved or decreased ADCC, CDC, and/or improved and/or decreased serum half life can be engineered into the Fc sequence of an iMab. In this example one light chain is of the kappa isotype and the other is of lambda isotype. The linkers between light chain 1 and heavy chain 1, heavy chain 1 and light chain 2, and light chain 2 and heavy chain 2 are also provided as SEQ ID NOs: 49, 50 and 51, respectively.

FIG. 7 lists the amino acid sequences for the constant domains of the light chain (kappa isotype) and heavy chain of the iMab arm 1 and the light chain (lambda isotype) and heavy chain of the iMab arm 2 after protease cleavage (represented by SEQ ID NOs: 37, 38, 39, 40 and 36). The variable domain sequences for light chains and heavy chains are represented by boxed text and may be any desired combination of variable regions. These sequences represent the iMab sequences after protease (Thrombin in this example) cleavage. The underlined sequences represent amino acid sequences that remain after protease (Thrombin in this example) treatment (also represented by SEQ ID NO: 38).

FIG. 8 shows a cartoon representation of an intact iMab and the SDS-PAGE analysis. The iMab, depicted in FIG. 8A, has one Fab arm 1 (anti-EGFR) of the kappa light chain isotype, and the Fab arm 2 (anti-IGF1R) of the lambda light chain isotope and is referred to as iMab-EI. The SDS-PAGE analysis (FIG. 8B) was carried out after protein A purification of the iMab-EI. The SDS-PAGE was run under non-reducing and reducing conditions as schematically labeled. The molecular weight standards, in kilo Dalton, are schematically shown and labeled from top-to-bottom (high-to-low) masses. In addition to the iMab-EI, the SDS-PAGE shows also the two conventional monoclonal antibodies (monospecific, bivalent) that are part of the iMab-EI arms. As clearly shown in the SDS-PAGE, the iMab-EI, in contrast to the two conventional antibodies, runs as a distinct single-chain in non-reducing condition. The two conventional monoclonal antibodies used in the analysis have separate light and heavy chains under reducing conditions. The SDS-PAGE analysis in reducing condition shows also that the iMab-EI has the expected molecular mass. The sample's identity in this SDS-PAGE is schematically labeled on top.

FIG. 9 shows the size-exclusion analysis of the iMab-EI after protein A purification. As shown the iMab-EI has two major conformational peaks. These two peaks are identified as conformational aggregates (see Example 5), which are particular molecular conformations that are potentially due to the linkers. The relative percentage of the two peaks is labeled in the figure. The relative percentage may change by varying the linker length. The respective retention times for the two peaks is schematically labeled in the figure. The molecular weight standards, used to calibrate the size-exclusion chromatography column, are schematically shown on the top of the figure, and the molecular weights reported are in kilo Dalton. The x-axis represents the elution volume of the iMab-EI. The y-axis represents the relative absorbance at 280 nanometers. The iMab-EI with intact linkers is also shown in FIG. 8A.

FIG. 10 shows the antigen binding data analyzed using ELISA assays for the iMab-EI with intact linkers after protein A purification, and for the two conventional antibodies that form the two individual arms of the iMab-EI. In this example, the iMab-EI has two antibody arms, one antibody arm is specific for the EGF receptor (EGFR), and the other antibody arm is specific for the IGF1 receptor (IGF1R). The antibody concentration is plotted on a logarithmic scale. Thus, the iMab-EI has dual specificity for EGFR and IGF1R. The conventional anti-EGFR antibody is of the kappa light chain isotype; whereas the anti-IGF1R arm conventional antibody is of the lambda light chain isotype. This iMab-EI has one Fab arm 1 (anti-EGFR) of the kappa light chain isotype, and the Fab arm 2 (anti-IGF1R) of the lambda light chain isotope (see FIG. 8A). FIG. 10A shows the binding to EGFR and FIG. 10B is the binding to IGF1R. As seen in this figure, the two conventional antibodies are binding specifically to their respective antigens; the anti-EGFR conventional antibody binds to EGFR but not to IGF1R, and the anti-IGF1R conventional antibody binds to IGF1R but not to EGFR. In contrast, the innovative monoclonal antibody iMab-EI is capable of binding both antigens (EGFR and IGF1R). Moreover, the specific binding of iMAb-EI for its two antigens can be detected using secondary antibodies specific for anti-kappa or anti-lambda. Differences in signal are likely due to different valences between the conventional antibodies (monospecific bivalent) and the iMab-EI (bispecific monovalent for each antigen).

FIG. 11 shows the unique ability of the iMab-EI with intact linkers to concurrently bind two distinct antigens, EGFR and IGF1R. FIG. 11A is a schematic representation of the dual ELISA assay. Briefly, EGFR was immobilized on the ELISA plate, the iMab-EI was then added followed by the second antigen (IGF1R). The second antigen has a unique tag that can be used for detection purposes using an anti-tag specific monoclonal antibody. Dual binding signal, as observed in FIG. 11B, is only possible if the iMab-EI (as shown) is capable of concurrently binding the two antigens. The iMab-EI Fab arm specificity is schematically labeled.

FIG. 12 shows the iMAb-EI before and after protease treatment (in this specific example, Thrombin). FIG. 12A is a schematic representation of the protease treatment process, showing the iMab-EI before (conformer I) and after (conformer II) protease treatment. FIG. 12B shows the SDS-PAGE analysis under non-reducing conditions (left side) and reducing conditions (right side). The sample's identity in this SDS-PAGE is schematically labeled on top. The molecular weight standards, in kilo Daltons, are schematically shown and labeled from top-to-bottom (high-to-low) masses. The two conventional antibodies, anti-EGFR and anti-IGF1R, are shown as molecular weight controls. The SDS-PAGE includes also as molecular weight controls a mixture of the two conventional arm antibodies. As shown in the SDS-PAGE, protease treatment results in completely removing the linkers form the iMab-EI. As shown, the intact iMab-EI without protease treatment in reducing condition runs as a distinct single band, whereas treatment with the protease releases the individual components of the iMab-EI (namely, the two heavy chains, heavy chain 1 anti-EGFR arm; and heavy chain 2 anti-IGF1R arm; and the two light chains, the anti-EGFR kappa light chain; and the anti-IGF1R lambda light chain).

FIG. 13 shows (as also reported in FIG. 9) the size-exclusion analysis of the iMab-EI after protein A purification but before (FIG. 13A) and after (FIG. 13B) protease treatment (Thrombin in this example). As shown, protease treatment of the iMAb-EI results in near 100% monomeric and homogeneous peak (FIG. 13B), as determined by size-exclusion chromatography. The monomeric peak for the iMab-EI has the expected molecular weight and retention time. This analysis shows that protease treatment results in completely removing the linkers from the iMab-EI. As also shown in FIG. 9, the non-protease treated iMab-EI has two major conformational peaks. These two peaks are identified as conformational aggregates, which are particular molecular conformations potentially due to the linkers. The conformational aggregate status for intact iMab-EI is supported by the fact that when the linkers are removed the iMab-EI becomes a single monomeric and homogeneous peak in size-exclusion chromatography. If the intact iMab-EI was not a conformational aggregate, then the protease treatment would not result in a monomeric homogeneous peak. The relative percentage of the size-exclusion peaks are schematically labeled in both FIG. 13A and FIG. 13B. The respective retention times for the size-exclusion peaks are schematically labeled in both FIG. 13A and FIG. 13B. The x-axis represents the elution volume. The y-axis represents the relative absorbance at 280 nanometers. The iMab-EI with intact linkers or the iMab-EI with removed linkers are also shown in this figure.

FIG. 14 shows the antigen binding data analyzed using ELISA assays for the iMab-EI with linkers removed and after protein A purification (see FIG. 13B), and for the two conventional antibodies that form the two individual arms of the iMab-EI. In this example, one antibody arm of the iMab-EI is specific for EGFR, and the other antibody arm of the iMab is specific for IGF1R. The conventional anti-EGFR antibody is of the kappa light chain isotype; whereas the anti-IGF1R arm conventional antibody is of the lambda light chain isotype (see FIG. 13B). The iMab-EI has one Fab arm 1 (anti-EGFR) of the kappa light chain isotype, and the Fab arm 2 (anti-IGF1R) of the lambda light chain isotype. The antibody concentration is plotted on a logarithmic scale. FIG. 14A shows the binding to EGFR and FIG. 14B is the binding to IGF1R. As seen in this figure, the two conventional antibodies bind specifically to their receptive antigens. In fact, the anti-EGFR conventional antibody binds to EGFR but not to IGF1R, and the anti-IGF1R conventional antibody binds to IGF1R but not to EGFR. Anti-kappa or anti-lambda have been used as detection secondary antibodies. In contrast, the innovative monoclonal antibody iMab-EI is capable of binding both the EGFR and IGF1R antigens. Moreover, the specific binding of iMab-EI for its two antigens can be detected using secondary antibodies specific for anti-kappa or anti-lambda. Differences in signal are due to different valences between the conventional antibodies (monospecific bivalent) and the iMab-EI (bispecific monovalent for each antigen).

FIG. 15 shows the unique ability of the iMab-EI, following removal of linkers, to concurrently bind two distinct antigens, in this case EGFR and IGF1R, as measured using the dual ELISA binding assay detailed above (see FIG. 11B for the schematic of the dual ELISA assay used). The antibody concentration is plotted on a logarithmic scale. The iMab-EI with removed linkers is shown in FIG. 13B. Briefly, EGFR was immobilized on the ELISA plate, the iMab-EI with removed linkers was then added followed by the second antigen (IGF1R). The second antigen has a unique tag that can be used for detection purposes using an anti-tag specific monoclonal antibody. Dual binding signal is only possible if the iMab-EI is capable of concurrently binding the two antigens. As shown in this figure and in FIG. 11, iMab-EI is able to concurrently bind both antigens with and without linkers.

FIG. 16 shows the size-exclusion analysis (FIG. 16A) of the anti-EGFR antibody (dotted line), the anti-IGF1R antibody (dashed line), and of an equal mixture of the two antibodies (straight line). The insert in FIG. 16A is a zoom of the size-exclusion chromatography of the conventional antibodies and their mixture from 8 to 9.5 minutes of retention time. The retention times for the anti-EGFR and the anti-IGF1R conventional antibodies were 8.5 and 8.9 minutes, respectively. FIG. 16B is the size-exclusion chromatography of the iMab-EI after protein A purification and protease treatment (Thrombin in this example). The retention time for the iMab was 8.3 minutes. The respective retention times for the size-exclusion peaks are schematically labeled in both FIGS. 16A and 16B. The x-axis represents the elution volume. The y-axis represents the relative absorbance at 280 nanometers. These size-exclusion analyses confirm that this iMab-EI has a unique retention time, which is different from the retention time of the anti-EGFR conventional antibody, the anti-IGF1R conventional antibody and the mixture of the two conventional antibodies.

FIG. 17 shows the isoelectric point (pI) analysis of the anti-EGFR antibody (FIG. 17A), the anti-IGF1R antibody (FIG. 17B), and the iMab-EI (FIG. 17C) after protease treatment (Thrombin in this example) and protein A purification. The x-axis represents the pI value. The y-axis represents the relative absorbance at 280 nanometers. The pI standards used (pI 4.22 and pI 9.77) are schematically labeled in FIGS. 17A, 17B, and 17C. These pI analyses confirm that the iMab has a unique pI, 8.43, which is different from the pI of the anti-EGFR and anti-IGF1R conventional antibodies, which have pI values of 7.72 and 8.24, respectively. The pI values are schematically shown for each construct.

FIG. 18 shows the results of Differential Scanning calorimetry (DSC) analysis. FIG. 18A is the DSC denaturation analysis for the anti-EGFR conventional antibody. This analysis showed that the anti-EGFR conventional antibody has two transition temperatures 69° C. and 81° C., respectively. FIG. 18B is the DSC denaturation analysis for the anti-IGF1R conventional antibody. This analysis showed that the anti-IGF1R conventional antibody has two transition temperatures 69° C. and 81° C., respectively. FIG. 18C is the DSC denaturation analysis for the mixture of the anti-EGFR and anti-IGF1R conventional antibodies. This analysis showed that as expected, the mixture of the two conventional antibodies has two transition temperatures 69° C. and 81° C., respectively. FIG. 18D is the DSC denaturation analysis for the iMab-EI that is bispecific with monovalency for EGFR and IGF1R. This analysis showed that the iMab-EI has two transition temperatures 69° C. and 81° C., respectively. The conventional anti-EGFR antibody is of the kappa light chain isotype; whereas the anti-IGF1R conventional antibody is of the lambda light chain isotype. Overall, the DSC analysis showed that the iMab has denaturation transitions that are comparable to the denaturation transitions of the mixture of the two conventional antibodies that form the independent iMab-EI arms.

FIG. 19 shows the results of a depletion assay. FIG. 19A, left side, is a schematic representation of the depletion assay for an iMab-EI, specific for EGFR and IGF1R, when EGFR is coated on an ELISA plate and the well solution is then probed against IGF1R immobilized on the ELISA plate. All antibodies and the iMab-EI are at 0.02 μg/mL in this study. FIG. 19B shows the depletion results of the iMab-EI, specific for EGFR and IGF1R, and of the two parental arm antibodies, anti-EGFR and anti-IGF1R, when preabsorbed on EGFR and the resulting supernatant is probed by ELISA on immobilized IGF1R. As shown in column F, when the iMab-EI is preabsorbed on EGFR, the resulting supernatant shows minimal binding to immobilized IGF1R. This means that the iMab-EI preparation contains only the iMab-EI and not the two parental arm antibody components. Column E shows that, as expected, the non-absorbed iMab-EI is capable of binding to immobilized IGF1R. This assay also shows the expected binding of the anti-IGF1R antibody, both non-absorbed (column C) and preabsorbed on EGFR (column D), to immobilized IGF1R. Columns A and B are the negative control anti-EGFR antibody, non-absorbed (A) and pre-absorbed on EGFR (B), probed by ELISA on immobilized IGF1R. FIG. 19A, right side, is a schematic representation of the depletion assay for an iMab-EI, specific for EGFR and IGF1R, when IGF1R is coated on an ELISA plate and the well solution is then probed against EGFR immobilized on ELISA plate. FIG. 19C shows the depletion results of the iMab-EI, specific for EGFR and IGF1R, of the parental arm antibodies, anti-EGFR and anti-IGF1R, when preabsorbed on IGF1R and the resulting supernatant is probed by ELISA on immobilized EGFR. As shown in column F, when the iMab-EI is preabsorbed on IGF1R, the resulting supernatant shows minimal binding to immobilized EGFR. This means that the iMab-EI preparation contains only the iMab-EI and not the two parental arm antibody components. Column E shows that, as expected, the non-absorbed iMab-EI is capable of binding to immobilized EGFR. Columns C and D are the negative control anti-IGF1R antibody non-absorbed (C) and preabsorbed on IGF1R (D), which as expected do not bind to immobilized EGFR. Columns A and B represent the expected binding of the anti-EGFR antibody, both non-absorbed (A) and pre-absorbed on IGF1R (B), to immobilized EGFR.

FIG. 20 shows in vivo efficacy for iMab-EI, specific for EGFR and IGF1R (diamond). Negative controls include untreated animals (upside-down triangle) and animals treated with an antibody isotype control (circle). The animals were dosed two times weekly for the entire duration of the experiment. This efficacy study shows that the iMab-EI is efficacious in vivo with about 73% tumor growth inhibition. The tumor model used in this study is human primary NSCL (non-small-cell-lung) cancer expressing both EGFR and IGF1R receptors.

FIG. 21 is a schematic diagram of a representative iMer, an iMab in this example, (FIG. 21A) engineered with a linked protease (shown as a circle). Upon folding the protease will undergo auto-cleavage and will also cleave off the iMer linkers as schematically shown in FIG. 21B. The protease cleavage can occur while the iMer is formed, can occur in the intracellular compartment, can occur while the iMer is translocated across the intracellular compartments, can occur while the iMer is secreted in the medium, or can occur in the culture medium after secretion.

FIG. 22 is a schematic diagram of a representative iMer, an iMab in this example, (FIG. 22A) engineered with a protease cleavage site (shown as a filled diamond) that is substrate for a cellular protease. Cellular proteases include, but are not limited to, Furin and the furin class of proteases, Enterokinase, and the enterokinase class of proteases, Thrombin, and the thrombin class of proteases. In addition, the cell expressing the iMer can be engineered to over-express a protease, which may be an endogenous or heterologous protease. Upon iMer assembly, the cellular protease will cleave off the iMer linkers as schematically shown in FIG. 22B. The protease cleavage can occur while the iMer is formed, can occur in the intracellular compartment, can occur while the iMer is translocated across the intracellular compartments, can occur while the iMer is secreted in the medium, or can occur in the culture medium after secretion if the protease is secreted in the culture medium or if the cells are engineered to secrete the protease in the culture medium.

FIG. 23 is a schematic diagram of a representative iMer hybrid, an iMab in this example, which is composed of two distinct Fc domains. For example, but not by way of limitation, one Fc arm could be from one IgG isotype and the other Fc arm from another IgG isotype.

FIG. 24 is a schematic representation of some iMer constructs comprising only a portion of an Fc domain for dimerization that can be engineered using this single-chain-polypeptide-protease-cleavage technology. Domain composition of the illustrated iMers is schematically shown in each cartoon representation. The insert shows the linker and the protease recognition site. The scFv portions in these iMers could be in the VH-Linker-VL or VL-Linker-VH orientation.

FIG. 25 is a schematic representation of a number of possible alternative iMer constructs that can be engineered using this single-chain-polypeptide-protease-cleavage technology. Also represented are iMer constructs that incorporate a second polypeptide chain which is expressed separately and then associates with the polypeptide portions expressed as a single contiguous polypeptide chain, see for example iMer-3n, iMer-4-n, iMer-5n and iMer-6n. Domain composition of the illustrated iMers is schematically shown in each cartoon representation. The insert shows the linker and the protease recognition site. The scFvs in these iMers could be in the VH-Linker-VL or VL-Linker-VH orientation.

FIG. 26 is a schematic representation of iMer constructs (as shown in FIG. 25) that can be engineered using this single-chain-polypeptide-protease-cleavage approach after the linkers have been removed by protease treatment. Domain composition of the illustrated iMabs is schematically shown in each cartoon representation. The scFvs in these iMabs could be in the VH-Linker-VL or VL-Linker-VH orientation.

FIG. 27 is a schematic representation of a bispecific bivalent iMer, also referred to as an iMab-DFD, where DFD stands for Dual Fab Domain. The individual domains of the iMab-DFD are schematically labeled in the figure. The iMab-DFD is composed by two tandem Fab domains connected as a single-chain to the Fc region. The two Fab domains can be of the same specificity or of different specificity. The iMab-DFD depicted is bispecific and bivalent for each antigen. Other multispecificities or multivalences can be engineered into an iMab-DFD. The insert shows the linker and the protease recognition site.

FIG. 28 is a schematic representation of a trispecific bivalent iMer also referred to as an iMab-TFD, where TFD stands for Triple Fab Domain. The individual domains of the iMab-TFD are schematically labeled in the figure. The iMab-TFD is composed by three tandem Fab domains connected as a single-chain to the Fc region. The three Fab domains can be of same specificity or of different specificity. The iMab-TFD depicted is trispecific and bivalent for each antigen. Other multispecificities or multivalences can be engineered into an iMab-TFD. The insert shows the linker and the protease recognition site.

FIG. 29 is a schematic representation of two non-limiting examples of iMer constructs comprising non-Ig dimerization motifs that can be engineered using this single-chain-polypeptide-protease-cleavage technology. Domain composition of the illustrated iMabs is schematically shown in each cartoon representation. The insert shows the linker and the protease recognition site.

FIG. 30 lists the amino acid sequence of an iMer with intact linkers and alternative cleavage sites. Panels A and B show an iMer with intact linkers and a Furin cleavage site, identified by the sequence RKKR (SEQ ID NOs: 26, 41, 42, 43 and 36) or an iMer with intact linkers and a human Enterokinase cleavage site, identified by the sequence GDDDK (SEQ ID NOs: 25, 44, 45, 46 and 36). The linkers between light chain 1 and heavy chain 1, heavy chain 1 and light chain 2, and light chain 2 and heavy chain 2 are underlined and provided as SEQ ID NOs: 52, 53, and 54 for RKKR containing linkers and as SEQ ID NOs: 55, 56, and 57 for the GDDDK containing linkers. The cartoon representation of these iMer is shown in FIG. 22.

FIG. 31 lists the amino acid sequence of an iMer with intact linkers carrying a human Enterokinase cleavage site, identified by the sequence GDDDK (SEQ ID NOs: 44, 45, 46, and 47), and a catalytically active human Enterokinase-light chain-attached via a linker carrying a Enterokinase site (shown in bold and underlined, SEQ ID NO: 58). The cartoon representation of this iMer is shown in FIG. 21.

FIG. 32 show the SDS-PAGE analysis in reducing condition of an iMer targeting EGFR and IGF1R, carrying an Enterokinase recognition sequence, and the human Enterokinase enzyme as shown in FIG. 21 (lane 2); an iMer targeting EGFR and IGF1R, carrying an Enterokinase recognition sequence (lane 3); an anti-EGFR antibody of kappa light chain from which the iMer anti-EGFR arm was derived (lane 4); an anti-IGF1R antibody of lambda light chain from which the iMer anti-IGF1R arm was derived (lane 5); an iMer targeting EGFR and IGF1R, carrying a Thrombin recognition sequence treated with Thrombin (lane 6); and an iMer targeting EGFR and IGF1R, carrying a Thrombin recognition sequence but not-treated with Thrombin (lane 7).

FIG. 33 shows the expression and processing by endogenous Furin of an iMab targeting EGFR and IGF1R (iMab-EI), which carries the Furin cleavage site RKKKR as shown in FIG. 30A. Panel A shows a reducing SDS-PAGE western blot probed with an anti-Furin antibody. Lane 1 is molecular mass standards; lane 2 is 20 microliters of cultured media from CHO cells; lane 3 is approximately 28,000 CHO cells; and lane 4 is purified Furin (0.5 micrograms) obtained form commercial source (R&D System catalog number 1503-SE). Lane 3 clearly shows that CHO express Furin intracellular. Panel B shows the SEC-HPLC profile of CHO expressed iMab-EI with the Furin cleavage sites after protein A purification. 85% of the purified material is fully processed and migrates with the expected retention time of 8.5 minutes. Panel C shows the SEC-HPLC profile of further purified iMab-EI with the Furin cleavage sites, here ˜100% of the material migrates at the expected retention time of 8.5 minutes.

### 6. DETAILED DESCRIPTION

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

The constant region of the heavy chain of IgG may be divided into four smaller domains, CH1, hinge, CH2 and CH3. As used herein “Fc region” and similar terms encompass at least the CH2 and CH3 domain and may further comprise a portion of the hinge region and may include the entire hinge region. It will be understood that the numbering of the Fc amino acid residues is that of the EU index as in Kabat et al. (Ibid). The “EU index as set forth in Kabat” refers to the EU index numbering of the human IgG1 Kabat antibody, while the “Kabat index as set forth in Kabat” refers to the Kabat index numbering of the IgG1 Kabat antibody. Polymorphisms have been observed at a number of immunoglobulin positions (see, e.g., Kimm et al., 2001, J Mol Evol 53:1-9), and thus slight differences between the presented sequence and sequences in the prior art may exist.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., multispecifc antibodies, e.g., PCT publication WO2009018386, incorporated herein by reference in its entirety), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Antibodies also include peptide fusions with antibodies or portions thereof such as a protein fused to an Fc domain. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., Glm(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

The term “multimeric polypeptide” broadly refers to a polypeptide and/or complex of polypeptides of the disclosure. Multimeric polypeptides may be multi-specific, if they bind to more than one target. As described herein, a contiguous multimeric polypeptide is translated as a single, continuous polypeptide, where individual subunits and/or domains may be included on a contiguous polypeptide chain. The term Innovative multi-mers (iMers) is used broadly to refer to multimeric, multispecific polypeptides of the disclosure, wherein at least a portion of the molecule is produced as a contiguous polypeptide chain. iMers may comprise functional domains from immunoglobulin molecules, and this subset of iMers may be referred to as innovative monoclonal antibodies (iMabs). iMabs may be bispecific or may be multi-specific, depending on their configuration. The term iMer is generic and includes iMabs. In other words, iMabs are a category of iMers. Whenever the term iMer is used herein, it is understood to include iMabs and the description is similarly applicable to iMabs. Moreover, even when a particular molecule can be properly characterized as an iMab, such a molecule may also be referred to generically as an iMer. Reference to a molecule as an iMer does not imply that a particular molecule may not also be properly referred to as an iMab. However, it is understood that, in certain features, the term iMer also includes molecules that are not properly referred to as iMabs because, for example, the FDs of the molecule do not correspond to all or a portion of an antigen binding domain of an antibody.

### A. Multimeric Polypeptides

Adding multiple binding and effector functions to a single therapeutic molecule can greatly enhance the capabilities of the molecule. For example, a multimeric, multispecific polypeptide may bind to more than one region of the same target biomolecule, conferring greater specificity than a uni-specific polypeptide that binds to only one region. Alternately, a multimeric polypeptide may bind to multiple target biomolecules, such as targets that are present in a complex, or targets for which sequestering and/or clustering is desired. In a third scenario, the same multimeric polypeptide may perform distinct functions at any given time, depending on the localization and/or expression of its target molecules.

Because of their complexity, multimeric polypeptides are more difficult to assemble than their uni-specific counterparts. Often, multimeric polypeptides must be assembled from individual subunits by chemical cross-linking, conjugation, and/or via protein-protein interactions. This assembly not only adds extra steps to the process of producing the multimeric polypeptide, but may also require specialized engineering of the subunit regions that will be cross-linked, conjugated, or reacted with other subunits. Moreover, the assembly reaction may be inefficient at producing hetero-mers, because reaction products include homo-mers in addition to hetero-mers. For example, assembly of bispecific antibodies from two monomers would be expected to produce a mixture containing 25% of one type of monospecific dimer, 25% of the other type of monospecific dimer, and 50% of bispecific dimers.

To bypass the assembly steps involving individual monomeric subunits, it is useful to produce an entire multimeric polypeptide as single contiguous molecule. Subunits within the contiguous molecules assemble via favorable intramolecular reactions that produce a single multimeric polypeptide. Described herein are contiguous, multimeric polypeptides, also referred to herein as “iMer(s)” comprising domains and linkers suited for intramolecular assembly reactions, nucleic acids encoding these contiguous, multimeric polypeptides, and methods of producing the same. Moreover, the disclosure provides method for cleaving linkers that facilitate assembly of the iMers so that, once the correct molecules have been efficiently made, the iMers may optionally be used in the absence of these linkers.

iMers comprise at least two domains and may further comprise at least one linker, and may comprise additional domains and linkers held together by peptide bonds. The contiguous polypeptide making up the iMer is translated as a single polypeptide chain and remains a single polypeptide chain when subjected to reducing and/or denaturing conditions, such as exposure to agents that disrupt secondary and tertiary structures mediated by hydrogen bonds, salt bridges, disulfide bonds, and non-polar hydrophobic interactions. The iMers of the present disclosure remain as a contiguous polypeptide chain unless and until they are exposed to a cleavage agent (e.g., a protease) that specifically cleaves at a cleavage site engineered into the molecule, for example, within a polypeptide linker region of the molecule. Throughout the application, multimeric polypeptides and iMers are often referred to as contiguous polypeptides. However, the disclosure recognizes that, following optional protease cleavage, the multimeric polypeptide, or iMers of the disclosure, may not form a contiguous polypeptide chain. However, the disclosure contemplates that iMers according to the present disclosure may be readily made and used both prior to and following optional cleavage. In addition, the disclosure recognizes that an iMer may associate with one or more additional polypeptides that were not produced as part of the contiguous multimeric polypeptide. If the iMer comprises cleavage sites, it may associate with the additional polypeptide(s) prior to, during, or after cleavage. As used herein, the term iMer encompasses molecules where at least a portion of the molecule is produced as a contiguous, multimeric polypeptide. However, iMers may also include additional subunits.

In some aspects, an iMer of the present disclosure comprises at least two subunits, each of which includes at least a functional domain (FD) and an interaction domain (ID), wherein the first functional domain (FD 1) binds to a first binding site and the second functional domain (FD2) binds to a second binding site, and further comprises a polypeptide linker, wherein the two interaction domains ID1 and ID2 are capable of associating with each other, and wherein the iMer is multispecific. “FD” or “functional domain” refers to the portion of an iMer that specifically binds to an antigen (or an epitope). The formula of an exemplary, non-limiting molecule is FD1-ID1-FD2-ID2 (note that polypeptide linkers are not specifically depicted in this formula). The polypeptide linker may have at least one cleavage site (e.g., a protease cleavage site). The cleavage sites (e.g., 1, 2, 3, 4, 5, 6, more than 6) may be specifically engineered as part of the polypeptide linker to provide a site for cleavage by a selected protease or chemical agent. The iMer, in its single contiguous polypeptide form, is a single polypeptide chain when examined under reducing and/or denaturing conditions, and may comprise two or three polypeptide portions that are each polypeptide linkers. Each polypeptide linker may be different from other linkers in the iMer, or at least one of the polypeptide linkers may be different. Each polypeptide linker may comprise at least one cleavage site (e.g., a protease cleavage site). However, it is also recognized that the iMer may include some linkers that do and some linkers that do not include a cleavage site (e.g., a protease cleavage site).

1. Multimeric Polypeptides Comprising Immunoglobulin Domains

Functional domains and interaction domains in contiguous multimeric polypeptides such as iMers may be based on immunoglobulin domains. Immunoglobulin molecules such as monoclonal antibodies (mAbs) are widely used as diagnostic and therapeutic agents, and methods for engineering the binding fragments of mAbs are well-known in the art. Monoclonal antibodies, like all immunoglobulin molecules, are made up of peptide subunits. Typically, there are two heavy chain subunits and two light chain subunits. Each heavy chain contains one variable domain (VH) which contributes to antigen binding, and a constant domain (CH) made up of three or four subregions (CH1, CH2, CH3, CH4). The heavy chain subregions, independently or jointly, may be referred to herein generically as a “heavy constant domain,” and abbreviated as “HCD”. Each light chain contains one variable domain (VL) and one constant domain (CL). There are two isotypes of light chain constant domains, kappa (κ) and lambda (20, found in mammals. The light chain constant domain may be referred to herein generically as a “light constant domain,” and abbreviated as “LCD”. Disulfide bonds join each CH1 domain to one CL domain, and join CH2 domains to one another. Five types of heavy chains (α, δ, ε, γ, and μ) are found in different classes of antibodies (IgA, IgD, IgE, IgG, and IgM). Heavy chains have hinge regions which confer structural flexibility and mobility.

mAb fragments containing only select portions of the mAb molecule, such as Fab, F(ab′)2, Fab′, scFv, di-scFv, sdAb fragments, have also been used as diagnostic or therapeutic agents. In addition, specific residues in the variable domains have been altered to improve binding specificity of antibodies and antibody fragments. Other residues not directly involved in antigen binding have been replaced in order to “humanize” regions of the antibodies and reduce immunogenicity of the mAb. Any mAb domains and/or fragments known in the art may be used in the iMers described herein. A subset of iMers is termed “iMabs” (innovative monoclonal antibodies). In particular, one or more functional domains (FDs) of the iMers may comprise Fab and/or scFvs, or variants thereof. Exemplary, non-limiting variants of scFvs include but are not limited to tandem di-scFvs, tandem tri-scFvs, diabodies, and tri(a)bodies. Also contemplated are interaction domains (IDs) comprising Fc regions. In some aspects the Fc regions may be differentially engineered with mutations to: promote and/or maintain heterodimerization (e.g., chimeric mutations, complementary mutations, lock and dock mutations, knob into hole mutations, etc.); alter half-life (e.g., enhance FcRn binding); modulate effector function (e.g., enhance ADCC); and alter stability (e.g., prevent IgG4 arm exchange). In certain aspects, multimeric polypeptides that comprise immunoglobulin domains may include only a portion of a constant region to promote and/or maintain dimerization.

In some aspects, an iMer (such as an iMab) comprises at least two subunits, each of which includes at least a functional domain (FD) and an interaction domain (ID). For example, an iMab may comprise a first antibody light chain comprising a variable domain (VL1) and a light constant domain 1 (LCD1), a first antibody heavy chain comprising a variable domain (VH1) and a heavy constant domain 1 (HCD1), and further comprises a second antibody light chain comprising a variable domain (VL2) and a light constant domain 2 (LCD2), and a second antibody heavy chain comprising a variable domain (VH2) and a heavy constant domain 2 (HCD2). Together, the VL1 domain and VH1 domain correspond to FD1 and bind to the first binding site. Together the VL2 and VH2 domain correspond to FD2 and bind to the second binding site.

In some aspects, the VL1 and VL2 are not the same antibody light chain variable domain. In some aspects, VH1 and VH2 are not the same antibody heavy chain variable domain. Because the binding properties of VL1-VH1 will be distinct from those of VL2-VH2, the iMab formed by association of these domains is bispecific. In some aspects, the LCD1 and the LCD2 are the same, while in still other aspects, the LCD1 and the LCD2 are not the same antibody light constant domain. In further aspects, the HCD1 and HCD2 are the same, while in still other aspects, the HCD1 and the HCD2 are not the same.

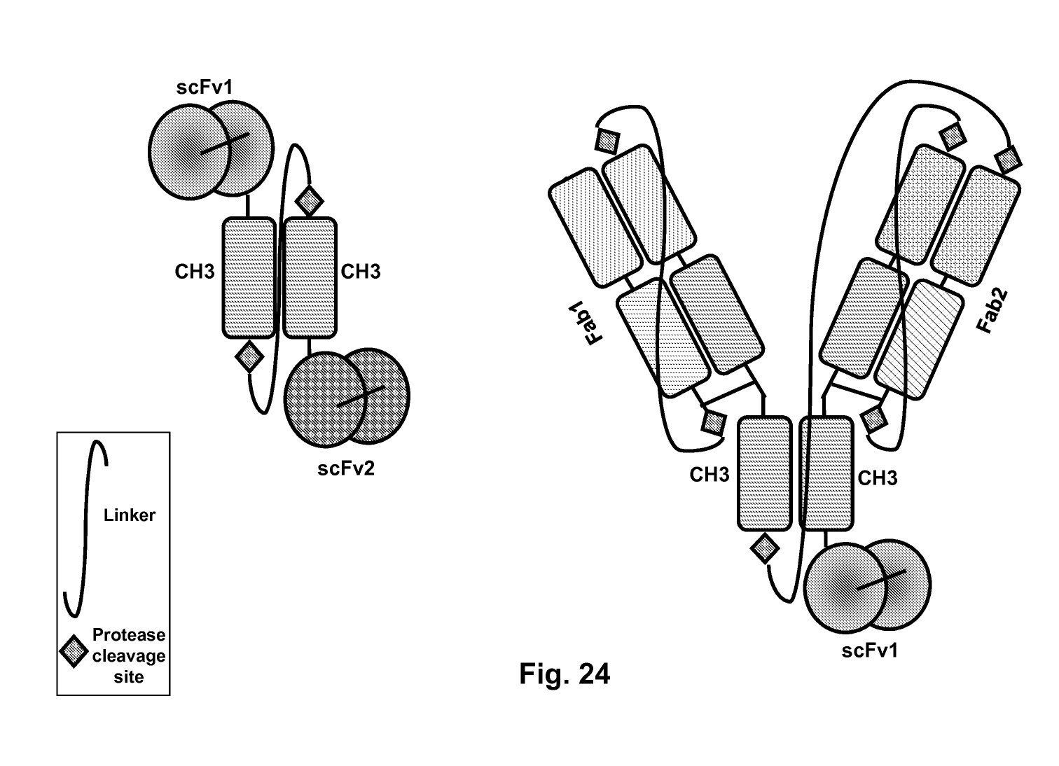

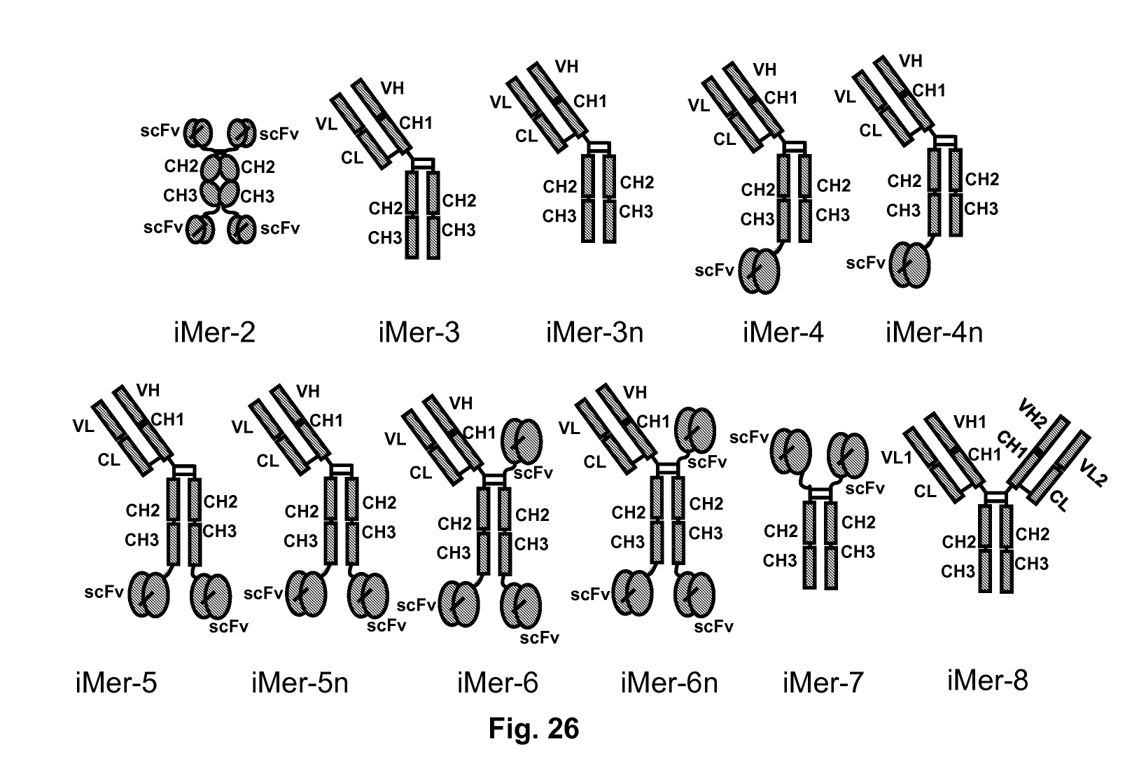

In other aspects, at least one FD is an scFv comprising only the variable regions of an antibody light chain and an antibody heavy chain. The two variable regions may be connected by a short polypeptide linker of, for example, about 25 amino acids, which connects either the N-terminus of the VH to the C-terminus of the VL, or, alternately, the N-terminus of the VL to the C-terminus of the VH. A first scFv may be connected to a first interaction domain, which is, in turn, linked through a polypeptide linker to a second interaction domain that is connected to a second scFv (see, e.g., FIG. 24). Multiple scFvs may be contiguous with the interaction domains. Thus, four, or even more, single scFvs may be part of a single iMer. scFvs can also be combined in iMers with Fab fragments or other types of FDs. Non-limiting configurations are illustrated in FIG. 26. When scFvs are used as a portion of an iMer, it is recognized that the polypeptide linker interconnecting the portions of the scFv optionally is not a cleavable linker.

In certain aspects, an iMer (such as an iMab) comprises a first scFv, a first polypeptide linker, a first interaction domain, a second polypeptide linker, a second interaction domain, a third polypeptide linker, and a second scFv. In certain aspects the first and second scFvs are not the same and confer bispecific binding to the contiguous polypeptide. In other aspects, an iMer comprises a polypeptide sequence comprising an antibody light chain comprising a variable domain (VL1) and a light constant domain 1 (LCD 1), a first polypeptide linker, a polypeptide sequence comprising an antibody heavy chain comprising a variable (VH1) and a heavy constant domain 1 (HCD1), a first interaction domain (ID), a second polypeptide linker, a polypeptide sequence comprising an antibody light chain comprising a variable domain (VL2) and a light constant domain 2 (LCD2), a third polypeptide linker, a polypeptide sequence comprising an antibody heavy chain comprising a variable domain (VH2) and a heavy constant domain 2 (HCD2), a second interaction domain, and a scFv.

Where interaction domains (IDs) in the iMers comprise heavy constant domains (HCDs), the number and composition of these HCDs may be varied. In some aspects, the HCD1 and the HCD2 in the molecule are the same. In other aspects, the HCD1 and the HCD2 are not the same. As noted above, the HCDs may be derived from any class of immunoglobulin molecule. Thus, iMers (including iMabs) may not only bind two separate, distinct antigens, but may also be hybrid molecules whose heavy chains correspond to different immunoglobulin subclasses and/or are differentially modified. Molecules with binding sites for two different antigens may be referred to as trifunctional antibodies because the heavy chains of these antibodies can bind to an Fc receptor. In some aspects, iMers described herein may comprise an Fc portion that binds to Fc receptors and triggers an immune response or other response such as antibody-mediated phagocytosis, antibody-dependent cell-mediated cytotoxicity, or other responses that vary depending on the cells expressing the Fc receptor.

Subregions of HCDs may be present, and the number of subregions may be varied. In some aspects, the HCD1 comprises one or more of a CH1, CH2, and CH3 region, and/or the HCD2 comprises one or more of a CH1, CH2, and CH3 region. For example, the HCD1 may comprise a CH1 and CH2 region, and/or the HCD2 may comprise a CH1 and CH2 region. In other aspects the HCD1 comprises a CH2 and a CH3 region and/or the HCD2 comprises a CH2 region and a CH3 region. In other aspects, the HCD1 comprises a CH1, a CH2, and a CH3 region, and/or the HCD2 comprises a CH1, a CH2, and a CH3 region. Regardless of subregion composition, HCD1 and HCD2 may be the same or different.

Depending on the number and configuration of CH regions in the HCD, one or more hinge regions may be necessary to add flexibility to the structure of the iMers. Hinge regions typically occur between the CH1 and CH2 subregions of the heavy chain of an immunoglobulin molecule, where they allow variability in the angle between the Fab arms of an antibody, rotational flexibility of each individual Fab, and flexibility in the position of the Fab arms relative to the Fc region. However, hinge regions may be engineered between any of the immunoglobulin domains or regions in the iMers described herein. In some aspects, the HCD1 further comprises a hinge region and/or the HCD2 further comprises a hinge region. In other aspects, the HCD1 does not include a hinge region and/or the HCD2 does not include a hinge region.

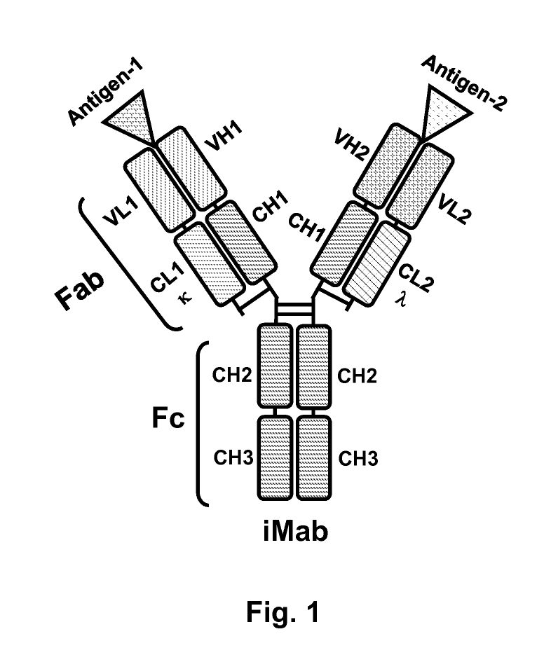

FIG. 1 shows a schematic diagram of a representative contiguous polypeptide called an “iMer” (innovative multimer). The particular sub-class of iMer depicted in FIG. 1 is an iMab. In this example, the iMab is a monovalent bispecific polypeptide. This iMab is bispecific, monovalent for each antigen, and is in a conventional monoclonal antibody format. This iMab has native CL, CH1, CH2 and CH3 domains. The iMab in this example has two distinct CL domain isotypes, one is of the kappa isotype and one is of the lambda isotype. Other exemplary iMabs can have two distinct light-chain isotypes (kappa-lambda) or can have same light-chain isotype (kappa-kappa; lambda-lambda). Each Fab arm of the iMab binds to a distinct antigen, antigen-1 and antigen-2, as shown in this figure. The iMab may possess native interchain disulphide bridges, at the light and heavy chain and at the hinge region, as shown in this representation.

In some cases, bispecific antibodies provide additive and/or synergistic therapeutic effects derived from targeting two antigens simultaneously, with the administration of a single manufactured molecule. For example, a cancer patient having breast carcinoma with moderate expression of HER2, who could not be treated with anti-HER2 mAb therapy, might benefit from the synergic treatment with a bispecific targeting both HER2 and EGFR, provided that the tumor also expresses EGFR. However, treatment with two bivalent mAb or the bivalent bispecific derivative of these two mAbs might pose a severe therapeutic and/or toxic risk. Given that the two mAbs or the bivalent bispecific antibodies react with two receptors that are associated with malignant transformation should increase the tumor specificity of the treatment. However, because the combined mAb treatment or the bivalent bispecific antibody is active against tumor cells with moderate expression of the antigen, some new side effects may arise, due to the presence of some normal tissues with low antigen expression. These tissues may not be sensitive to the single mAb, but may become sensitive to the combined mAb treatment or bivalent bispecific derivative. This potential risk can be more significant with bivalent or multivalent molecules that display enhanced antigen-cell binding due to avidity effects.

In some aspects, the iMer provided herein is bispecific, e.g., a monovalent bispecific antibody (iMab). The iMers described herein provide a superior platform for the generation of bispecific molecules that fulfill all the benefits associated with bispecific antibodies while reducing the potential therapeutic risks mentioned above due to their monovalent nature. Furthermore, the iMers (e.g., iMabs), provided herein are readily expressed, and stable. Accordingly, in certain aspects, an iMer is bispecific comprising two functional domains that specifically bind to two independent antigens (or targets) or two independent epitopes on the same antigen, or two overlapping epitopes on the same antigen. In some aspects, the binding affinities for the two independent antigens are different. In some aspects, the binding affinity for two independent epitopes on the same antigen is about the same. In some aspects, the binding affinity for two independent epitopes on the same antigen is different. In still other aspects, each functional domain has the same specificity (e.g., binds the same, or an overlapping epitope) but binds with a different affinity. In some aspects, the affinities may differ by 3 fold or more. It may be particularly desirable to have one functional domain with higher affinity and one functional domain with lower affinity to prevent the over or under dosing of one of the functional domains. In some aspects, the iMer further comprise additional functional domains that bind a target. The additional functional domains can be specific for one or both target antigens (A and B) of the iMer and/or can be specific for additional target antigens.

In specific embodiments, an iMer is a bispecific antibody (referred herein as an iMab), where each arm can specifically bind to a different target antigen, and for a given pair of different target antigens (A and B), the iMab can bind to one of each. In certain aspects, iMabs can specifically bind to two independent antigens (or targets) or two independent epitopes on the same antigen or two overlapping epitopes on the same antigen. Typically, iMabs will comprise two different variable regions. In some aspects, the binding affinity for the two independent antigens is about the same. In some aspects, the binding affinities for the two independent antigens are different. In some aspects, the binding affinity for two independent epitopes on the same antigen is about the same. In some aspects, the binding affinity for two independent epitopes on the same antigen is different. In still other aspects, each arm has the same specificity (e.g., binds the same, or an overlapping epitope) but binds with a different affinity. In some aspects, the affinities may differ by 3 fold or more. It may be particularly desirable to have one arm with higher affinity and one arm with lower affinity when combining variable regions from antibodies having different in vivo potencies to prevent the over or under dosing of one of the arms.

In certain aspects, an iMab binds the same epitopes or an overlapping epitopes on the same antigen (e.g. a receptor), with different affinities. In particular, the same epitopes or overlapping epitopes, which are in close proximity when the antigen is dimerized. Such an antibody will have a dual characteristic depending on the relative concentration. For example, at high concentration, where the iMab concentration is saturating the antigen concentration, the high affinity binding domain will compete out the low affinity binding domain and little to no avidity effect will take place. That is the antibody will function primarily as a monovalent binding entity and little to no antigen cross-linking/signalling will take place. However, at low concentration avidity effects will come into play and the iMab can concurrently bind both binding sites, preferably on two antigen molecules, leading to antigen cross-linking/signaling. In this manner antigen signaling can be regulated by iMab concentration.

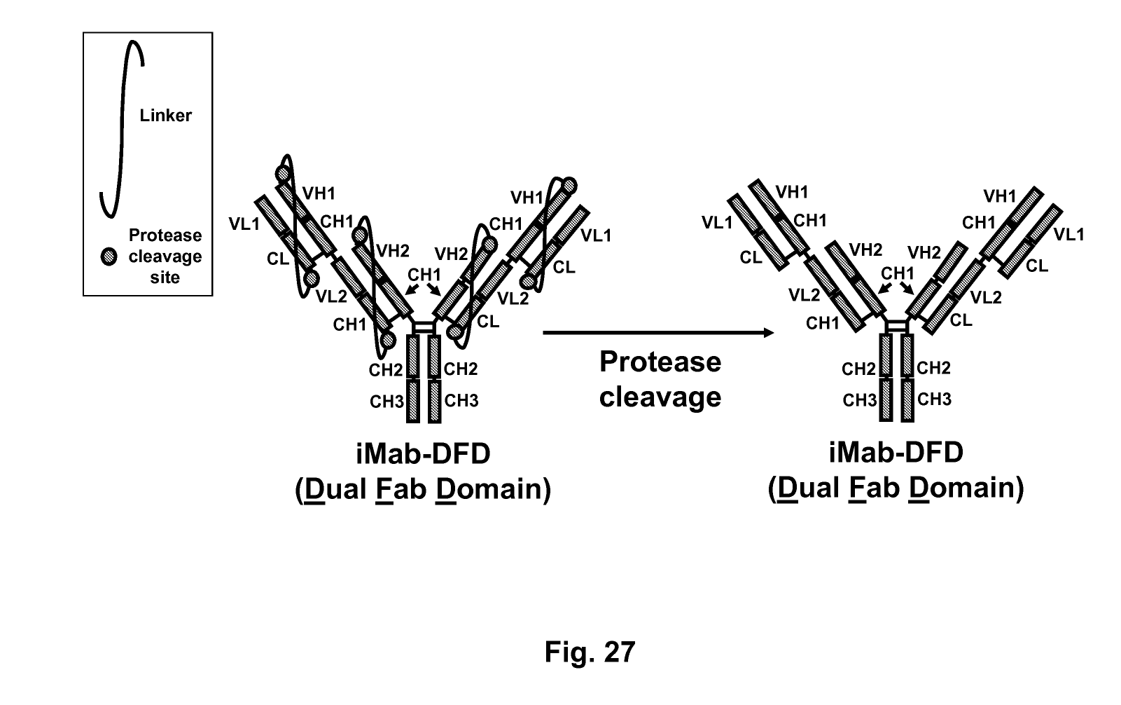

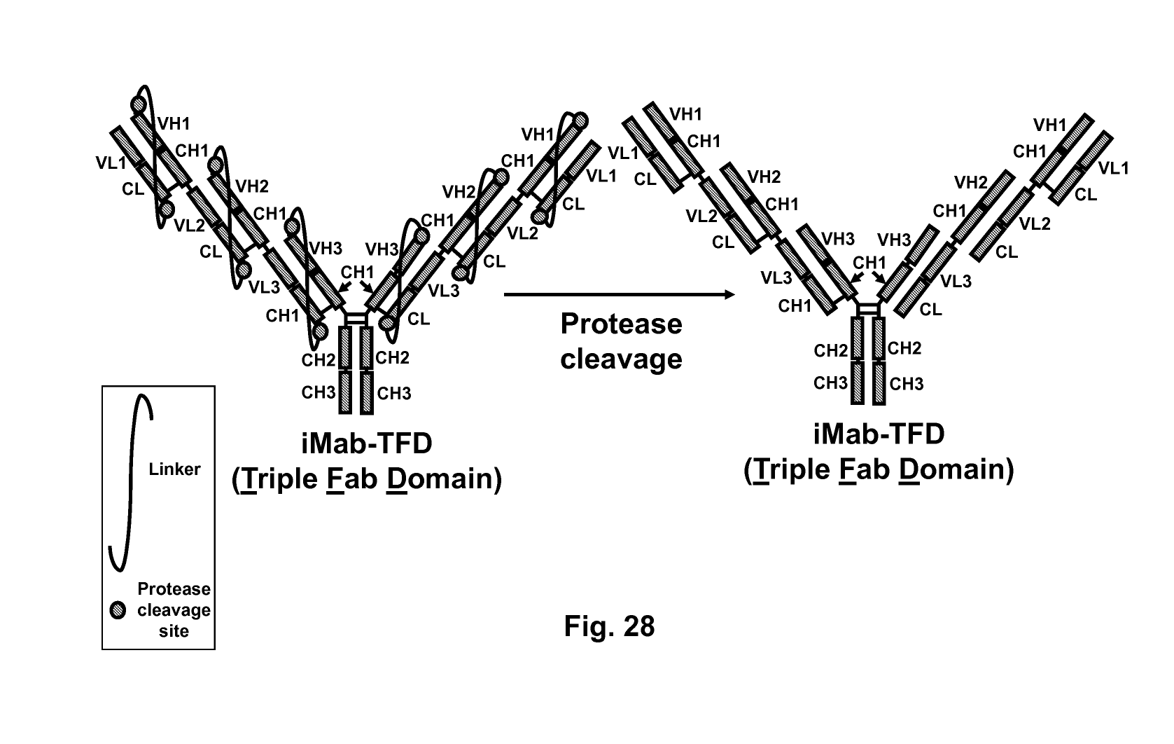

In some aspects, the iMabs further comprise additional binding sites. The additional binding sites can be specific for one or both target antigens (A and B) of the iMab and/or can be specific for additional target antigens. In some aspects, one or more-single chain variable fragments (scFv) are added to the N- or C-terminus of one or both heavy chains and/or one or both light chains, where the one or more scFvs specifically bind to one or more additional target antigens. For example, a monovalent trispecific antibody can be generated by the addition of a scFv (specific for antigen C) to one chain (e.g., heavy or light) of a monovalent bispecific antibody (specific for antigens A and B). In this case, the antibody would be monovalent for antigens A, B, and C. If a scFv (specific for antigen C) is added to two chains (e.g. both heavy chains, both light chains, one heavy chain and one light chain), the trispecific antibody would be monovalent for antigens A and B and bivalent for antigen C. Any possible combination of additional binding sites is contemplated for the monovalent bispecific antibodies herein (see e.g., Dimasi et al. J. Mol. Biol. (2009) 393: 672-692). Alternatively, additional Fab domains may be added to the iMab through the use of cleavable linkers as described herein to generate an iMab-DFD or iMab-TFD (see FIGS. 27 and 28). It is contemplated that the binding affinity of the additional binding sites may be about the same as one or both arms of the iMab or may be different from one or both arms of the iMab. As described above, the relative affinities may be selected or tailored depending on the antigens and the intended use of the molecule.

a. Altered Fc Regions

Altered Fc regions (also referred to herein as “variant Fc regions”) may be used to alter the effector function and/or half life of an iMer of the disclosure (such as an iMab). One or more alterations may be made in the Fc region in order to change functional and/or pharmacokinetic properties of molecules. Such alterations may result in a decrease or increase of Clq binding and complement dependent cytotoxicity (CDC) or of FcγR binding, for IgG, and antibody-dependent cellular cytotoxicity (ADCC), or antibody dependent cell-mediated phagocytosis (ADCP). The present disclosure encompasses iMers (including iMabs) wherein changes have been made to fine tune the effector function, either by enhancing or diminishing function or providing a desired effector function. Accordingly, in one aspect of the disclosure, the iMers comprise a variant Fc region (i.e., Fc regions that have been altered as discussed below). iMers comprising a variant Fc region are also referred to here as “Fc variant iMers.” As used herein “native” refers to the unmodified parental sequence and the iMer comprising a native Fc region is herein referred to as a “native Fc iMer”. Fc variant iMers can be generated by numerous methods well known to one skilled in the art. Non-limiting examples include, isolating antibody coding regions (e.g., from hybridoma) and making one or more desired substitutions in the Fc region. Alternatively, the antigen-binding portion (e.g., variable regions) of an iMer may be subcloned into a vector encoding a variant Fc region. In one aspect, the variant Fc region exhibits a similar level of inducing effector function as compared to the native Fc region. In another aspect, the variant Fc region exhibits a higher induction of effector function as compared to the native Fc. In another aspect, the variant Fc region exhibits lower induction of effector function as compared to the native Fc. Some specific aspects of variant Fc regions are detailed infra. Methods for measuring effector function are well known in the art.

In general, the effector function is modified through changes in the Fc region, including but not limited to, amino acid substitutions, amino acid additions, amino acid deletions and changes in post translational modifications to Fc amino acids (e.g. glycosylation). The methods described below may be used to fine tune the effector function of an iMer of the disclosure, a ratio of the binding properties of the Fc region for the FcR (e.g., affinity and specificity), resulting in an iMer with the desired properties.

It is understood that the Fc region as used herein includes the polypeptides comprising the constant region of an antibody molecule, excluding the first constant region immunoglobulin domain. Thus Fc refers to the last two constant region immunoglobulin domains of IgA, IgD, and IgG, and the last three constant region immunoglobulin domains of IgE and IgM, and, optionally, the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as set forth in Kabat. Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 of IgG1 as numbered by the EU index, and thus slight differences between the presented sequence and sequences in the prior art may exist.

In one aspect, the present disclosure encompasses Fc variant iMers which have altered binding properties for an Fc ligand (e.g., an Fc receptor, Clq) relative to a native Fc iMer. Examples of binding properties include but are not limited to, binding specificity, equilibrium dissociation constant (Kd), dissociation and association rates (koff and kon respectively), binding affinity and/or avidity. It is known in the art that the equilibrium dissociation constant (Kd) is defined as koff/kon. In certain aspects, an iMer comprising an Fc variant region with a low Kd may be more desirable than an iMer with a high Kd. However, in some instances the value of the kon or koff may be more relevant than the value of the Kd. One skilled in the art can determine which kinetic parameter is most important for a given application. For example, a modification that reduces binding to one or more positive regulator (e.g., FcγRIIIA) and/or enhanced binding to an inhibitory Fc receptor (e.g., FcγRIIB) would be suitable for reducing ADCC activity. Accordingly, the ratio of binding affinities (e.g., the ratio of equilibrium dissociation constants (Kd)) for different receptors can indicate if the ADCC activity of an Fc variant iMer of the disclosure is enhanced or decreased. Additionally, a modification that reduces binding to Clq would be suitable for reducing or eliminating CDC activity.

In one aspect, Fc variant iMers exhibit altered binding affinity for one or more Fc receptors including, but not limited to FcRn, FcγRI (CD64) including isoforms FcγRIA, FcγRIB, and FcγRIC; FcγRII (CD32 including isoforms FcγRIIA, FcγRIIB, and FcγRIIC); and FcγRIII (CD16, including isoforms FcγRIIIA and FcγRIIIB) as compared to a native Fc iMer.

In certain aspects, an Fc variant iMer has increased affinity for an Fc ligand. In other aspects, an Fc variant iMer has decreased affinity for an Fc ligand relative to a native Fc iMer.

In a specific aspect, an Fc variant iMer has enhanced binding to the Fc receptor FcγRIIIA. In another specific aspect, an Fc variant iMer has enhanced binding to the Fc receptor FcγRIIB. In a further specific aspect, an Fc variant iMer has enhanced binding to both the Fc receptors FcγRIIIA and FcγRIIB. In certain aspects, Fc variant iMers that have enhanced binding to FcγRIIIA do not have a concomitant increase in binding the FcγRIIB receptor as compared to a native Fc iMer. In a specific aspect, an Fc variant iMer has reduced binding to the Fc receptor FcγRIIIA. In a further specific aspect, an Fc variant iMer has reduced binding to the Fc receptor FcγRIIB. In still another specific aspect, an Fc variant iMer exhibiting altered affinity for FcγRIIIA and/or FcγRIIB has enhanced binding to the Fc receptor FcRn. In yet another specific aspect, an Fc variant iMer exhibiting altered affinity for FcγRIIIA and/or FcγRIIB has altered binding to Clq relative to a native Fc iMer.

In another aspect, Fc variant iMers exhibit increased or decreased affinities to Clq relative to a native Fc iMer. In still another specific aspect, an Fc variant iMer exhibiting altered affinity for Ciq has enhanced binding to the Fc receptor FcRn. In yet another specific aspect, an Fc variant iMer exhibiting altered affinity for Clq has altered binding to FcγRIIIA and/or FcγRIIB relative to a native Fc iMer.

It is well known in the art that antibodies are capable of directing the attack and destruction of targeted antigen through multiple processes collectively known in the art as antibody effector functions. One of these processes, known as “antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are required for such killing. Lysis of the target cell is extracellular, requires direct cell-to-cell contact, and does not involve complement. Another process encompassed by the term effector function is complement dependent cytotoxicity (hereinafter referred to as “CDC”) which refers to a biochemical event of antibody-mediated target cell destruction by the complement system. The complement system is a complex system of proteins found in normal blood plasma that combines with antibodies to destroy pathogenic bacteria and other foreign cells. Still another process encompassed by the term effector function is antibody dependent cell-mediated phagocytosis (ADCP) which refers to a cell-mediated reaction wherein nonspecific cytotoxic cells that express one or more effector ligands recognize bound antibody on a target cell and subsequently cause phagocytosis of the target cell.

It is contemplated that Fc variant iMers are characterized by in vitro functional assays for determining one or more FcγR mediated effector cell functions. In certain aspects, Fc variant iMabs have similar binding properties and effector cell functions in in vivo models (such as those described and disclosed herein) as those in in vitro based assays. However, the present disclosure does not exclude Fc variant iMers that do not exhibit the desired phenotype in in vitro based assays but do exhibit the desired phenotype in vivo.

The serum half-life of proteins comprising Fc regions may be increased by increasing the binding affinity of the Fc region for FcRn. The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body (or other mammal) or a specific compartment thereof, for example, as measured in serum, i.e., circulating half-life, or in other tissues. Half-life may vary from one immunoglobulin or class of immunoglobulin to another. In general, an increase in antibody (or iMer) half-life results in an increase in mean residence time (MRT) in circulation for the iMer administered.

The increase in half-life allows for the reduction in amount of drug given to a patient as well as reducing the frequency of administration. To increase the serum half life of an iMer, one may incorporate a salvage receptor binding epitope into the iMer (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule. Alternatively, iMers of the disclosure with increased half-lives may be generated by modifying amino acid residues identified as involved in the interaction between the Fc and the FcRn receptor (see, for examples, U.S. Pat. Nos. 6,821,505 and 7,083,784; and WO 09/058,492). In addition, the half-life of iMers of the disclosure may be increased by conjugation to PEG or albumin by techniques widely utilized in the art.

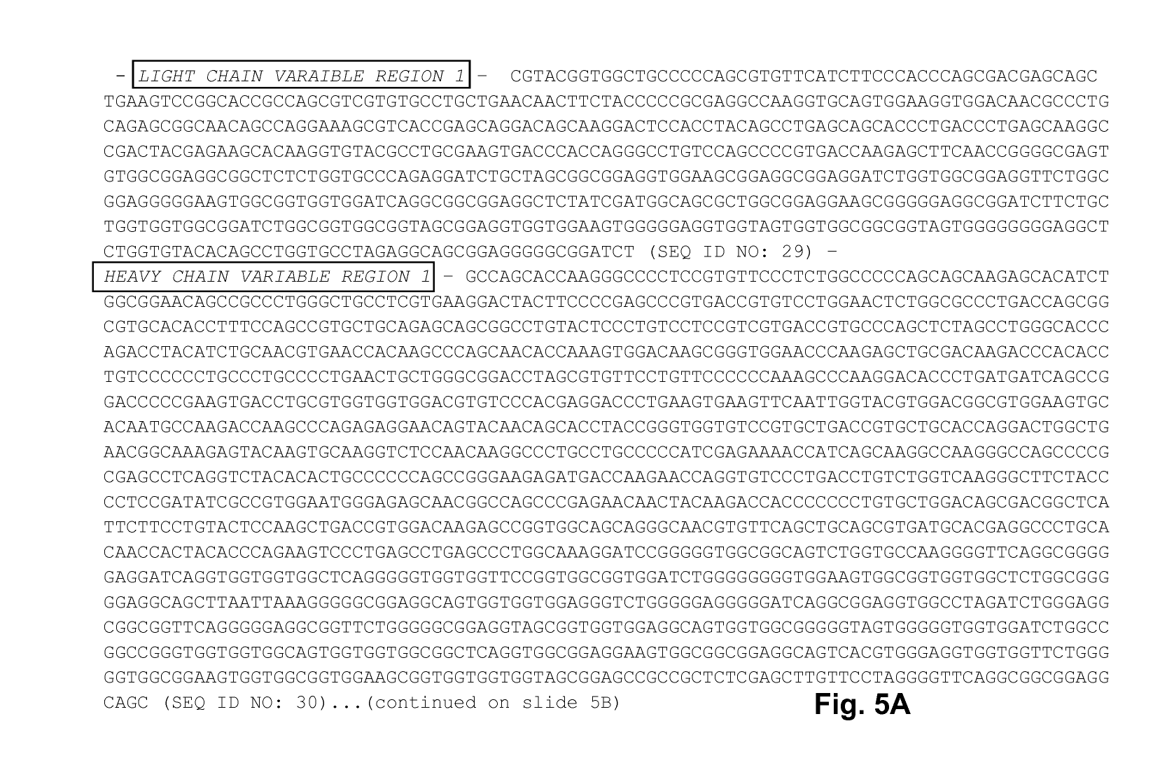

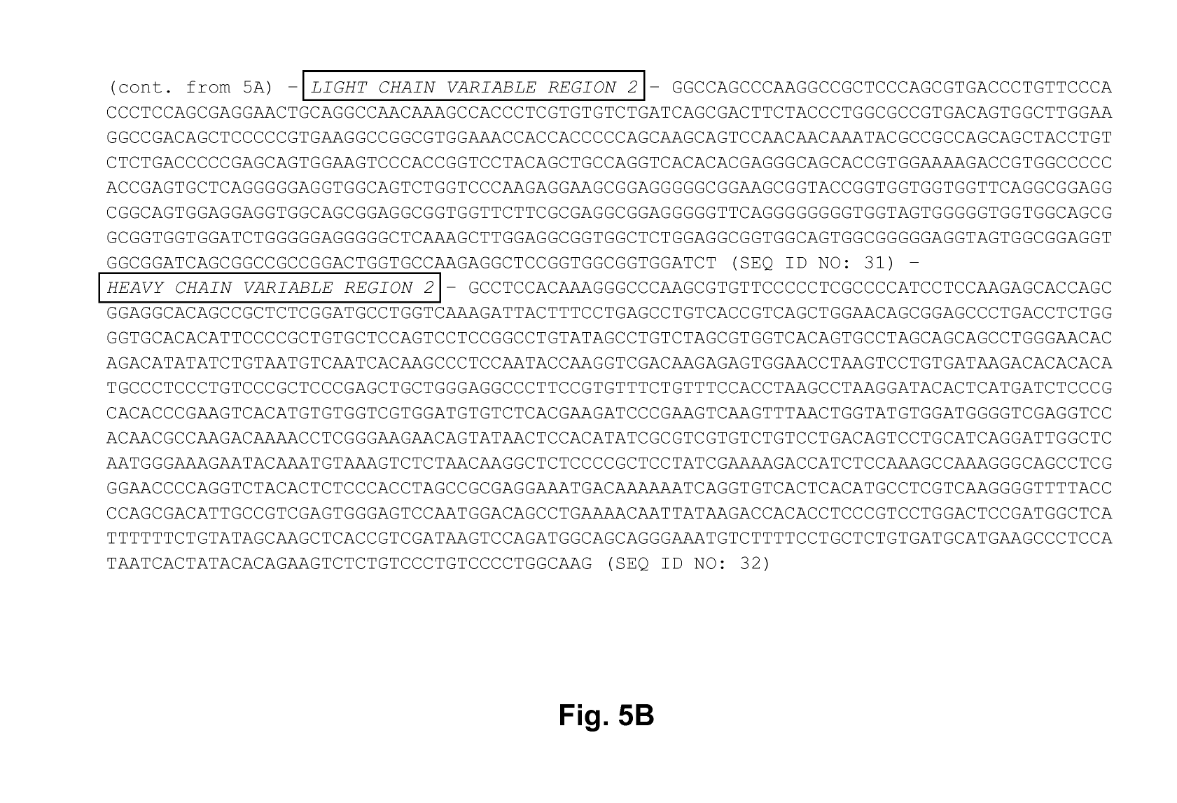

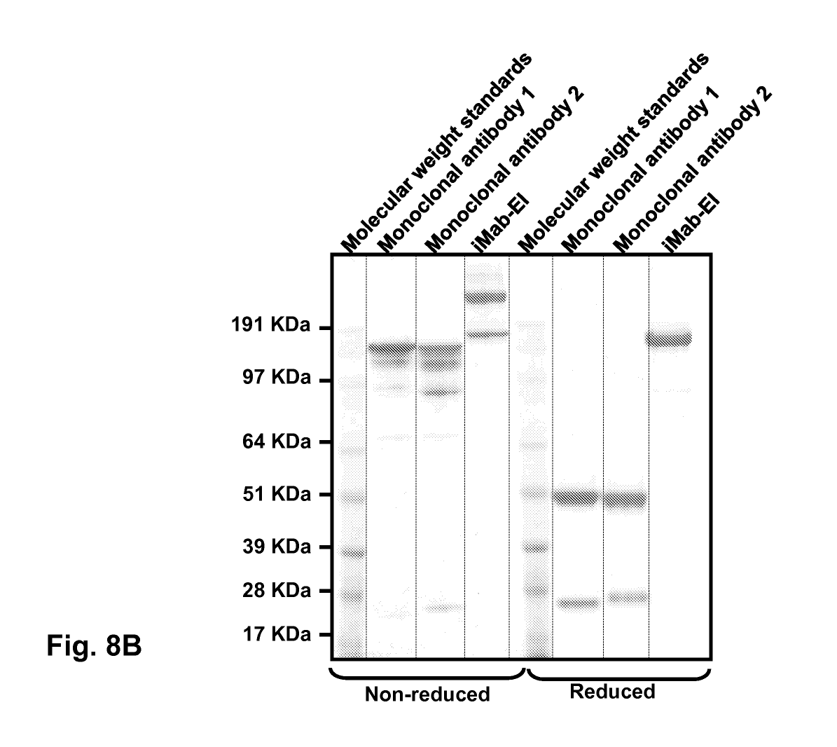

In one aspect, the present disclosure provides Fc variants, wherein the Fc region comprises a modification (e.g., amino acid substitutions, amino acid insertions, amino acid deletions) at one or more positions selected from the group consisting of 221, 225, 228, 234, 235, 236, 237, 238, 239, 240, 241, 243, 244, 245, 247, 250, 251, 252, 254, 255, 256, 257, 262, 263, 264, 265, 266, 267, 268, 269, 279, 280, 284, 292, 296, 297, 298, 299, 305, 308, 313, 316, 318, 320, 322, 325, 326, 327, 328, 329, 330, 331, 332, 333, 334, 339, 341, 343, 370, 373, 378, 392, 416, 419, 421, 428, 433, 434, 435, 436, 440, and 443 as numbered by the EU index as set forth in Kabat. Optionally, the Fc region may comprise a modification at additional and/or alternative positions known to one skilled in the art (see, e.g., U.S. Pat. Nos. 5,624,821; 6,277,375; 6,737,056; 7,083,784; 7,317,091; 7,217,797; 7,276,585; 7,355,008; 2002/0147311; 2004/0002587; 2005/0215768; 2007/0135620; 2007/0224188; 2008/0089892; WO 94/29351; and WO 99/58572). Additional, useful amino acid positions and specific substitutions are exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8-10, 13 and 14 of WO 09/058,492.

In a specific aspect, the present disclosure provides an Fc variant, wherein the Fc region comprises at least one substitution selected from the group consisting of 221K, 221Y, 225E, 225K, 225W, 228P, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 2401, 240A, 240T, 240M, 241W, 241L, 241Y, 241E, 241 R. 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 262I, 262A, 262T, 262E, 263I, 263A, 263T, 263M, 264L, 264I, 264W, 264T, 264R, 264F, 264M, 264Y, 264E, 265A, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F313F, 316D, 318A, 318S, 320A, 320S, 322A, 322S, 325Q, 325L, 325I, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 424F, 434W, 434Y, 436H, 440Y and 443W as numbered by the EU index as set forth in Kabat. Optionally, the Fc region may comprise additional and/or alternative amino acid substitutions known to one skilled in the art including, but not limited to, those exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8, 9 and 10 of WO 09/058,492.

In a specific aspect, the present disclosure provides an Fc variant iMer, wherein the Fc region comprises at least one modification (e.g., amino acid substitutions, amino acid insertions, amino acid deletions) at one or more positions selected from the group consisting of 228, 234, 235 and 331 as numbered by the EU index as set forth in Kabat. In one aspect, the modification is at least one substitution selected from the group consisting of 228P, 234F, 235E, 235F, 235Y, and 331S as numbered by the EU index as set forth in Kabat.

In another specific aspect, the present disclosure provides an Fc variant iMer, wherein the Fc region is an IgG4 Fc region and comprises at least one modification at one or more positions selected from the group consisting of 228 and 235 as numbered by the EU index as set forth in Kabat. In still another specific aspect, the Fc region is an IgG4 Fc region and the non-naturally occurring amino acids are selected from the group consisting of 228P, 235E and 235Y as numbered by the EU index as set forth in Kabat.

In another specific aspect, the present disclosure provides an Fc variant iMer, wherein the Fc region comprises at least one non-naturally occurring amino acid at one or more positions selected from the group consisting of 239, 330 and 332 as numbered by the EU index as set forth in Kabat. In one aspect, the modification is at least one substitution selected from the group consisting of 239D, 330L, 330Y, and 332E as numbered by the EU index as set forth in Kabat.

In a specific aspect, the present disclosure provides an Fc variant iMer, wherein the Fc region comprises at least one non-naturally occurring amino acid at one or more positions selected from the group consisting of 252, 254, and 256 as numbered by the EU index as set forth in Kabat. In one aspect, the modification is at least one substitution selected from the group consisting of 252Y, 254T and 256E as numbered by the EU index as set forth in Kabat. See, U.S. Pat. No. 7,083,784, incorporated herein by reference in its entirety.

In certain aspects, the effector functions elicited by IgG antibodies strongly depend on the carbohydrate moiety linked to the Fc region of the protein (Claudia Ferrara et al., 2006, Biotechnology and Bioengineering 93:851-861). Thus, glycosylation of the Fc region can be modified to increase or decrease effector function (see for examples, Umana et al, 1999, Nat. Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473; U.S. Pat. Nos. 6,602,684; 6,946,292; 7,064,191; 7,214,775;7,393,683; 7,425,446; 7,504,256; U.S. Publication. Nos. 2003/0157108; 2003/0003097; 2009/0010921; POTILLEGENT™ technology (Biowa, Inc. Princeton, N.J.); GLYCOMAB™ glycosylation engineering technology (GLYCART biotechnology AG, Zurich, Switzerland)). Accordingly, in one aspect the Fc regions of iMers of the disclosure comprise altered glycosylation of amino acid residues. In another aspect, the altered glycosylation of the amino acid residues results in lowered effector function. In another aspect, the altered glycosylation of the amino acid residues results in increased effector function. In a specific aspect, the Fc region has reduced fucosylation. In another aspect, the Fc region is afucosylated (see for examples, U.S. Patent Application Publication No. 2005/0226867). In one aspect, these iMers with increased effector function, specifically ADCC, are generated in host cells (e.g., CHO cells, Lemna minor) engineered to produce highly defucosylated polypeptide with over 100-fold higher ADCC compared to polypeptide produced by the parental cells (Mori et al., 2004, Biotechnol Bioeng 88:901-908; Cox et al., 2006, Nat Biotechnol., 24:1591-7).

Addition of sialic acid to the oligosaccharides on IgG molecules can enhance their anti-inflammatory activity and alter their cytotoxicity (Keneko et al., Science, 2006, 313:670-673; Scallon et al., Mol. Immuno. 2007 March; 44(7):1524-34). The studies referenced above demonstrate that IgG molecules with increased sialylation have anti-inflammatory properties whereas IgG molecules with reduced sialylation have increased immunostimulatory properties (e.g., increase ADCC activity). Therefore, an iMer can be modified with an appropriate sialylation profile for a particular application (US Publication No. 2009/0004179 and International Publication No. WO 2007/005786).

In one aspect, the Fc regions of iMers of the disclosure comprise an altered sialylation profile compared to the native Fc region. In one aspect, the Fc regions of iMers of the disclosure comprise an increased sialylation profile compared to the native Fc region. In another aspect, the Fc regions of iMers of the disclosure comprise a decreased sialylation profile compared to the native Fc region.

In one aspect, the Fc variants of the present disclosure may be combined with other known Fc variants such as those disclosed in Ghetie et al., 1997, Nat Biotech. 15:637-40; Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; U.S. Patent publication 2004/0002587; and International Patent publication WO 99/58572. Other modifications and/or substitutions and/or additions and/or deletions of the Fc domain will be readily apparent to one skilled in the art.

b. Glycosylation

In addition to the ability of glycosylation to alter the effector function of polypeptides modified glycosylation in the variable region can alter the affinity of the antibody (or iMab) for a target antigen. In one aspect, the glycosylation pattern in the variable region of the present iMabs is modified. For example, an aglycoslated iMab can be made (i.e., the iMab lacks glycosylation). Glycosylation can be altered to, for example, increase the affinity of the iMab for a target antigen. Such carbohydrate modifications can be accomplished by, for example, altering one or more sites of glycosylation within the iMab sequence. For example, one or more amino acid substitutions can be made that result in elimination of one or more variable region framework glycosylation sites to thereby eliminate glycosylation at that site. Such aglycosylation may increase the affinity of the iMab for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of IgG). Furthermore, aglycosylated iMabs may be produced in bacterial cells which lack the necessary glycosylation machinery.

2. Polypeptide Linkers

Linkers may be used to join domains/regions of iMers into a contiguous molecule. An exemplary, non-limiting linker is a polypeptide chain comprising at least 4 residues that is flexible, hydrophilic and has little or no secondary structure of its own. Linkers of at least 4 amino acids may be used to join domains and/or regions that are positioned near to one another after the molecule has assembled. Longer linkers may be used to join domains and/or regions that are positioned far apart from one another after the molecule has assembled. Thus, linkers may be approximately 4, 8, 12, 16, 20, 24, 28, 32, 36, 40, 44, 48, or 52 residues. Linkers may also be, for example, from about 100-175 residues. When multiple linkers are used to interconnect portion of the molecule, the linkers may be the same or different (e.g., the same or different length and/or amino acid sequence).

The linker(s) facilitate formation of the desired structure, and interactions between the functional domains and interaction domains will not be impaired. Linkers may comprise (Gly-Ser)n residues, with some Glu or Lys residues dispersed throughout to increase solubility. In some aspects, linkers may contain cysteine residues, for example, if dimerization of linkers is used to bring the domains of the iMer into their properly folded configuration. In some aspects, the iMer (such as an iMab) comprises at least two polypeptide linkers that join domains of the polypeptide. In other aspects, the iMer comprises at least three polypeptide linkers.

Linkers may be cleavable linkers, which contain at least one bond that can be selectively cleaved by a cleavage reagent. Linkers may be engineered to contain protease cleavage sites, so that cleavage occurs in the middle of the linker or in at least one end of the linker. For example, thrombin sites may be engineered at each of the two flanking ends of a linker. Depending on the type of linker used, cleavage may also be mediated by agents such as TCEP, TFA, and DTT. Linkers may be designed so that cleavage reagents remove all residues from the linker from the cleavage product. Other exemplary non-limiting linkers include prodrug linkers whose bonds can be selectively cleaved under in vivo conditions, for example, in the presence of endogenous enzymes or other endogenous factors, or simply in aqueous fluids present in the body or in cells of the body. When iMers contain more than one polypeptide linker, each of the linkers may be different, or at least one of the linkers may be different from the others.

In some aspects, at least one of the polypeptide linkers in the iMer comprises at least one protease cleavage site. One or more polypeptide linkers may comprise two protease cleavage sites. Similarly, the iMer may contain three or more polypeptide linkers, each of which comprises at least one protease cleavage site. In certain aspects, each of the three or more polypeptide linkers comprises two protease cleavage sites. Depending on the protease cleavage site, the cleaved form of the iMer may retain one or more amino acid residues derived from the linker. For example, the iMer, in its final cleaved form, may contain 7-9 amino acid residues from the N-terminus and/or the C-terminus of each linker. It is contemplated that the protease cleavage sites may be engineered such that the majority, or in certain features all of the amino acid residues derived from the linker are removed upon protease digestion. Suitable enzymes having specificity for cleavage sites are known in the art and include, but are not limited to, Thrombin, Furin, tobacco etch virus protease, trypsin proteases, SUMO proteases, Human Rhinovirus HRV2C proteases, Factor Xa, enterokinase, V8 protease, α-lytic protease, etc. Cleavage sites for these enzymes are well known in the art. Thus, for example, where factor Xa is to be used to cleave a polypeptide linker, the polypeptide linker is designed to comprise a sequence encoding a factor Xa-sensitive cleavage site, for example, the sequence IEGR (see, for example, Nagai and Thøgersen (1984) Nature 309:810-812, Nagai and Thøgersen (1987) Meth. Enzymol. 153:461-481, and Pryor and Leiting (1997) Protein Expr. Purif. 10(3):309-319, herein incorporated by reference). Where thrombin is to be used to cleave the polypeptide linker, the polypeptide linker can be designed to comprise a sequence encoding a thrombin-sensitive cleavage site, for example the sequence LVPRGS or VIAGR (see, for example, Pryor and Leiting (1997) Protein Expr. Purif. 10(3):309-319, and Hong et al. (1997) Chin. Med. Sci. J. 12(3):143-147). Cleavage sites for TEV protease are known in the art. See, for example, the cleavage sites described in U.S. Pat. No. 5,532,142, herein incorporated by reference in its entirety.

In certain aspects, the cleavage site(s) (i.e., site introduced for chemical or protease cleavage) is selected such that only the cleavable linker(s) are cleaved. In other aspects mutations may be introduced into the iMer to remove undesirable cleavage sites, for example those that would cleave within a functional domain. In some aspects, the protease cleavage site is selected dependent on a protease endogenously expressed by the host cell. In other aspects, the protease cleavage site is selected such that cleavage may occur at the site of action, for example, at a tumor site where a protease associated with the tumor is active.

In some aspects, cleavage sites (e.g., protease cleavage sites) are engineered into polypeptide linkers that connect domains and/or regions of an iMer that are otherwise found in separate polypeptides. For example, in a wild-type antibody, the heavy chain and light chain fragments are separate polypeptides. Accordingly, a linker comprising one or more cleavage sites (e.g., protease cleavage sites) may be engineered into an iMab to separate the domains of the light chain and heavy chains. Similarly, a linker comprising one or more cleavage sites (e.g., protease cleavage sites) may be engineered into iMabs to separate domains that are spaced far apart from one another after the iMab has assembled.

In other aspects, the polypeptide linkers do not contain cleavage sites. For example, scFvs are fusions comprising variable regions of the heavy and light chains of immunoglobulins connected by short linkers of about 10-25 amino acids. iMers comprising scFvs will not have protease sites engineered into the scFv linkers. Similarly, if linkers are connecting a functional domain (FD) to a nearby interaction domain (ID), cleavage sites may not be necessary.

When iMers comprise domains from immunoglobulin molecules, as in the case of iMabs, the polypeptide linkers may interconnect a light chain constant domain 1 (LCD1) to a VH1 domain, a heavy chain constant domain 1 (HCD1) to a VL2 domain, and/or a light constant domain 2 (LCD2) to a VH2 domain. Specifically, a polypeptide linker may interconnect the LCD1 to the VH1 domain and a polypeptide linker may interconnect the HCD 1 to the VL2 domain and a polypeptide linker may interconnect the LCD2 to the VH2 domain. In this molecule, each polypeptide linker may contain at least one cleavage site (e.g., protease cleavage site).

For iMabs whose domains are arranged in a specific orientation, the linkers connect the domains in a specific order. An exemplary iMab comprises antibody light and heavy chain domains in the following orientation from N-terminus to C-terminus: (i) the polypeptide comprising the antibody light chain comprising the variable domain (VL1) and the light constant domain 1 (LCD1); (ii) the polypeptide comprising the antibody heavy chain comprising the variable domain (VH1) and the heavy constant domain 1 (HCD1); (iii) the polypeptide comprising an antibody light chain comprising the variable domain (VL2) and the light constant domain 2 (LCD2); and (iv) the polypeptide comprising the variable domain (VH2) and the heavy constant domain 2 (HCD2). In this molecule, VL1 comprises a first light chain variable domain, and LCD1 comprises a first light chain constant domain. VH1 comprises a first heavy chain variable domain, and HCD 1 comprises a first heavy chain constant domain. Together, the VL1 domain and VH1 domain immunospecifically bind to a first epitope. At the same time, VL2 comprises a second light chain variable domain and LCD2 comprises a second light chain constant domain. VH2 comprises a second heavy chain variable domain and HCD2 comprises a second heavy chain constant domain. Thus, the VL2 domain and the VH2 domain immunospecifically bind to the second epitope.

The polypeptides in iMabs may be operably linked. For example, the polypeptide of (i) may be operably linked to the polypeptide of (ii) by a first polypeptide linker. In some aspects, the polypeptide of (ii) may be operably linked to the polypeptide of (iii) via a second polypeptide linker. Similarly, the polypeptide of (iii) may be operably linked to the polypeptide of (iv) via a third polypeptide linker. Accordingly, a polypeptide with linkers may include a polypeptide linker that interconnects the light constant domain 1 (LCD1) to the VH1 domain, and/or a polypeptide linker that interconnects the heavy constant domain 1 (HCD 1) to the VL2 domain, and/or a polypeptide linker that interconnects the light constant domain 2 (LCD2) to the VH2 domain. Specifically, the polypeptide may comprise a linker that interconnects the light constant domain 1 (LCD1) to the VH1 domain, a polypeptide linker that interconnects the heavy constant domain 1 (HCD 1) to the VL2 domain, and a polypeptide linker that interconnects the light constant domain 2 (LCD2) to the VH2 domain.

Any or all of the polypeptide linkers in the exemplary, non-limiting iMers may comprise at least one cleavage site (e.g., protease cleavage site). A cleavage site may be located within the linker and/or may be located at one or both ends of the linker sequence. Thus, the first polypeptide linker may comprise two cleavage sites, the second polypeptide linker may comprise two cleavage sites, and/or the third polypeptide linker may comprise two cleavage sites. Each polypeptide linker may contain the same cleavage site sequences, or the cleavage sites may be different (e.g., different protease cleavage sites). Finally, each of the polypeptide linkers may be different, or at least one of the polypeptide linkers may be different from the others. Moreover, when multiple polypeptide linkers are present, it is understood that some linkers may include one or more cleavage site and some linkers may include no cleavage sites (e.g., non-cleavable linkers).

To illustrate, for an iMab which comprises a VL1, a light constant domain 1 (LCD1), a VH1, a heavy constant domain 1 (HCD1), a VL2, a light constant domain 2, a VH2, and a heavy constant domain 2 (HCD2), the polypeptide linker that interconnects the LCD1 to the VH1 domain may include at least one cleavage site, and the polypeptide linker that interconnects the HCD1 to the VL2 domain may include at least one cleavage site, and the polypeptide linker that interconnects the light LCD2 to the VH2 domain may include at least one cleavage site. In some aspects, polypeptide linker that interconnects the LCD 1 to the VH1 domain includes 2 cleavage sites, and the polypeptide linker that interconnects the HCD1 to the VL2 domain includes 2 cleavage sites, and the polypeptide linker that interconnects the LCD2 to the VH2 domain includes 2 cleavage sites. Each of the polypeptide linkers may be the same, or they may be different. In certain aspects the cleavage sites are protease cleavage sites.

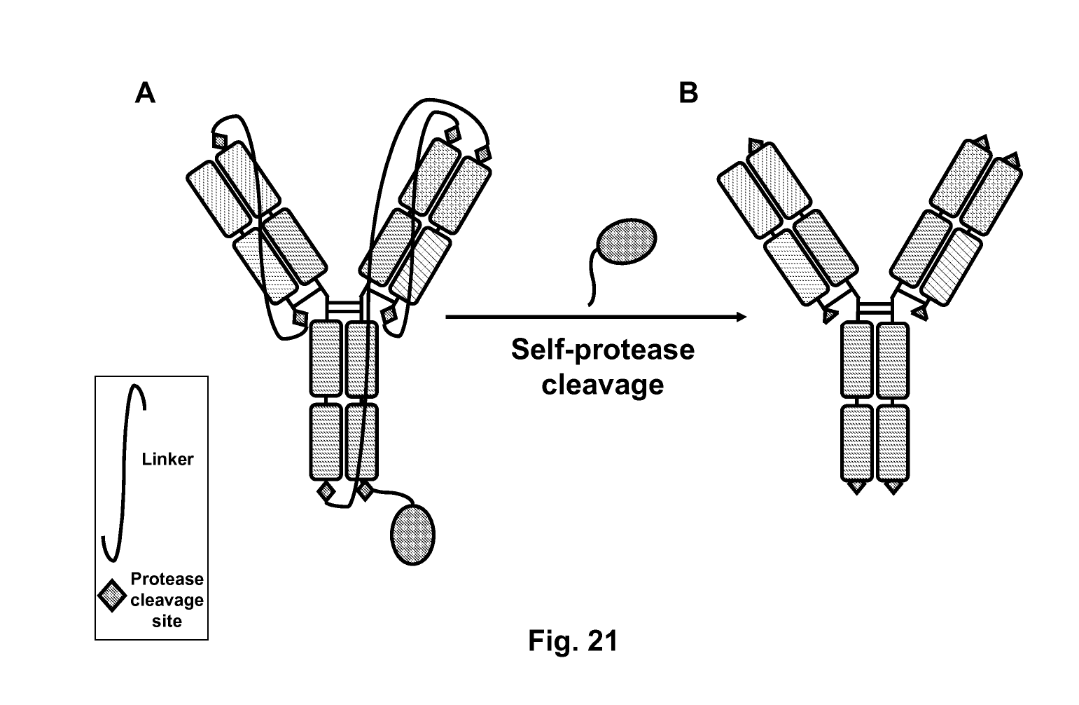

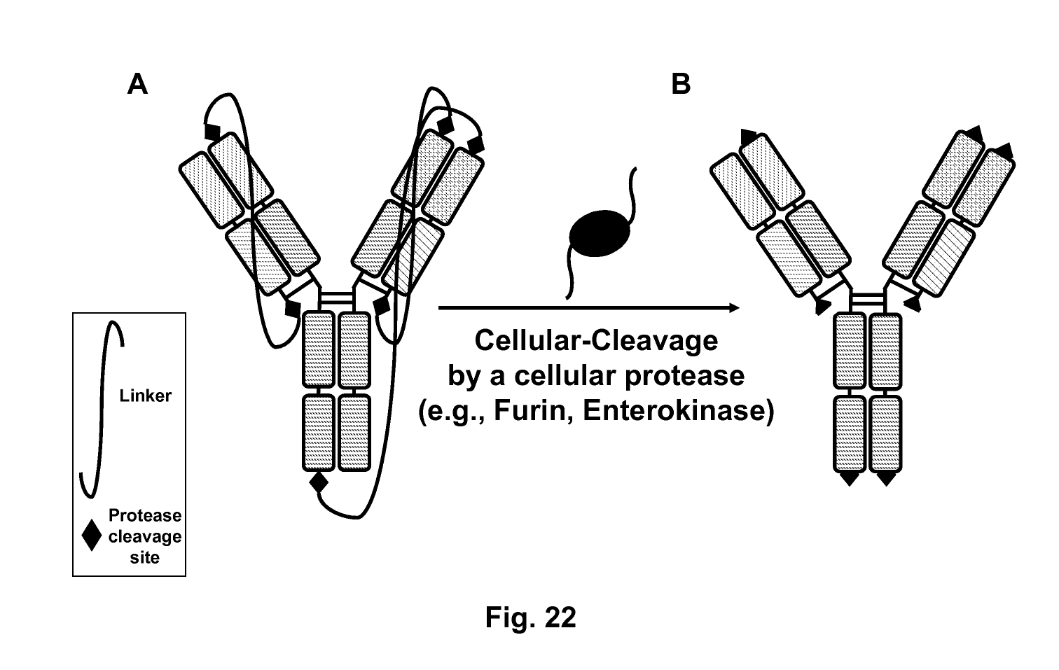

In some aspects, an iMer may comprise an additional polypeptide linker at the C-terminus of the molecule and a polypeptide sequence comprising a protease. In this configuration, the protease mediates self-cleavage of the iMer at any protease cleavage sites in linkers positioned in the iMer (FIG. 21). Alternately, cleavage of protease cleavage sites in the linkers may be mediated by cellular proteases, such as furin (FIG. 22). The relevant protease may be endogenously produced by the cell type in which the iMer is produced or may be exogenously added to the cell culture or protein preparation at any stage in protein production.

a. Configurations of iMabs

Numerous iMab configurations are possible. Non-limiting examples of iMab are provided below. In addition to the domain and linkers specifically provided for, these molecules may have additional domains or linkers at the N-terminus, C-terminus or interspersed in the molecule.

The polypeptide sequence of an iMab may comprise an antibody light chain comprising a variable domain (VL1) and a light constant domain 1 (LCD1), a polypeptide sequence comprising a first polypeptide linker, a polypeptide sequence comprising an antibody heavy chain comprising a variable domain (VH1) and a heavy constant domain 1 (HCD1), a polypeptide sequence comprising a second polypeptide linker, a polypeptide sequence comprising an antibody light chain comprising a variable domain (VL2) and a light constant domain 2 (LCD2), a polypeptide sequence comprising a third polypeptide linker, and a polypeptide sequence comprising an antibody heavy chain comprising a variable domain (VH2) and a heavy constant domain 2 (HCD2), wherein the contiguous polypeptide is a multispecific polypeptide. Thus, an iMab may comprise polypeptide sequences in the following orientation from N-terminus to C-terminus: (i) the polypeptide sequence comprising an antibody light chain comprising VL1 and LCD1, (ii) a polypeptide sequence comprising a first polypeptide linker, (iii) the polypeptide sequence comprising an antibody heavy chain comprising VH1 and HCD1, (iv) a polypeptide sequence comprising a second polypeptide linker, (v) the polypeptide sequence comprising an antibody light chain comprising VL2 and LCD2, (vi) a polypeptide sequence comprising a third polypeptide linker, and (vii) the polypeptide sequence comprising an antibody heavy chain comprising VH2 and HCD2. In certain aspects each linker comprises at least one protease cleavage site.

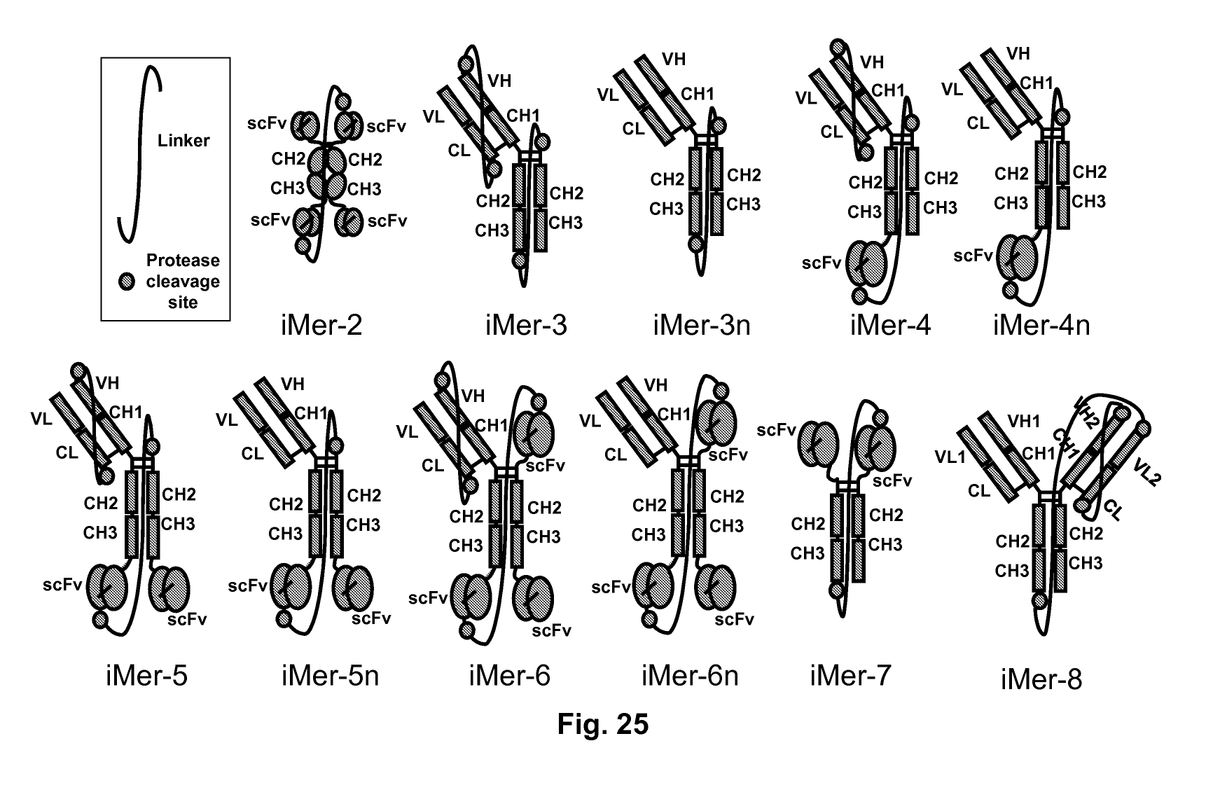

Another iMab comprises a polypeptide sequence comprising an antibody light chain comprising a variable domain (VL1) and a light constant domain 1 (LCD 1), a polypeptide sequence comprising a first polypeptide linker, a polypeptide sequence comprising an antibody heavy chain comprising a variable domain (VH1) and a heavy constant domain 1 (HCD1), a polypeptide sequence comprising a second polypeptide linker, a polypeptide sequence comprising a heavy constant domain 2 (HCD2), wherein the contiguous polypeptide binds one or more epitopes. In some aspects, this iMab comprises polypeptide sequences in the following orientation from N-terminus to C-terminus: (i) a polypeptide sequence comprising an antibody light chain comprising VL1 and LCD1, (ii) a polypeptide sequence comprising a first polypeptide linker, (iii) a polypeptide sequence comprising an antibody heavy chain comprising VH1 and HCD1, (iv) a polypeptide sequence comprising a second polypeptide linker, and (v) a polypeptide sequence comprising HCD2. A non-limiting example of this iMab is illustrated as iMer-3 in FIG. 25. In another aspect, a polypeptide sequence comprising an antibody light chain comprising a VL1 and an LCD1 is expressed separately from a single, contiguous polypeptide comprising, in the following orientation from N-terminus to C-terminus, (i) an antibody heavy chain comprising a VH1 and a HCD 1, (ii) a polypeptide sequence comprising a polypeptide linker, and (iii) a polypeptide sequence comprising a HCD2. The separately-expressed polypeptide sequences are assembled into a single iMab. A non-limiting example of this iMab is illustrated as iMer-3n in FIG. 25. In certain aspects each polypeptide linker comprises at least one protease cleavage site.

Still another iMab comprises a polypeptide sequence comprising polypeptide sequences in the following orientation from N-terminus to C-terminus: (i) a polypeptide sequence comprising an scFv, (ii) a polypeptide sequence comprising a first polypeptide linker, (iii) a polypeptide sequence comprising a heavy constant domain 1 (HCD1), (iv) a polypeptide comprising a second polypeptide linker, (v) a polypeptide sequence comprising a second scFv, (vi) a polypeptide sequence comprising a third polypeptide linker, (vii) a polypeptide sequence comprising a third scFv, (viii) a polypeptide sequence comprising a fourth polypeptide linker, (ix) a polypeptide sequence comprising g a heavy constant domain 2 (HCD2), (x) a polypeptide sequence comprising a fifth polypeptide linker, and (xi) a polypeptide sequence comprising a fourth scFv. The HCD1 and HCD2 may comprise CH2 and CH3 domains. A non-limiting example of this iMab is illustrated as iMer-2 in FIG. 25. In certain aspects, the third polypeptide linker comprises at least one protease cleavage site.

A further iMab comprises a polypeptide sequence comprising polypeptide sequences in the following orientation from N-terminus to C-terminus: (i) a polypeptide sequence comprising an antibody light chain comprising a variable domain (VL1) and a light constant domain (LCD 1), (ii) a polypeptide sequence comprising a first polypeptide linker, (iii) a polypeptide sequence comprising an antibody heavy chain comprising a variable domain (VH1) and a heavy constant domain 1 (HCD1), (iv) a polypeptide sequence comprising a second polypeptide linker, (v) a polypeptide sequence comprising an scFv, (vi) a polypeptide sequence comprising a third polypeptide linker, and (vii) a polypeptide sequence comprising a heavy constant domain 2 (HCD2). A non-limiting example of this Mab is illustrated as iMer-4 in FIG. 25. In certain aspects, the first and second polypeptide linkers each comprise at least one protease cleavage site. In a similar aspect, a polypeptide sequence comprising an antibody light chain comprising a VL1 and an LCD1 is expressed separately from a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising an scFv, (iv) a polypeptide sequence comprising a second polypeptide linker, and (v) a polypeptide sequence comprising a heavy constant domain 2 (HCD2). The separately-expressed polypeptide sequences are assembled into a single iMab. A non-limiting example of this iMab is illustrated as iMer-4-n in FIG. 25. In certain aspects, the first polypeptide linker comprises at least one protease cleavage site.

Yet another iMab comprises a polypeptide sequence comprising polypeptide sequences in the following orientation from N-terminus to C-terminus: (i) a polypeptide sequence comprising an antibody light chain comprising a variable domain (VL1) and a light constant domain (LCD 1), (ii) a polypeptide sequence comprising a first polypeptide linker, (iii) a polypeptide sequence comprising an antibody heavy chain comprising a variable domain (VH1) and a heavy constant domain 1 (HCD1), (iv) a polypeptide sequence comprising a second polypeptide linker, (v) a polypeptide sequence comprising a first scFv, (vi) a polypeptide sequence comprising a third polypeptide linker, (vii) a polypeptide sequence comprising a heavy constant domain 2 (HCD2), (viii) a polypeptide sequence comprising a fourth polypeptide linker, and (ix) a polypeptide sequence comprising a second scFv. A non-limiting example of this Mab is illustrated as iMer-5 in FIG. 25. In certain aspects, the first and third polypeptide linkers each comprise at least one protease cleavage site. In a related aspect, a polypeptide sequence comprising an antibody light chain comprising a VL1 and an LCD1 is expressed separately from a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising a first scFv, (iv) a polypeptide sequence comprising a second polypeptide linker, (v) a polypeptide sequence comprising a heavy constant domain 2 (HCD2), (vi) a polypeptide sequence comprising a third polypeptide linker, and (vii) a polypeptide sequence comprising a second scFv. The separately-expressed polypeptide sequences are assembled into a single iMab. A non-limiting example of this iMab is illustrated as iMer-5n in FIG. 25. In certain aspects, the second polypeptide linker comprises at least one protease cleavage site.

Another iMab comprises a polypeptide sequence comprising polypeptide sequences in the following orientation from N-terminus to C-terminus: (i) a polypeptide sequence comprising an antibody light chain comprising a variable domain (VL1) and a light constant domain (LCD1), (ii) a polypeptide sequence comprising a first polypeptide linker, (iii) a polypeptide sequence comprising an antibody heavy chain comprising a variable domain (VH1) and a heavy constant domain 1 (HCD1), (iv) a polypeptide sequence comprising a second polypeptide linker, (v) a polypeptide sequence comprising a first scFv, (vi) a polypeptide sequence comprising a third polypeptide linker, (vii) a polypeptide sequence comprising a second scFv, (viii) a polypeptide sequence comprising a fourth polypeptide linker, (ix) a polypeptide sequence comprising a heavy constant domain 2 (HCD2), (x) a polypeptide sequence comprising a fifth polypeptide linker, and (xi) a polypeptide sequence comprising a third scFv. A non-limiting example of this Mab is illustrated as iMer-6 in FIG. 25. In certain aspects, the first and third polypeptide linkers each comprise at least one protease cleavage site. In another aspect, a polypeptide sequence comprising an antibody light chain comprising a VL1 and an LCD1 is expressed separately from a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising an scFv, (iv) a polypeptide sequence comprising a second polypeptide linker, (v) a polypeptide sequence comprising a second scFv, (vi) a polypeptide sequence comprising a third polypeptide linker, (vii) a polypeptide sequence comprising a heavy constant domain 2 (HCD2), (viii) a polypeptide sequence comprising a fourth polypeptide linker, and (ix) a polypeptide sequence comprising a third scFv. The separately-expressed polypeptide sequences are assembled into a single iMab. A non-limiting example of this iMab is illustrated as iMer-6n in FIG. 25. In certain aspects, the second polypeptide linker comprises at least one protease cleavage site.

A further iMab comprises a polypeptide sequence comprising polypeptide sequences in the following orientation from N-terminus to C-terminus: (i) a polypeptide sequence comprising a first scFv, (ii) a polypeptide sequence comprising a first polypeptide linker, (iii) a polypeptide sequence comprising a heavy constant domain 1 (HCD1), (iv) a polypeptide sequence comprising a second polypeptide linker, (v) a polypeptide sequence comprising a second scFv, (vi) a polypeptide sequence comprising a third polypeptide linker, and (vii) a polypeptide sequence comprising a heavy chain constant domain 2 (HCD2). A non-limiting example of this Mab is illustrated as iMer-7 in FIG. 25. In certain aspects, the second polypeptide linker comprises at least one protease cleavage site.

Still another iMab comprises a polypeptide sequence comprising polypeptide sequences in the following orientation from N-terminus to C-terminus: (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising an antibody light chain comprising a variable domain (VL2) and a light constant domain 2 (LCD2), (iv) a polypeptide sequence comprising a second polypeptide linker, and (iv) a polypeptide sequence comprising an antibody heavy chain comprising a variable domain (VH2) and a heavy constant domain 2 (HCD2). This polypeptide is expressed separately from a polypeptide sequence comprising an antibody light chain comprising a variable domain (VL1) and a light chain constant domain (LCD1). The two polypeptides assemble into a single iMab, as illustrated in a non-limiting example in iMer-8 in FIG. 25. In certain aspects, the first and second polypeptide linkers each comprise at least one protease cleavage site.

In all of the foregoing iMers and iMabs, the scFv regions, when present, may be in either the VH-Linker-VL or the VL-Linker-VH orientation.

3. Multimeric Polypeptides Containing Multiple Functional Domains

The modular nature of the iMers of the disclosure is well-suited for designing complex functional domains that each contain multiple binding sites. iMers with functional domains comprising scFvs may include variants of scFvs such as tandem di-scFvs, tandem tri-scFvs, diabodies, tri(a)bodies, and tetrabodies. For example, a first functional domain in an iMer may comprise a di-scFv, which is produced by linking two VH and two VL regions. Thus, this single functional domain may be bispecific. If the second functional domain also comprises a di-scFv, the resulting iMer will exhibit quadrispecificity. In some aspects, the iMer comprises diabodies, or dimers of scFvs formed from pairs of scFvs whose linker peptides are otherwise too short to allow the two variable regions to fold together. Diabodies may be formed from a single contiguous peptide chain to form at least one functional domain of the present iMers.

Multiple Fab fragments may also be linked to form a functional domain that is bi- or tri-specific. For example, in a bi-specific Fab fragment, the variable and constant domains may be arranged in the following order from N-terminus to C-terminus: VL1, CL, first polypeptide linker, VH1, CH1, VL2, CH1, second polypeptide linker, VH2, CH1 (FIG. 27). The binding site created by VL1-VH1 may be different from the binding site created by VL2-VH2, creating a large functional domain that is bispecific. In certain aspects, a polypeptide linker may also be present between CH1 and VL2. When combined, via an interaction domain, with a second functional domain comprising a bispecific Fab fragment, the Double Fab Domain polypeptide may have specificity for up to four molecules. In a tri-specific Fab fragment, the variable and constant domains may be arranged from N-terminus to C-terminus as follows: VL1, CL, first polypeptide linker, VH1, CH1, VL2, CL, second polypeptide linker, VH2, CH1, VL3, CH1, third polypeptide linker, VH3, CH1 (FIG. 28). Three binding sites may be created by the combinations of VL1-VH1, VL2-VH2, and VL3-VH3. In certain aspects, polypeptide linkers may also be present between CH1 and VL2 or CH1 and VL3. When combined, via an interaction domain, with a second functional domain comprising a tri-specific Fab fragment, the Triple Fab Domain polypeptide may have specificity for up to six molecules.

In other aspects, a multiplicity of scFv and/or Fab fragments may be linked in this manner. Four, five, six, seven, eight or more scFv or Fab fragments may be linked to form a single functional domain. This multi-specific functional domain, if linked to an interaction domain, could be paired with a second interaction domain and another functional domain comprising multiple scFv or Fab fragments. In addition, linked scFv and/or Fab fragments with the same or differing numbers of binding sites could also be attached to the other ends of the interaction domains, for example, by adding additional functional domains. Thus, molecules of increasing complexity may be generated by linking combinations of scFv and/or Fab fragments. Formation of these complex multi-specific iMers may be facilitated by the use of linkers in between the antibody heavy and light chain regions, as described herein. Such molecules may have additional domains or linkers at the N-terminus, C-terminus or interspersed in the molecule.

4. Non-Immunoglobulin Interaction Domains

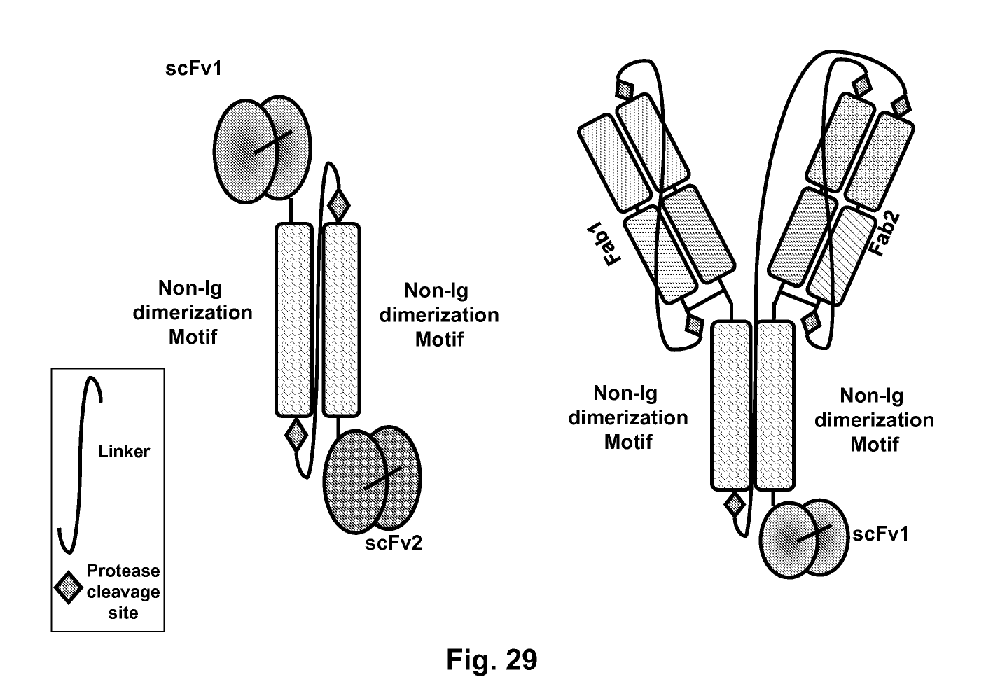

In some aspects, interaction domains (IDs) of iMers comprise non-immunoglobulin dimerization motifs. For example, an iMer may comprise functional domains (FDs) comprising antibody heavy and light chain regions and interaction domains (IDs) comprising two dimerization motifs which form a dimer (FIG. 29). An exemplary, non-limiting iMer comprises a first scFv, a first polypeptide linker, a first dimerization motif, a second polypeptide linker, a second dimerization motif, a third polypeptide linker, and a second scFv. The two scFvs confer multimeric binding. Another exemplary, non-limiting iMer comprises a polypeptide sequence comprising an antibody light chain comprising a variable domain (VL1) and a light constant domain 1 (LCD1), a first polypeptide linker, a polypeptide sequence comprising an antibody heavy chain comprising a variable (VH1) and a heavy constant domain 1 (HCD1), a first dimerization motif, a second polypeptide linker, a polypeptide sequence comprising an antibody light chain comprising a variable domain (VL2) and a light constant domain 2 (LCD2), a third polypeptide linker, a polypeptide sequence comprising an antibody heavy chain comprising a variable domain (VH2) and a heavy constant domain 2 (HCD2), a second dimerization motif, and a scFv. This exemplary, non-limiting iMer is multispecific. Other exemplary, non-limiting iMers comprise functional domains comprising non-immunoglobulin binding motifs linked to non-immunoglobulin interaction domains.

Interaction domains may be any monomers derived from proteins that dimerize or multimerize, primarily via non-covalent bonds and/or disulfide bonds, to form the quaternary structure of a protein. In some aspects, the interaction domain monomers may be identical, so that homodimers are formed. In other aspects, the interaction domain monomers may be different, resulting in formation of heterodimers. Dimerization motifs from specialized proteins may be used. Exemplary, non-limiting proteins containing dimerization motifs include but are not limited to receptor tyrosine kinases, transcription factors such as leucine zipper motif proteins and nuclear receptors, 14-3-3 proteins, G-protein coupled receptors, kinesin, triosephophateisomerase, alcohol dehydrogenase, Factor XI, Factor XIII, Toll-like receptor, fibrinogen, coil-coil homodimerization motifs such as Geminin, HIV major homology region, S. cerevisiae Sir4p, zinc-finger domains, viral coat proteins, and p53. By way of further example, the IDs of an iMer may comprise transmembrane domains from receptor tyrosine kinases (RTKs), which are single hydrophobic transmembrane domains comprising 25-38 amino acids.

The foregoing are non-limiting examples of non-Ig IDs. Dimerization motifs may, in certain aspects, be interconnected with other domains in the iMers through polypeptide linkers. Exemplary, non-limiting linkers interconnect the C terminus of a first dimerization motif to the N terminus of a second dimerization domain, or, alternately, interconnect the C-terminus of a first dimerization motif to a functional domain.

5. Non-Antibody Functional Domains

Functional domains (FDs) that are not portions of antibody molecules are also contemplated. By way of example, a portion of a polypeptide that specifically binds to another polypeptide may be used as a functional domain. Binding sites from a receptor-ligand pair are non-limiting examples of domains that, in a manner akin to an antibody-epitope interaction, may be used to target the iMer.

In certain aspects, a functional domain is a polypeptide portion comprising a ligand binding domain or a receptor binding domain. As used herein, a ligand binding domain is a portion of a receptor molecule that specifically binds to a site on a ligand. As used herein, a receptor binding domain is a portion of a ligand molecule that specifically binds to a site on a receptor. By way of example, tumor necrosis factor alpha is a ligand that binds to and signals via tumor necrosis factor alpha receptor. Thus, a polypeptide portion comprising a domain of TNFalpha that specifically binds to TNFalpha receptor could be used as a FD to target an iMer to cells expressing the TNFalpha receptor.

In additional aspects, functional domains may comprise ligands such as proteins, for example hormones, growth and/or survival factors, structural proteins, enzymes, cytokines, transport proteins, transmembrane proteins, nuclear proteins, proteins which bind other biomolecules, and/or binding domains derived from these proteins. Further exemplary functional domains include antibody mimetics, such as polypeptide scaffolds that mimic the structure of an antibody.

6. Combination Molecules

Multimeric polypeptides such as iMers may be formed from a single, contiguous polypeptide, or they may be assembled by combining more than one polypeptide. For example, a portion of an iMer may be produced as a single, contiguous polypeptide which is then combined with one or more additional polypeptides to form a complete iMer. The one or more additional polypeptides may also be single, contiguous polypeptides. As described above, certain iMers illustrated in FIG. 25 (iMer-3n, iMer-4-n, iMer-5n, and iMer-6n), are assembled from a single polypeptide comprising an antibody light chain comprising a variable domain (VL1) and a light constant domain 1 (LCD1), in combination with a second, more complex polypeptide.

For example, iMer-3n is formed by the assembly of an antibody light chain comprising a VL1 and an LCD1 and a single, contiguous polypeptide comprising, in the following orientation from N-terminus to C-terminus, (i) an antibody heavy chain comprising a VH1 and a HCD1, (ii) a polypeptide sequence comprising a polypeptide linker, and (iii) a polypeptide sequence comprising a HCD2.

Similarly, iMer-4-n is formed by the assembly of an antibody light chain comprising a VL1 and an LCD1 and a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising an scFv, (iv) a polypeptide sequence comprising a second polypeptide linker, and (v) a polypeptide sequence comprising a heavy constant domain 2 (HCD2).

The related iMer-5n is formed by the assembly of an antibody light chain comprising a VL1 and an LCD1 and a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising a first scFv, (iv) a polypeptide sequence comprising a second polypeptide linker, (v) a polypeptide sequence comprising a heavy constant domain 2 (HCD2), (vi) a polypeptide comprising a third polypeptide linker, and (vii) a polypeptide sequence comprising a second scFv.

Finally, iMer-6n is formed by the assembly of an antibody light chain comprising a VL1 and an LCD1 and a single, contiguous polypeptide comprising, in the following orientation from N-terminus to C-terminus, (i) a polypeptide sequence comprising an antibody heavy chain comprising a variable domain (VH1) and a heavy constant domain 1 (HCD1), (ii) a polypeptide sequence comprising a first polypeptide linker, (iii) a polypeptide sequence comprising an scFv, (iv) a polypeptide sequence comprising a second polypeptide linker, (v) a polypeptide sequence comprising a second scFv, (vi) a polypeptide sequence comprising a third polypeptide linker, (vii) a polypeptide sequence comprising an antibody heavy chain comprising a heavy constant domain 2 (HCD2), (viii) a polypeptide sequence comprising a fourth polypeptide linker, and (ix) a polypeptide sequence comprising a third scFv.

These iMers are intended for illustrative purposes only, and are not limiting. The present disclosure also contemplates iMers which have been formed, for example, from a single polypeptide comprising one or more antibody light chains and one or more antibody heavy chains, all linked by polypeptide linkers, wherein the single polypeptide forms an iMer by assembling with a single polypeptide of similar composition. In some aspects, a single polypeptide comprises, in the following orientation from N-terminus to C-terminus: (i) a polypeptide comprising an antibody light chain comprising a variable domain (VL1), a light constant domain 1 (LCD 1), (ii) a first polypeptide linker, (iii) a polypeptide comprising an antibody heavy chain comprising a variable domain (VH1) and a heavy constant domain 1 (HCD1), and further comprising an antibody light chain comprising a variable domain (VL2), a light constant domain 2 (LCD2), (iv) a polypeptide comprising a second polypeptide linker, and (v) a polypeptide comprising an antibody heavy chain comprising a variable domain (VH2) and a heavy constant domain (HCD2). This single polypeptide may be combined with a second polypeptide comprising a similar structure. In this manner, an iMab with a dual-Fab domain may be produced (an iMab-DFD, as in FIG. 27).

7. Labels, Conjugates and Moieties

iMers of the disclosure may be conjugated to labels for the purposes of diagnostics and other assays wherein the iMer and/or its associated ligand(s) may be detected. Labels include, without limitation, a chromophore, a fluorophore, a fluorescent protein, a phosphorescent dye, a tandem dye, a particle, a hapten, an enzyme and a radioisotope.

In certain aspects, the iMers are conjugated to a fluorophore. As such, fluorophores used to label iMers of the disclosure include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Pat. Nos. 6,977,305 and 6,974,873), a carbocyanine (including any corresponding compounds in U.S. Ser. No. 09/557,275; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343; 5,227,487; 5,442,045; 5,798,276; 5,846,737; 4,945,171; U.S. Ser. Nos. 09/129,015 and 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including any corresponding compounds disclosed in U.S. Pat. Nos. 5,696,157; 5,459,276; 5,501,980 and 5,830,912), a benzofuran (including any corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

In a specific aspect, the fluorophores conjugated to the iMers described herein include xanthene (rhodol, rhodamine, fluorescein and derivatives thereof) coumarin, cyanine, pyrene, oxazine and borapolyazaindacene. In other aspects, such fluorophores are sulfonated xanthenes, fluorinated xanthenes, sulfonated coumarins, fluorinated coumarins and sulfonated cyanines. Also included are dyes sold under the tradenames, and generally known as, Alexa Fluor, DyLight, Cy Dyes, BODIPY, Oregon Green, Pacific Blue, IRDyes, FAM, FITC, and ROX.

The choice of the fluorophore attached to the iMer will determine the absorption and fluorescence emission properties of the conjugated iMer. Physical properties of a fluorophore label that can be used for an iMer and iMer-bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. Other desirable properties of the fluorescent label may include cell permeability and low toxicity, for example if labeling of the iMer is to be performed in a cell or an organism (e.g., a living animal).

In certain aspects, an enzyme is a label and is conjugated to an iMer. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art.

In one aspect, a colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including AMPLEX® Red reagent and its variants (U.S. Pat. No. 4,384,042) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

In another aspect, a colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912), fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. In one aspect, fluorogenic substrates include resorufin, beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain aspects, fluorescent proteins may be conjugated to the iMers as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra.

In certain aspects, the label is a radioactive isotope. Examples of suitable radioactive materials include, but are not limited to, iodine (121I, 123I, 125I, 131I), carbon (14C), sulfur (35S), tritium (3H), indium (111In, 112In, 113mIn, 115mIn,), technetium (99Tc, 99mTc), thallium (201Ti), gallium (68Ga, 67Ga), palladium (103Pd), molybdenum (99Mo), xenon (135Xe), fluorine (18F), 153SM, 177Lu, 159Gd, 149Pm, 140La, 175Yb, 166Ho, 90Y, 47Sc, 186Re, 188Re, 142Pr, 105 Rh and 97 Ru.

In some aspects, drugs may be conjugated to the iMers. For example, an iMer comprising an scFv may be conjugated to a cytotoxic drug. In this example, the scFv may bind to a target antigen on a cell surface, and the cytotoxic drug is then delivered to the cell. In some aspects, the iMer conjugate is internalized, which releases the cytotoxic drug to the cell. Any cytotoxic drug known in the art may be conjugated to an iMer. Some antibody conjugates are already approved by the FDA or are currently undergoing clinical trials.

In certain features, drugs and other molecules may be targeted to iMers via site-specific conjugation. For example, iMers may comprise cysteine engineered domains (including cysteine(s) engineered into an FD and/or ID), which result in free thiol groups for conjugation reactions. Methods for generating stable cysteine engineered antibodies are described in U.S. Pat. No. 7,855,275, U.S. 20110033378 and WO 2011/005481, the contents of which are incorporated herein by reference in their entirety.

8. Exemplary Targets

In some aspects, specific pairs of molecules are targeted by iMers. iMers of the disclosure may be capable of binding pairs of cytokines selected from, for example, IL-1α and IL-1β; IL-12 and IL-18; TNFα and IL-23; TNFα and IL-13; TNF and IL-18; TNF and IL-12; TNF and IL-1beta; TNF and MIF; TNF and IL-17; and TNF and IL-15; TNF and VEGF; VEGFR and EGFR; IL-13 and IL-9; IL-13 and IL-4; IL-13 and IL-5; IL-13 and IL-25; IL-13 and TARC; IL-13 and MDC; IL-13 and MIF; IL-13 and TGF-β; IL-13 and LHR agonist; IL-13 and CL25; IL-13 and SPRR2a; IL-13 and SPRR2b; IL-13 and ADAMS; and TNFα and PGE4; IL-13 and PED2; TNF and PEG2; HER2 and HER3; HER1 and HER2; HER1 and HER3.

In certain aspects, iMers of the disclosure may be capable of binding pairs of targets selected from, for example, CD138 and CD20; CD138 and CD40; CD19 and CD20; CD20 and CD3; CD38 and CD138; CD38 and CD20; CD38 and CD40; CD40 and CD20; CD-8 and IL-6; CSPGs and RGM A; CTLA4 and BTNO2; IGF1 and IGF2; IGF1/2 and ErbB2; IGFR and EGFR; ErbB2 and ErbB3; ErbB2 and CD64; IL-12 and TWEAK; IL-13 and IL-113; MAG and RGM A; NgR and RGM A; NogoA and RGM A; OMGp and RGM A; PDL-1 and CTLA4; RGM A and RGM B; Te38 and TNFα; TNFα and Blys; TNFα and CD-22; TNFα and CTLA-4; TNFα and GP130; TNFα and IL-12p40; and TNFα and RANK ligand.

In some aspects, iMers of the disclosure may be capable of binding one, two or more growthfactors, cytokines, cytokine-related proteins, and receptors selected from among, for example, BMP1, BMP2, BMP3B (GDF10), BMP4, BMP6, BMP8, CSF1(M-CSF), CSF2 (GM-CSF), CSF3 (G-CSF), EPO, FGF1 (aFGF), FGF2 (bFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, FGFR, FGFR1, FGFR2, FGFR3, FGFR4, FGFRL1, FGFR6, IGF1, IGF2, IGF1R, IGF2R, IFNA1, IFNA2, IFNA4, IFNA5, IFNA6, IFNA7, IFNAR1, IFNAR2, IFNB1, IFNG, IFNW1, FIL1, FIL1 (EPSILON), FIL1 (ZETA), IL1A, IL1B, IL2, IL3, IL4, IL5, IL6, IL7, IL8, IL9, IL10, IL11, IL12A, IL12B, IL13, IL14, IL15, IL16, IL17, IL17B, IL18, IL19, IL20, IL22, IL23, IL24, IL25, IL26, IL27, IL28A, IL28B, IL29, IL30, IL2RA, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL11RA, IL12RB1, IL12RB2, IL13RA1, IL13RA2, IL15RA, IL17R, IL17RA, IL17RB, IL17RC, IL17RD, IL18R1, IL20RA, IL20RB, IL21R, IL22R, IL22RA1, IL23R, IL27RA, IL28RA, PDGFA, PDGFB, PDGFRA, PDGFRB, TGFA, TGFB1, TGFB2, TGFB3, TGFBR1, TGFBR2, TGFBR3, ACVRL1, GFRA1, LTA (TNF-b), LTB, TNF (TNF-a), TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, TNFRSF1A, TNFRSF1B, TNFRSF10A (Trail-receptor), TNFRSF10B (Trail-receptor 2), TNFRSF10C (Trail-receptor 3), TNFRSF10D (Trail-receptor 4), FIGF (VEGFD), VEGF, VEGFB, VEGFC, KDR, FLT1, FLT4, NRP1, IL1HY1, IL1RAP, IL1RAPL1, IL1RAPL2, IL1RN, IL6ST, IL18BP, IL18RAP, IL22RA2, AIF1, HGF, LEP (leptin), PTN, ALK and THPO.

In further aspects, iMers of the disclosure may be capable of binding one or more chemokines, chemokine receptors, and chemokine-related proteins selected from among, for example, CCL1(I-309), CCL2 (MCP-1/MCAF), CCL3 (MIP-1a), CCL4 (MIP-1b), CCL5 (RANTES), CCL7 (MCP-3), CCL8 (mcp-2), CCL11 (eotaxin), CCL13 (MCP-4), CCL15 (MIP-1d), CCL16 (HCC-4), CCL17 (TARC), CCL18 (PARC), CCL19 (MIP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCL1(GRO1), CXCL2 (GRO2), CXCL3 (GRO3), CXCL5 (ENA-78), CXCL6 (GCP-2), CXCL9 (MIG), CXCL10 (IP 10), CXCL11 (I-TAC), CXCL12 (SDF1), CXCL13, CXCL14, CXCL16, PF4 (CXCL4), PPBP (CXCL7), CX3CL1 (SCYD1), SCYE1, XCL1 (lymphotactin), XCL2 (SCM-1b), BLR1 (MDR15), CCBP2 (D6/JAB61), CCR1 (CKR1/HM145), CCR2 (mcp-1RB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 (CMKBR8/TER1/CKR-L1), CCR9 (GPR-9-6), CCRL1 (VSHK1), CCRL2 (L-CCR), XCR1 (GPRS/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8Rb), LTB4R (GPR16), TCP10, CKLFSF2, CKLFSF3, CKLFSF4, CKLFSF5, CKLFSF6, CKLFSF7, CKLFSF8, BDNF, C5R1, CSF3, GRCC10 (C10), EPO, FY (DARC), GDFS, HIF1A, IL8, PRL, RGS3, RGS13, SDF2, SLIT2, TLR2, TLR4, TREM1, TREM2, and VHL.

Other iMers may be capable of binding cell surface proteins selected from among, for example, integral membrane proteins including ion channels, ion pumps, G-protein coupled receptors, structural proteins, adhesion proteins such as integrins, transporters, membrane-bound enzymes, proteins involved in accumulation and transduction of energy and lipid-anchored proteins including G proteins and some membrane-anchored kinases. iMers may also be capable of binding enzymes such as kinases, proteases, lipases, phosphatases, fatty acid synthetases, digestive enzymes such as pepsin, trypsin, and chymotrypsin, lysozyme, and polymerases. iMers may also be capable of binding to receptors such as hormone receptors, lymphokine receptors, monokine receptors, growth factor receptors, G-protein coupled receptors, and more.

In some aspects, the multimeric nature of the iMers of the disclosure confers the ability to target labels or therapeutics to a specific cell type or molecular target. For example, one functional domain in an iMer may bind to a target at the surface of a cell, while another functional domain in the same iMer binds to a hapten or labeling agent useful for detection. Similarly, one functional domain may bind to a cellular target while a second functional domain binds to a toxin. Because both binding reactions are mediated through a single molecule, the toxin may be placed in the proximity of the cellular target, where it effects a cytotoxic function.

### B. Nucleic Acids Encoding Contiguous, Multimeric Polypeptides

The present disclosure provides nucleic acid molecules that encode iMers (such as iMabs). One aspect of the disclosure provides nucleic acid molecules encoding any of the iMers of the disclosure. A nucleic acid molecule may encode the iMer comprising at least two subunits, each of which includes at least a function domain (FD) and an interaction domain (ID), wherein the nucleic acid molecule comprises a nucleic acid portion comprising a nucleotide sequence that encodes a functional domain (FD1) that binds to a first binding site; a nucleic acid portion comprising a nucleotide sequence that encodes an interaction domain (ID1); a nucleic acid portion comprising a nucleotide sequence that encodes a functional domain (FD2) that binds to a second binding site; and a nucleic acid portion comprising a nucleotide sequence that encodes an interaction domain (ID2); wherein the nucleic acid molecule further comprises at least one nucleic acid portion comprising a nucleotide sequence encoding a polypeptide linker, wherein ID1 and ID2 are capable of associating with each other, and wherein the iMer is multispecific. The encoded polypeptide linker may have at least one cleavage site (e.g., protease cleavage site).

Like the polypeptides they encode, the nucleic acid molecules of the disclosure are contiguous nucleic acid molecules. Portions of the nucleic acid molecule may comprise one, two, three or more nucleotide sequences that encode polypeptide linkers. These portions may encode identical polypeptide linkers, or at least one of the polypeptide linkers may be different. In addition, the nucleic acid sequences encoding polypeptide linkers may include sequences encoding at least one cleavage site (e.g., a protease cleavage site), at least two cleavage sites, or at least three cleavage sites. Moreover, molecules may also include one or more additional linkers that do not include any cleavage sites. For example, when a nucleic acid molecule encodes an iMer that includes an scFv, the components of the scFv may be interconnect by a linker that may not be a cleavage linker.

In certain aspects, segments of the nucleic acid molecule comprise nucleic acid sequences encoding interaction domains that are dimerization motifs from specialized proteins, as described above. In addition, nucleic acids may encode functional domains that are receptor binding domains, ligand binding domains, or functional domains that include an antigen binding portion of an antibody.

In some aspects, segments of the nucleic acid molecule comprise nucleic acid sequences encoding functional domains comprising immunoglobulin domains. For example, the nucleic acid molecule may encode one or more antigen binding portions of an antibody. The nucleic acid segment may encode an antibody light chain and an antibody heavy chain. For example, the antibody light chain may comprise a variable domain (VL1) and a light constant domain 1 (LCD 1), and the antibody heavy chain may comprise a variable domain (VH1) and a heavy constant domain 1 (HCD1). The same nucleic acid molecule may also have segments that encode a second antibody light chain comprising a variable domain (VL2) and a light constant domain 2 (LCD2), as well as a second antibody heavy chain comprising a variable domain (VH2) and a heavy constant domain 2 (HCD2).

In other aspects, the domains of the iMer may be arranged in specific orientation. For example, the nucleic acid may comprise nucleic acid segments in the following orientation from 5′ to 3′: (i) the nucleic acid segment comprising the nucleotide sequence that encodes an antibody light chain comprising the variable domain (VL1) and the light constant domain 1 (LCD1); (ii) the nucleic acid segment comprising the nucleotide sequence that encodes an antibody heavy chain comprising the variable domain (VH1) and the heavy constant domain 1 (HCD1); (iii) the nucleic acid segment comprising the nucleotide sequence that encodes an antibody light chain comprising the variable domain (VL2) and the light constant domain 2 (LCD2); and (iv) the nucleic acid segment comprising the nucleotide sequence that encodes an antibody heavy chain comprising the variable domain (VH2) and the heavy constant domain 2 (HCD2). Note that any one or more of these segments may, in certain features, be interconnected via a nucleic acid segment comprising a nucleotide sequence that encodes a polypeptide linker.

Genes encoding domains of the heavy and light chain antibodies are found at the immunoglobulin heavy chain locus (IGH), immunoglobulin kappa locus (IGκ), and immunoglobulin lambda locus (IGλ). When recombined, the genes give rise to variable regions with an extensive array of binding specificities. Genes may be selected on the basis of the binding properties of the variable regions, or, alternately, genes may be engineered to substitute specific residues into the variable regions. In some aspects, the nucleic sequences encoding antibody light and/or heavy chain variable regions represented by VL1, VH1, VL2, and VH2 are different, giving rise to a polypeptide with bispecificity (or even further multispecificty). In addition, the LCD1 may or may not be the same as the LCD2. HCD1 and HCD2 may comprise one or more of a CHL CH2, and CH3 region. As with the LCDs, the HCD1 and HCD2 may be the same, or may be different.

A portion of the nucleic acid molecule that encodes a heavy constant domain (HCD) may comprise nucleotide sequences that encode a hinge region. In particular, the encoded heavy constant domain 1 (HCD1) and/or the heavy constant domain 2 (HCD2) may comprise a hinge region.

In some aspects, nucleic acid molecules encoding iMers of the disclosure may comprise nucleotide sequences which encode polypeptide linkers, wherein the polypeptide linkers join other domains and/or regions of the iMer. The nucleic acid molecule may comprise one, two, three, or more than three nucleic acid segments each of which comprise a nucleotide sequence that encodes a polypeptide linker. If a nucleic acid molecule comprises a plurality of nucleotide sequences that each encode a polypeptide linker, each of the nucleotide sequences may encode distinct polypeptide linkers, or two or more of the sequences may encode the same polypeptide linkers. Moreover, one or more of the polypeptide linkers may be cleavable linkers, and the molecule may include a combination of cleavable linkers and one or more non-cleavable linkers.

For the nucleic acid molecule described above, comprising nucleic acid segments (i)-(iv), nucleic acid segments encoding polypeptide linkers may, for example, be located between segment (i) and segment (ii), segment (ii) and segment (iii), and/or segment (iii) and segment (iv). A non-limiting exemplary nucleic acid molecule comprises: a nucleic acid segment comprising a nucleotide sequence that encodes an antibody light chain comprising a variable domain (VL1) and a light constant domain 1 (LCD1); a nucleic acid segment comprising a nucleotide sequence that encodes a first polypeptide linker; a nucleic acid segment comprising a nucleotide sequence that encodes an antibody heavy chain comprising a variable domain (VH1) and a heavy constant domain 1 (HCD1); a nucleic acid segment comprising a nucleotide sequence that encodes a second polypeptide linker; a nucleic acid segment comprising a nucleotide sequence that encodes an antibody light chain comprising a variable domain (VL2) and a light constant domain 2 (LCD2); a nucleic acid segment comprising a nucleotide sequence that encodes a third polypeptide linker; and a nucleic acid segment comprising a nucleotide sequence that encodes an antibody heavy chain comprising a variable domain (VH2) and a heavy constant domain 2 (HCD2); wherein the contiguous polypeptide is a multispecific polypeptide. The foregoing is merely exemplary, and the iMer may include additional linkers, and/or a combination of cleavable and non-cleavable linkers.

iMers encoded by the nucleic acid molecules described in the present disclosure are translated as a single chain and assembled with all polypeptide linkers, if present, in place. However, all or a subset of such linker may be removed after secondary and tertiary structures have formed between the domains of the polypeptide. Accordingly, all or a subset of the polypeptide linkers, if present, may contain one or more cleavage sites. Nucleic acid segments comprising nucleotide sequences encoding polypeptide linkers similarly may comprise nucleotide sequences encoding one or more cleavage sites (e.g., protease cleavage sites). For example, a nucleic acid molecule may comprise at least two nucleic acid segments each of which comprises a nucleotide sequence that encodes a polypeptide linker with cleavage sites. In some aspects, at least one of the nucleotide sequences encodes a polypeptide linker comprising at least one cleavage site (e.g., a protease cleavage site). The nucleotide sequence may encode a polypeptide linker comprising two cleavage sites. For a nucleic acid molecule comprising three nucleic acid segments each of which comprises a nucleotide sequence that encodes a polypeptide linker, at least one of the polypeptide linkers may comprise a cleavage site. Alternately, two or all three of the polypeptide linkers may comprise at least one cleavage site, for example two cleavage sites. Polypeptide linkers and cleavage sites in an iMer may be different from one another, and similarly, the nucleotide sequences encoding the polypeptide linkers and/or the cleavage sites may be different. Moreover, a nucleic acid molecule may also encode one or more polypeptide linkers that are not cleavable, and thus do not contain a cleavage site. In certain aspects, a nucleic acid molecule encodes one or more polypeptide linkers comprising at least one protease cleavage site.

Additional aspects of the nucleic acid molecules correspond to the various multimeric polypeptides (such as iMers and iMabs) described above. For example, the nucleic acid sequence encoding an iMer (such as an iMab) may comprise a nucleic acid segment comprising a nucleotide sequence that encodes an antibody light chain comprising a variable domain (VL1) and a light constant domain 1 (LCD 1), a nucleic acid segment comprising a nucleotide sequence that encodes a first polypeptide linker, a nucleic acid segment comprising a nucleotide sequence that encodes an antibody heavy chain comprising a variable domain (VH1) and a heavy constant domain 1 (HCD1), a nucleic acid segment comprising a nucleotide sequence that encodes a second polypeptide linker, a nucleic acid segment comprising a nucleotide sequence that encodes an antibody light chain comprising a variable domain (VL2) and a light constant domain 2 (LCD2), a nucleic acid segment comprising a nucleotide sequence that encodes a third polypeptide linker, and a nucleic acid segment comprising a nucleotide sequence that encodes an antibody heavy chain comprising a variable domain (VH2) and a heavy constant domain 2 (HCD2), wherein the nucleic acid molecule encodes an iMer (such as an iMab). In certain features, the nucleic acid molecules encoding an iMer may comprise nucleic acid segments arranged in the following orientation from N-terminus to C-terminus: (i) a nucleic acid segment comprising a nucleotide sequence that encodes an antibody light chain comprising VL1 and LCD1, (ii) a nucleic acid segment comprising a nucleotide sequence that encodes a first polypeptide linker, (iii) a nucleic acid segment comprising a nucleotide sequence that encodes an antibody heavy chain comprising VH1 and HCD1, (iv) a nucleic acid segment comprising a nucleotide sequence that encodes a second polypeptide linker, (v) a nucleic acid segment comprising a nucleotide sequence that encodes an antibody light chain comprising VL2 and LCD2, (vi) a nucleic acid segment comprising a nucleotide sequence that encodes a third polypeptide linker, and (vii) a nucleic acid segment comprising a nucleotide sequence that encodes an antibody heavy chain comprising VH2 and HCD2. A non-limiting example of the iMer encoded by this nucleic acid molecule is illustrated in FIG. 8. In certain aspects all of the polypeptide linkers comprise at least one protease cleavage site. Such molecules may have additional domains or linkers at the N-terminus, C-terminus, or interspersed in the molecule.

By way of another example, the nucleic acid molecule encoding another iMer may comprise a nucleic acid segment comprising a nucleotide sequence encoding an antibody light chain comprising a variable domain (VL1) and a light constant domain 1 (LCD1), a nucleic acid segment comprising a nucleotide sequence encoding a first polypeptide linker, a nucleic acid segment comprising a nucleotide sequence encoding an antibody heavy chain comprising a variable domain (VH1) and a heavy constant domain 1 (HCD1), a nucleic acid segment comprising a nucleotide sequence encoding a second polypeptide linker, a nucleic acid segment comprising a nucleotide sequence encoding a heavy constant domain 2 (HCD2), wherein the nucleic acid sequence encodes an iMer that binds one or more epitopes. In some aspects, the nucleic acid sequence encoding this iMer comprises nucleic acid segments arranged in the following orientation from N-terminus to C-terminus: (i) a nucleic acid segment comprising a nucleotide sequence encoding an antibody light chain comprising VL1 and LCD1, (ii) a nucleic acid segment comprising a nucleotide sequence encoding a first polypeptide linker, (iii) a nucleic acid segment comprising a nucleotide sequence encoding an antibody heavy chain comprising VH1 and HCD1, (iv) a nucleic acid segment comprising a nucleotide sequence encoding a second polypeptide linker, and (v) a nucleic acid segment comprising a nucleotide sequence encoding a HCD2. A non-limiting example of the iMer encoded by this nucleic acid molecule is illustrated as iMer-3 in FIG. 25. In certain aspects, all of the polypeptide linkers comprise at least one protease cleavage site. Such molecules may have additional domains or linkers at the N-terminus, C-terminus, or interspersed in the molecule.

In another aspect, a nucleic acid molecule comprising a nucleic acid segment encoding an antibody light chain comprising a VL1 and an LCD1 is expressed separately from a nucleic acid molecule comprising nucleic acid segments arranged in the following orientation from N-terminus to C-terminus, (i) a nucleic acid segment comprising a nucleotide sequence encoding an antibody heavy chain comprising a VH1 and a HCD1, (ii) a nucleic acid segment comprising a nucleotide sequence encoding a polypeptide linker, and (iii) a nucleic acid segment comprising a nucleotide sequence encoding a HCD2. Following expression of the two non-contiguous nucleic acid molecules, the polypeptide sequences can assemble into a single iMer. A non-limiting example of an iMer encoded by these nucleic acid molecules is illustrated as iMer-3n in FIG. 25. Such molecules may have additional domains or linkers at the N-terminus, C-terminus, or interspersed in the molecule. This is an example of an iMer in which a portion of the iMer is encoded by a contiguous nucleic acid molecule that encodes a contiguous polypeptide chain, but the entire iMer further includes one or more additional portions. In certain aspects, the polypeptide linker comprises at least one protease cleavage site. Such molecules may have additional domains or linkers at the N-terminus, C-terminus, or interspersed in the molecule.

The nucleic acid sequence encoding still another iMer comprises nucleic acid segments arranged in the following orientation from N-terminus to C-terminus: (i) a nucleic acid segment comprising a nucleotide sequence encoding an scFv, (ii) a nucleic acid segment comprising a nucleotide sequence encoding a first polypeptide linker, (iii) a nucleic acid segment comprising a nucleotide sequence encoding a heavy constant domain 1 (HCD1), (iv) a nucleic acid segment comprising a nucleotide sequence encoding a second polypeptide linker, (v) a nucleic acid segment comprising a nucleotide sequence encoding a second scFv, (vi) a nucleic acid segment comprising a nucleotide sequence encoding a third polypeptide linker, (vii) a nucleic acid segment comprising a nucleotide sequence encoding a third scFv, (viii) a nucleic acid segment comprising a nucleotide sequence encoding a fourth polypeptide linker, (ix) a nucleic acid segment comprising a nucleotide sequence encoding a heavy constant domain 2 (HCD2), (x) a nucleic acid segment comprising a nucleotide sequence encoding a fifth polypeptide linker, and (xi) a nucleic acid segment comprising a nucleotide sequence encoding a fourth scFv. The HCD1 and HCD2 may comprise, for example, CH2 and CH3 domains. A non-limiting example of the iMer encoded by such a nucleic acid molecules is illustrated as iMer-2 in FIG. 25. In certain aspects, the third polypeptide linker comprises at least one protease cleavage site. Such molecules may have additional domains or linkers at the N-terminus, C-terminus, or interspersed in the molecule.

The nucleic acid sequence encoding another iMer comprises nucleic acid segments arranged in the following orientation from N-terminus to C-terminus: (i) a nucleic acid segment comprising a nucleotide sequence encoding an antibody light chain comprising a variable domain (VL1) and a light constant domain (LCD1), (ii) a nucleic acid segment comprising a nucleotide sequence encoding a first polypeptide linker, (iii) a nucleic acid segment comprising a nucleotide sequence encoding an antibody heavy chain comprising a variable domain (VH1) and a heavy constant domain 1 (HCD1), (iv) a nucleic acid segment comprising a nucleotide sequence encoding a second polypeptide linker, (v) a nucleic acid segment comprising a nucleotide sequence encoding comprising an scFv, (vi) a nucleic acid segment comprising a nucleotide sequence encoding a third polypeptide linker, and (vii) a nucleic acid segment comprising a nucleotide sequence encoding a heavy constant domain 2 (HCD2). A non-limiting example of the Mab encoded by this nucleic acid sequence is illustrated as iMer-4 in FIG. 25. In certain aspects, the first and third polypeptide linkers each comprise at least one protease cleavage site. Such molecules may have additional domains or linkers at the N-terminus, C-terminus, or interspersed in the molecule.

In a similar aspect, a nucleic acid sequence comprising a nucleic acid segment encoding an antibody light chain comprising a VL1 and an LCD1 may be expressed separately from a nucleic acid sequence comprising nucleic acid segments arranged in the following orientation from N-terminus to C-terminus, (i) a nucleic acid segment comprising a nucleotide encoding an antibody heavy chain comprising a variable domain (VH1) and a heavy constant domain 1 (HCD1), (ii) a nucleic acid segment comprising a nucleotide encoding a first polypeptide linker, (iii) a nucleic acid segment comprising a nucleotide encoding an scFv, (iv) a nucleic acid segment comprising a nucleotide encoding a second polypeptide linker, and (v) a nucleic acid segment comprising a nucleotide encoding an a heavy constant domain 2 (HCD2). Following expression of the two nucleic acid sequences, the polypeptide sequences are assembled into a single iMab. A non-limiting example of an iMab encoded by these nucleic acids is illustrated as iMer-4-n in FIG. 25. This is another example of an iMer in which a portion of the iMer molecule is made as a contiguous polypeptide chain, but the entire iMer further includes one or more additional portions. In certain aspects, the second polypeptide linker comprises at least one protease cleavage site. Such molecules may have additional domains or linkers at the N-terminus, C-terminus, or interspersed in the molecule.

The nucleic acid sequence encoding another iMer comprises nucleic acid segments arranged in the following orientation from N-terminus to C-terminus: (i) a nucleic acid segment comprising a nucleotide encoding an antibody light chain comprising a variable domain (VL1) and a light constant domain (LCD1), (ii) a nucleic acid segment comprising a nucleotide encoding a first polypeptide linker, (iii) a nucleic acid segment comprising a nucleotide encoding an antibody heavy chain comprising a variable domain (VH1) and a heavy constant domain 1 (HCD1), (iv) a nucleic acid segment comprising a nucleotide encoding a second polypeptide linker, (v) a nucleic acid segment comprising a nucleotide encoding a first scFv, (vi) a nucleic acid segment comprising a nucleotide encoding a third polypeptide linker, (vii) a nucleic acid segment comprising a nucleotide encoding a heavy constant domain 2 (HCD2), (viii) a nucleic acid segment comprising a nucleotide encoding a fourth polypeptide linker, and (ix) a nucleic acid segment comprising a nucleotide encoding a second scFv. A non-limiting example of an iMer encoded by this nucleic acid sequence is illustrated as iMer-5 in FIG. 25. In certain aspects, the first and third polypeptide linker each comprise at least one protease cleavage site. Such molecules may have additional domains or linkers at the N-terminus, C-terminus, or interspersed in the molecule.

In a related aspect, a nucleic acid sequence comprising a nucleic acid segment encoding an antibody light chain comprising a VL1 and an LCD1 is expressed separately from a nucleic acid sequence comprising nucleic acid segments arranged in the following orientation from N-terminus to C-terminus: (i) a nucleic acid segment comprising a nucleotide encoding an antibody heavy chain comprising a variable domain (VH1) and a heavy constant domain 1 (HCD1), (ii) a nucleic acid segment comprising a nucleotide encoding a first polypeptide linker, (iii) a nucleic acid segment comprising a nucleotide encoding a first scFv, (iv) a nucleic acid segment comprising a nucleotide encoding a second polypeptide linker, (v) a nucleic acid segment comprising a nucleotide encoding a heavy constant domain 2 (HCD2), (vi) a nucleic acid segment comprising a nucleotide encoding a third polypeptide linker, and (vii) a nucleic acid segment comprising a nucleotide encoding a second scFv. Following expression of the two nucleic acid sequences, the polypeptide sequences are assembled into a single iMer. A non-limiting example of an iMer encoded by these nucleic acids is illustrated as iMer-5n in FIG. 25. This is yet another example of an iMer in which a portion of the iMer molecule is made as a contiguous polypeptide chain, but the entire molecule includes one or more additional portions. In certain aspects, the second polypeptide linker comprises at least one protease cleavage site. Such molecules may have additional domains or linkers at the N-terminus, C-terminus, or interspersed in the molecule.

The nucleic acid molecule encoding another iMer comprises nucleic acid segments arranged in the following orientation from N-terminus to C-terminus: (i) a nucleic acid segment comprising a nucleotide encoding an antibody light chain comprising a variable domain (VL1) and a light constant domain (LCD1), (ii) a nucleic acid segment comprising a nucleotide encoding a first polypeptide linker, (iii) a nucleic acid segment comprising a nucleotide encoding an antibody heavy chain comprising a variable domain (VH1) and a heavy constant domain 1 (HCD1), (iv) a nucleic acid segment comprising a nucleotide encoding a second polypeptide linker, (v) a nucleic acid segment comprising a nucleotide encoding a first scFv, (vi) a nucleic acid segment comprising a nucleotide encoding a third polypeptide linker, (vii) a nucleic acid segment comprising a nucleotide encoding a second scFv, (viii) a nucleic acid segment comprising a nucleotide encoding a fourth polypeptide linker, (ix) a nucleic acid segment comprising a nucleotide encoding a heavy constant domain 2 (HCD2), (x) a nucleic acid segment comprising a nucleotide encoding a fifth polypeptide linker, and (xi) a nucleic acid segment comprising a nucleotide encoding a third scFv. A non-limiting example of an iMer encoded by this nucleic acid is illustrated as iMer-6 in FIG. 25. In certain aspects, the first and third polypeptide linker each comprise at least one protease cleavage site. Such molecules may have additional domains or linkers at the N-terminus, C-terminus, or interspersed in the molecule.

In another aspect, a nucleic acid molecule comprising a nucleic acid segment encoding an antibody light chain comprising a VL1 and an LCD1 is expressed separately from a nucleic acid sequence comprising nucleic acid segments arranged in the following orientation from N-terminus to C-terminus: (i) a nucleic acid segment comprising a nucleotide encoding an antibody heavy chain comprising a variable domain (VH1) and a heavy constant domain 1 (HCD1), (ii) a nucleic acid segment comprising a nucleotide encoding a first polypeptide linker, (iii) a nucleic acid segment comprising a nucleotide encoding an scFv, (iv) a nucleic acid segment comprising a nucleotide encoding a second polypeptide linker, (v) a nucleic acid segment comprising a nucleotide encoding a second scFv, (vi) a nucleic acid segment comprising a nucleotide encoding a third polypeptide linker, (vii) a nucleic acid segment comprising a nucleotide encoding a heavy constant domain 2 (HCD2), (viii) a nucleic acid segment comprising a nucleotide encoding a fourth polypeptide linker, and (ix) a nucleic acid segment comprising a nucleotide encoding a third scFv. Following expression of the two nucleic acid sequences, the polypeptide sequences are assembled into a single iMab. A non-limiting example of an iMer encoded by these nucleic acids is illustrated as iMer-6n in FIG. 25. This is yet another example of an iMer in which a portion of the iMer molecule is made as a contiguous polypeptide chain, but the entire molecule includes one or more additional portions. In certain aspects, the second polypeptide linker comprises at least one protease cleavage site. Such molecules may have additional domains or linkers at the N-terminus, C-terminus, or interspersed in the molecule.

The nucleic acid sequence encoding a further iMer comprises nucleic acid segments arranged in the following orientation from N-terminus to C-terminus: (i) a nucleic acid segment comprising a nucleotide encoding a first scFv, (ii) a nucleic acid segment comprising a nucleotide encoding a first polypeptide linker, (iii) a nucleic acid segment comprising a nucleotide encoding a heavy constant domain 1 (HCD1), (iv) a nucleic acid segment comprising a nucleotide encoding a second polypeptide linker, (v) a nucleic acid segment comprising a nucleotide encoding a second scFv, (vi) a nucleic acid segment comprising a nucleotide encoding a third polypeptide linker, and (vii) a nucleic acid segment comprising a nucleotide encoding a heavy chain constant domain 2 (HCD2). A non-limiting example of the Mab encoded by this nucleic acid is illustrated as iMer-7 in FIG. 25. In certain aspects, the second polypeptide linker comprises at least one protease cleavage site. Such molecules may have additional domains or linkers at the N-terminus, C-terminus, or interspersed in the molecule.

The nucleic acid sequence encoding still another iMer comprises nucleic acid segments arranged in the following orientation from N-terminus to C-terminus: (i) a nucleic acid segment comprising a nucleotide encoding an antibody heavy chain comprising a variable domain (VH1) and a heavy constant domain 1 (HCD1), (ii) a nucleic acid segment comprising a nucleotide encoding a first polypeptide linker, (iii) a nucleic acid segment comprising a nucleotide encoding an antibody light chain comprising a variable domain (VL2) and a light constant domain 2 (LCD2), (iv) a nucleic acid segment comprising a nucleotide encoding a second polypeptide linker, and (iv) a nucleic acid segment comprising a nucleotide encoding an antibody heavy chain comprising a variable domain (VH2) and a heavy constant domain 2 (HCD2). This nucleic acid sequence is expressed separately from a nucleic acid sequence comprising a nucleic acid segment encoding an antibody light chain comprising a variable domain (VL1) and a light chain constant domain (LCD1). Following expression of the two nucleic acids, the two polypeptides assemble into a single iMab. A non-limiting example of an iMer encoded by these two nucleic acids is illustrated as iMer-8 in FIG. 25. In certain aspects, the polypeptide linkers comprises at least one protease cleavage site. This is yet another example of an iMer in which a portion of the iMer molecule is made as a contiguous polypeptide chain, but the entire molecule includes one or more additional portions. Such molecules may have additional domains or linkers at the N-terminus, C-terminus, or interspersed in the molecule.

These nucleic acid molecules encoding iMers (such as iMabs) are intended for illustrative purposes only, and are not limiting. The present disclosure also contemplates nucleic acids encoding iMers which are formed after expression of a nucleic acid encoding a single polypeptide comprising one or more antibody light chains and one or more antibody heavy chains, all of which chains linked by polypeptide linkers. Thus, a nucleic acid sequence may encode a single polypeptide which forms an iMer after assembling with a single polypeptide of similar composition, also encoded by a single nucleic acid sequence. The single polypeptide encoded by the single nucleic acid may be combined with a second polypeptide comprising a similar structure, which was also encoded by the same or a different single nucleic acid. Thus, a single nucleic acid molecule may encode single polypeptides that, together, make up an iMer. Non limiting examples of such an iMer comprise a dual-Fab domain (an iMab-DFD, as in FIG. 27) or a triple-Fab domain (an iMab-TFD, as in FIG. 28).

As described herein, single nucleic acid sequences may encode single polypeptides that are assembled to produce iMers. A portion of an iMer may be encoded by a single nucleic acid sequence, which is then expressed as a single polypeptide that is, in turn, combined with one or more additional polypeptides (also encoded by single nucleic acid sequences) to form a complete iMer.

Another aspect of the disclosure provides a vector comprising a nucleic acid molecule or molecules as described herein, wherein the vector encodes an iMer (such as an iMab) as described herein.

A further aspect provides a host cell transformed with any of the nucleic acid molecules as described herein. In another aspect of the disclosure there is provided a host cell comprising the vector comprising nucleic acid molecules as described herein. In one aspect the host cell may comprise more than one vector.

### C. Methods for Producing Contiguous Multimeric Polypeptides

The present disclosure provides methods for producing iMers, such as iMabs. In certain aspects, the recombinant nucleic acids may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In certain aspects, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary, non-limiting regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This disclosure also pertains to a host cell transfected with a recombinant gene which encodes an iMer of the disclosure. The host cell may be any prokaryotic or eukaryotic cell. For example, an iMer may be expressed in bacterial cells such as E. coli, insect cells (e.g., using a baculovirus expression system), yeast, or mammalian cells. Other suitable host cells are known to those skilled in the art.

The present disclosure further pertains to methods of producing an iMer of the disclosure. For example, a host cell transfected with an expression vector encoding an iMer can be cultured under appropriate conditions to allow expression of the polypeptide to occur. The iMer may be secreted and isolated from a mixture of cells and medium containing the polypeptide. Alternatively, the iMer may be retained in the cytoplasm or in a membrane fraction and the cells harvested, lysed and the protein isolated. A cell culture includes host cells, media and other byproducts. Suitable media for cell culture are well known in the art. iMers can be isolated from cell culture medium, host cells, or both using techniques known in the art for purifying proteins, including ion-exchange chromatography, gel filtration chromatography, ultrafiltration, electrophoresis, and immunoaffinity purification. In certain aspects, the iMer is made as a fusion protein containing a domain which facilitates its purification.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. In certain aspects, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another aspect, the fusion gene can be synthesized by conventional techniques including automated DNA synthesizers. Alternatively, PCR amplification of gene fragments can be carried out using anchor primers which give rise to complementary overhangs between two consecutive nucleic acid fragments which can subsequently be annealed to generate a chimeric gene sequence (see, for example, Current Protocols in Molecular Biology, eds. Ausubel et al., John Wiley & Sons: 1992).

In some aspects, an expression vector expressing any of the nucleic acids described above may be used to express multimeric polypeptides such as iMers or iMabs in a host cell. Exemplary, non-limiting host cells include, but are not limited to, HEK293F and CHO cells. iMers (such as iMabs) may be isolated and purified according to methods known in the art. For example, the culture media from a culture of transfected cells may be applied to one or more columns in order to isolate and affinity purify the iMers of the disclosure. If desired, the molecular weight of the iMer can be confirmed, for example, by size-exclusion chromatography.

Once a molecule has been produced, it may be purified by any method known in the art for purification of an immunoglobulin molecule or other multimeric molecules, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the molecules of the present disclosure or fragments thereof may be fused to heterologous polypeptide sequences (referred to herein as “tags”) described above or otherwise known in the art to facilitate purification.

When using recombinant techniques, the molecule can be produced intracellularly, in the periplasmic space, or directly secreted into the medium. If the molecule is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies which are secreted into the periplasmic space of E. coli. Where the molecule is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain, if present, in the molecule and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the molecule to be recovered.

Following any preliminary purification step(s), the mixture comprising the molecule of interest and contaminants may be subjected to low pH hydrophobic interaction chromatography using an elution buffer at a pH between about 2.5-4.5, and performed at low salt concentrations (e.g., from about 0-0.25 M salt).

An iMer may be made and purified using, for example, any one or combination of techniques set forth in the Examples. Solely to illustrate, an iMer may be affinity purified using standard protein A affinity chromatography using HiTrap rProteinA FF column that has been equilibrated in PBS 1×. The iMer may be loaded on the column, which may be washed to eliminate contaminants and unbound material. Washing may be done with PBS 1× until the A280 trace reaches baseline. The bound protein may then be eluted in 25 mM glycine pH 2.8. Fractions may be immediately neutralized by addition of 0.1 volumes 1M Tris-HCl buffer pH 8. Fractions may then be analyzed for their iMer content by reading their absorbance at A280. The fractions containing the iMer may be pooled together and dialyzed overnight using a dialysis membrane cutoff of 10,000 kiloDalton (kDa), at 4° C. in 10× volume of PBS 1×. The dialyzed iMer may then be filtered using 0.22 micron filters and analyzed by reducing and non-reducing SDS-PAGE, for example a 4-12% Nupage gel run in MOPS buffer.

Following protein A purification, the iMer may be analyzed by analytical size-exclusion chromatography in order to determine the molecular weights (MW, Dalton) and the monomeric content of constructs. SEC-HPLC may be carried out using a TSK-GEL G3000SWXL column (Tosoh Bioscience LLC, Montgomeryville, Pa.), which separates globular proteins with MW that range from approximately 10 to 500 kDa, with a buffer containing 100 mM sodium phosphate, pH 6.8, and at a flow rate of 1 ml/min.

Regardless of how an iMer is purified, to confirm functional binding of the iMers of the disclosure (which includes iMabs), binding assays may be performed (before and/or after purification). For example, for bispecific or multispecific polypeptides comprising immunoglobulin domains, dual ELISA assays may be used. In some aspects, a first antigen is coated on a well, and binding to this antigen immobilizes the bispecific or multispecific polypeptide. A tagged second antigen is added to the well, and detected. Only bispecific molecules that are both immobilized via binding to the first antigen and also bound to the second antigen will be detected.

Verification of a homogeneous preparation of an iMer may be confirmed by an ELISA depletion assay, in which an iMer is pre-incubated on a first antigen, and the well supernatant is probed for the second antigen. If the well supernatant of the pre-absorbed signal shows a minimal signal, as compared to uni-specific controls which are specific only for the second antigen, the iMer is believed to be homogeneous.

Following production, polypeptide linkers that interconnect portions of the polypeptide may be cleaved. As detailed in the examples, such cleavage may occur at any point following production of the protein. For example, cleavage of linkers comprising protease cleavage sites may occur in the context of the cell culture system by adding protease to the cell culture media. Alternatively, cleavage may occur subsequent to affinity chromatography or other methodology used to purify the desired polypeptide species away from media components, cellular components and the like.

Cleavage of the polypeptide linkers may be mediated by treatment with a protease, for example, thrombin or furin. The polypeptide linkers may be engineered to include a protease cleavage site specific for virtually any protease, such that contacting the polypeptide with that protease specifically cleaves the polypeptide linkers. When multiple cleavage sites are present, the concentration of protease and/or the period of time during which the polypeptide is treated with the protease may be manipulated to influence whether cleavage occurs at every site or at fewer that all of the sites (partial cleavage).

Removal of the linkers may be mediated by incubation of the iMer with the pertinent protease (such as thrombin, as used in the Examples). The protease digestion may be analyzed using SDS-PAGE analysis. Intact iMers, without protease treatment and under denaturing and reducing conditions, run as a distinct single band, whereas treatment with the protease releases the individual components of the iMers. In addition to the reducing SDS-PAGE analysis, linker removal may also be analyzed by SEC-HPLC. Prior to protease treatment, an intact iMer may have two major conformational peaks; however, after protease treatment an iMer may have a near 100% monomeric and homogeneous peak.

Regardless of whether cleavage occurs at each of the engineered cleavage sites or at only a subset of the engineered cleavage sites (partial), the disclosure contemplates that small portions of the linker sequence may remain following cleavage (e.g., 5, 4, 3, 2, 1 residue attached to any one or more portions of the functional molecule). Moreover, it is recognized that the iMer may include a combination of linkers, some of which are cleavable and some of which are not.

### D. Pharmaceutical Formulations

In certain aspects, the disclosure provides pharmaceutical compositions. Such pharmaceutical compositions may be compositions comprising a nucleic acid molecule that encodes an iMer (which, in certain features, may be an iMab). Such pharmaceutical compositions may also be compositions comprising an iMer, or a combination of iMers, and a pharmaceutically acceptable excipient. Note that, if the iMer has already been subjected to protease digestion to remove one or more linkers, the composition or pharmaceutical composition may include an iMer that, prior to exposure to protease, was a contiguous polypeptide chain under reducing conditions. In certain aspects, the pharmaceutical compositions of the disclosure are used as a medicament.

In certain aspects, iMers or a combination of iMers (or nucleic acid molecules or contiguous, multimeric polypeptides) may be formulated with a pharmaceutically acceptable carrier, excipient or stabilizer, as pharmaceutical compositions. In certain aspects, such pharmaceutical compositions are suitable for administration to a human or non-human animal via any one or more route of administration using methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be routinely selected that are suitable for the mode of administration, solubility and/or stability desired or required.

The formulations described herein comprise active agents in a concentration resulting in a w/v appropriate for a desired dose. In certain aspects, the active agent is present in a formulation at a concentration of about 1 mg/ml to about 200 mg/ml, about 1 mg/ml to about 100 mg/ml, about 1 mg/ml to about 50 mg/ml, or 1 mg/ml and about 25 mg/ml. In certain aspects, the concentration of the active agent in a formulation may vary from about 0.1 to about 100 weight %. In certain aspects, the concentration of the active agent is in the range of 0.003 to 1.0 molar.

In one aspect, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). In certain specific aspects, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the disclosure should be sterile. The formulations of the disclosure may be sterilized by various sterilization methods, including sterile filtration, radiation, etc. In one aspect, the formulation is filter-sterilized with a presterilized 0.22-micron filter. Sterile compositions for injection can be formulated according to conventional pharmaceutical practice as described in “Remington: The Science & Practice of Pharmacy”, 21st ed., Lippincott Williams & Wilkins, (2005).

Therapeutic compositions of the present disclosure can be formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Formulations of the present disclosure which are suitable for topical or transdermal administration include powders, sprays, ointments, pastes, creams, lotions, gels, solutions, patches and inhalants. The iMers may be mixed under sterile conditions with a pharmaceutically acceptable carrier, and with any preservatives, buffers, or propellants which may be required (U.S. Pat. Nos. 7,378,110; 7,258,873; 7,135,180; US Publication No. 2004-0042972; and 2004-0042971).

The formulations may conveniently be presented in unit dosage form and may be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages may range from about 0.0001 to about 100 mg/kg of body weight or greater, for example about 0.1, 1, 10, or 50 mg/kg of body weight, with about 1 to about 10 mg/kg of body weight being suitable.

Note that the disclosure similarly contemplates that formulations suitable for diagnostic and research use may also be made. The concentration of active agent in such formulations, as well as the presence or absence of excipients and/or pyrogens can be selected based on the particular application and intended use.

### E. Uses

iMers, as described herein, may be used to bind targets associated with diseases or disorders, thereby removing or otherwise inhibiting the activity of the targets and treating the diseases or disorders and/or alleviating symptoms thereof. In some aspects, the iMers of the disclosure may be used for treating a disorder related to angiogenesis, cell proliferation, cell motility, cell invasion, or cell adhesion in a subject, by administering to the subject in need thereof a therapeutically effective dose of an iMer of the disclosure (which, in certain features, may be an iMab) that binds to a target associated with the disease or symptoms of the disorder. For example, aberrant signalling through growth factors and/or growth factor receptors has been shown to contribute to unwanted cell proliferation and cancer. Accordingly, iMers may be used to treat unwanted cell proliferation and/or cancer associated with growth factor signalling. In particular, the tumor growth curve of a tumor and/or the volume of a tumor may be reduced by administration of an iMer directed to proteins in growth factor signalling pathways. A similar strategy may be used for specific cancers or examples of unwanted cell proliferation associated with other signalling molecules.

An exemplary, non-limiting iMer, such as an iMab, is a bispecific molecule that binds EGFR and IGFR. This bispecific molecule may be used to treat unwanted cell proliferation and/or cancer associated with EGR and IGF signalling. For example, the bispecific antibody may be used to inhibit tumor growth and/or decrease the volume of an existing tumor.

In other aspects, the iMers of the disclosure may be used to treat diseases or disorders caused by an infectious agent such as a virus, bacteria, or parasite. An iMer may be designed to bind to one or more biomolecular targets on the agent, thereby rendering the agent unable to invade and/or infect the host cells. Alternately, a plurality of iMers may bind to an infectious agent and, like native immunoglobulins, trigger an immune response. Finally, an iMer may be used to bind a target molecule released by an infectious agent, in order to prevent the target from acting on host cells and tissues.

In still other aspects, iMers of the disclosure may be used to modulate immune responses to antigens such as pollen, plants, insect parts and/or secretions, animal dander, nuts, or self-antigens. iMers may be engineered to bind biomolecular targets present in these antigens and/or targets that mediate the immune response to these targets, like IgE, anaphylatoxins, or histamine.

iMers of the disclosure, such as those described herein, may also be used for diagnostic purposes. For example, one or more target biomolecules may be detected in tissues or cells of a subject in order to screen for a disease or disorder associated with changes in expression of the targets. A diagnostic kit may comprise one or more iMers that bind to target molecules, and a detection system for indicating the reaction of the iMer(s) with the target(s), if any.

In addition to therapeutic and diagnostic uses, the nucleic acid molecules and methods provided herein are useful in that they provide compositions and methods that facilitate efficient production of multimeric, multispecific polypeptides, such as iMers and iMabs. As described herein, one of the significant advantages provided by the present disclosure is the ability to efficiently produce iMers that, because of the construction of the nucleic acid molecules that encode the relevant polypeptides, do not require extensive, time consuming, laborious purification to remove contaminating homodimeric or other mismatched forms of the desired multimers.

In certain aspects, iMers (which include iMabs) of the present disclosure bind to different epitopes or sites on the same target molecules. In other aspects, iMers of the present disclosure bind to different sites on different target molecules. Regardless of whether the polypeptides are being used in a therapeutic, diagnostic, imaging, or research context, iMers bind at least 2 sites and, in other aspects, 3, 4, or more than 4 sites.

### F. Kits

Another aspect of the present disclosure is a kit. In one aspect, a kit comprises any of the compositions or pharmaceutical compositions of a nucleic acid, polypeptide, expression vector, or host cell described above, and instructions for use or administration. Optionally, the kit may comprise protease that can be added to cleave the linkers that interconnect the portions of the contiguous polypeptide. The disclosure contemplates that all or any subset of the components for conducting research assays, diagnostic assays and/or for administering therapeutically effective amounts may be enclosed in the kit. Similarly, the kit may include instructions for making a polypeptide by, for example culturing a host cell that expresses a nucleic acid that encodes an iMer of the disclosure (which includes, for example, an iMab of the disclosure) under suitable conditions. By way of additional example, a kit for therapeutic administration of an iMer of the disclosure may comprise a solution containing a pharmaceutical formulation of the iMer, or a lyophilized preparation of an iMer, and instructions for administering the composition to a patient in need thereof.

A kit for diagnostic assays may comprise a solution containing an iMer or a lyophilized preparation of an iMer of the disclosure, wherein the iMer binds specifically to one or more targets, as well as reagents for detecting such iMers. The iMers may be labeled according to methods known in the art and described herein, including but not limited to labels such as small molecule fluorescent tags, proteins such as biotin, GFP or other fluorescent proteins, or epitope sequences such as his or myc. Similarly, primary antibodies used for detecting iMers may be included in the kit. Primary antibodies may be directed to sequences on the iMers or to labels, tags, or epitopes with which the iMers are labeled. Primary antibodies may, in turn, be labeled for detection, or, if further amplification of the signal is desired, the primary antibodies may be detected by secondary antibodies, which may also be included in the kit.

### 7. SPECIFIC EMBODIMENTS

1. A nucleic acid molecule encoding a contiguous, multimeric polypeptide comprising at least two subunits, each of which includes at least a functional domain (FD) and an interaction domain (ID), wherein the nucleic acid molecule comprises:
i) a nucleic acid portion comprising a nucleotide sequence that encodes a functional domain (FD1) that binds to a first binding site;
ii) a nucleic acid portion comprising a nucleotide sequence that encodes an interaction domain (ID1);
iii) a nucleic acid portion comprising a nucleotide sequence that encodes a functional domain (FD2) that binds to a second binding site; and
iv) a nucleic acid portion comprising a nucleotide sequence that encodes an interaction domain (ID2);

wherein the nucleic acid molecule further comprises at least one nucleic acid portion comprising a nucleotide sequence encoding a polypeptide linker, which polypeptide linker includes at least one cleavage site, wherein ID1 and ID2 are capable of associating with each other, and wherein the contiguous, multimeric polypeptide is multispecific.

2. The nucleic acid molecule of embodiment 2, wherein the nucleic acid molecule comprises three nucleic acid portions, each of which comprise a nucleotide sequence that encodes a polypeptide linker.

3. The nucleic acid molecule of embodiment 2, wherein each of the polypeptide linkers is different.

4. The nucleic acid molecule of embodiment 2, wherein at least one of the polypeptide linkers is different.

5. The nucleic acid molecule of embodiment 1, wherein the nucleic acid portion comprising the nucleotide sequence that encodes the FD1 and the nucleic acid portion comprising the nucleotide sequence that encodes the ID1 comprises:
a) a nucleic acid segment comprising a nucleotide sequence that encodes an antibody light chain comprising a variable domain (VL1) and a light constant domain 1 (LCD1); and
b) a nucleic acid segment comprising a nucleotide sequence that encodes an antibody heavy chain comprising a variable domain (VH1) and a heavy constant domain 1 (HCD1);

wherein the nucleic acid portion comprising the nucleotide sequence that encodes the FD2 and the nucleic acid portion comprising the nucleotide sequence that encodes the ID2 comprises:
c) a nucleic acid segment comprising a nucleotide sequence that encodes an antibody light chain comprising a variable domain (VL2) and a light constant domain 2 (LCD2); and
d) a nucleic acid segment comprising a nucleotide sequence that encodes an antibody heavy chain comprising a variable domain (VH2) and a heavy constant domain 2 (HCD2);

wherein the contiguous polypeptide is a multispecific antibody.

6. The nucleic acid molecule of embodiment 5, wherein VL1 and VL2 are not the same antibody light chain variable domain and/or wherein VH1 and VH2 are not the same antibody heavy chain variable domain.

7. The nucleic acid molecule of embodiment 5 or 6, wherein the light constant domain 1 and the light constant domain 2 are not the same antibody light chain constant domain.

8. The nucleic acid molecule of embodiment 5, 6, or 7, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

9. The nucleic acid molecule of embodiment 8, wherein the heavy constant domain 1 comprises a C_{H}1 and C_{H}2 region, and wherein the heavy constant domain 2 comprises a C_{H}1 and C_{H}2 region.

10. The nucleic acid molecule of embodiment 8, wherein the heavy constant domain 1 comprises a C_{H}1, a C_{H}2, and a C_{H}3 region, and wherein the heavy constant domain 2 comprises a C_{H}1, a C_{H}2, and C_{H}3 region.

11. The nucleic acid molecule of any of embodiments 8-10, wherein the heavy constant domain 1 further comprises a hinge region, and wherein the heavy constant domain 2 further comprises a hinge region.

12. The nucleic acid molecule of any of embodiments 8-10, wherein the heavy constant domain 1 does not include a hinge region, and wherein the heavy constant domain 2 does not include a hinge region.

13. The nucleic acid molecule of any of embodiments 8-12, wherein the heavy constant domain 1 and the heavy constant domain 2 are the same.

14. The nucleic acid molecule of any of embodiments 8-12, wherein the heavy constant domain 1 and the heavy constant domain 2 are not the same.

15. The nucleic acid molecule of any of embodiments 1-15, wherein the at least one cleavage site comprises at least one protease cleavage site.

16. The nucleic acid molecule of any of embodiments 5-15, wherein the nucleic acid molecule comprises at least two nucleic acid segments each of which comprises a nucleotide sequence that encodes a polypeptide linker.

17. The nucleic acid molecule of any of embodiments 5-15, wherein the nucleic acid molecule comprises three nucleic acid segments each of which comprises a nucleotide sequence that encodes a polypeptide linker.

18. The nucleic acid molecule of embodiment 16 or 17, wherein at least one of the polypeptide linkers comprises at least one protease cleavage site.

19. The nucleic acid molecule of any of embodiments 16-18, wherein at least one of the polypeptide linkers comprises two protease cleavage sites.

20. The nucleic acid molecule of embodiment 17, wherein each of the three polypeptide linkers comprises at least one protease cleavage site.

21. The nucleic acid molecule of embodiment 20, wherein each of the three polypeptide linkers comprise two protease cleavage sites.

22. The nucleic acid molecule of any of embodiments 15-21, wherein each of the polypeptide linkers is different.

23. The nucleic acid molecule of any of embodiments 15-21, wherein at least one of the polypeptide linkers is different.

24. The nucleic acid molecule of any of embodiments 5-23, wherein the nucleic acid molecule comprises the nucleic acid segments in the following orientation from N-terminus to C-terminus:
i) the nucleic acid segment comprising the nucleotide sequence that encodes an antibody light chain comprising the variable domain (VL1) and the light constant domain 1 (LCD1);
ii) the nucleic acid segment comprising the nucleotide sequence that encodes an antibody heavy chain comprising the variable domain (VH1) and the heavy constant domain 1 (HCD1);
iii) the nucleic acid segment comprising the nucleotide sequence that encodes an antibody light chain comprising the variable domain (VL2) and the light constant domain 2 (LCD1); and
iv) the nucleic acid segment comprising the nucleotide sequence that encodes an antibody heavy chain comprising the variable domain (VH2) and the heavy constant domain 2 (HCD1).

25. The nucleic acid molecule of embodiment 24, wherein the nucleic acid segment of (i) is operably linked to the nucleic acid segment of (ii) via a nucleic acid segment comprising a nucleotide sequence that encodes a first polypeptide linker.

26. The nucleic acid molecule of embodiment 24 or 25, wherein the nucleic acid segment of (ii) is operably linked to the nucleic acid segment of (iii) via a nucleic acid segment comprising a nucleotide sequence that encodes a second polypeptide linker.

27. The nucleic acid molecule of any of embodiments 24-26, wherein the nucleic acid segment of (iii) is operably linked to the nucleic acid segment of (iv) via a nucleic acid segment comprising a nucleotide sequence that encodes a third polypeptide linker.

28. The nucleic acid molecule of any of embodiments 25-27, wherein the first polypeptide linker comprises at least one protease cleavage site and/or the second polypeptide linker comprises at least one protease cleavage site and/or the third polypeptide linker comprises at least one protease cleavage site.

29. The nucleic acid molecule of embodiment 28, wherein the first polypeptide linker comprises two protease cleavage sites.

30. The nucleic acid molecule of embodiment 28 or 29, wherein the second polypeptide linker comprises two protease cleavage sites.

31. The nucleic acid molecule of any of embodiments 28-30, wherein the third polypeptide linker comprises two protease cleavage sites.

32. The nucleic acid molecule of any of embodiments 26-31, wherein each of the polypeptide linkers is different.

33. The nucleic acid molecule of any of embodiments 26-31, wherein at least one of the polypeptide linkers is different.

34. The nucleic acid molecule of any of embodiments 15-33, wherein the at least one protease cleavage site comprises a thrombin cleavage site.

35. The nucleic acid molecule of any of embodiments 15-33, wherein the at least one protease cleavage site comprises a furin cleavage site.

36. The nucleic acid molecule of any of embodiments 1-3, 15, and 34-35, wherein the nucleic acid portion comprising the nucleotide sequence that encodes the ID1 and the nucleic acid portion comprising the nucleotide sequence that encodes the ID2 each comprises a nucleic acid segment comprising a nucleotide sequence that encodes a coil-coil dimerization motif

37. The nucleic acid molecule of embodiment 36, wherein each of the coil-coil dimerization motifs are the same.

38. The nucleic acid molecule of embodiment 36 or 37, wherein each of the coil-coil dimerization motifs are selected from the group consisting of a Geminin coil-coil motif, an HIV major homology region coil-coil motif, Saccharomyces cerevisiae Sir4p, a coil-coil motif from a transcription factor, a zinc finger domain, a viral coat protein, p53, and a leucine zipper.

39. The nucleic acid molecule of any of embodiments 1-3, 15, and 34-35, wherein:
a) the nucleic acid portion comprising the nucleotide sequence that encodes the ID1 comprises a nucleic acid segment comprising a nucleotide sequence that encodes a heavy constant domain 1; and
b) the nucleic acid portion comprising the nucleotide sequence that encodes the ID2 comprises a nucleic acid segment comprising a nucleotide sequence that encodes a heavy constant domain 2.

40. The nucleic acid molecule of embodiment 39, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

41. The nucleic acid molecule of embodiment 40, wherein the heavy constant domain 1 comprises a C_{H}1 and C_{H}2 region, and wherein the heavy constant domain 2 comprises a C_{H}1 and C_{H}2 region.

42. The nucleic acid molecule of embodiment 40, wherein the heavy constant domain 1 comprises a C_{H}1, a C_{H}2, and a C_{H}3 region, and wherein the heavy constant domain 2 comprises a C_{H}1, a C_{H}2, and a C_{H}3 region.

43. The nucleic acid molecule of any of embodiments 40-42, wherein the heavy constant domain 1 further comprises a hinge region, and wherein the heavy constant domain 2 further comprises a hinge region.

44. The nucleic acid molecule of any of embodiments 40-42, wherein the heavy constant domain 1 does not include a hinge region, and wherein the heavy constant domain 2 does not include a hinge region.

45. The nucleic acid molecule of any of embodiments 39-44, wherein the heavy constant domain 1 and the heavy constant domain 2 are the same.

46. The nucleic acid molecule of any of embodiments 39-44, wherein the heavy constant domain 1 and the heavy constant domain 2 are not the same.

47. The nucleic acid molecule of any of embodiments 1-3, 15, 18-21 and 36-46, wherein the nucleic acid portion comprising the nucleotide sequence that encodes the FD1 comprises a nucleic acid segment comprising a nucleotide sequence that encodes an antigen binding portion of an antibody.

48. The nucleic acid molecule of any of embodiments 1-3, 15, 18-21 and 36-47, wherein the nucleic acid portion comprising the nucleotide sequence that encodes the FD2 comprises a nucleic acid segment comprising a nucleotide sequence that encodes an antigen binding portion of an antibody.

49. The nucleic acid molecule of any of embodiments 1-3, 15, 18-21 and 36-48, wherein the nucleic acid portion comprising the nucleotide sequence that encodes the FD1 comprises a nucleic acid segment comprising a nucleotide portion that encodes an scFv.

50. The nucleic acid molecule of any of embodiments 1-3, 15, 18-21 and 36-49, wherein the nucleic acid portion comprising the nucleotide sequence that encodes the FD2 comprises a nucleic acid segment comprising a nucleotide portion that encodes an scFv.

51. The nucleic acid molecule of any of embodiments 1-3, 15, 18-21, 36-48 and 50, wherein the nucleic acid portion comprising the nucleotide sequence that encodes the FD1 comprises a nucleic acid segment comprising a nucleotide portion that encodes a diabody.

52. The nucleic acid molecule of any of embodiments 1-3, 15, 18-21, 36-49 and 51, wherein the nucleic acid portion comprising the nucleotide sequence that encodes the FD2 comprises a nucleic acid segment comprising a nucleotide portion that encodes a diabody.

53. The nucleic acid molecule of any of embodiments 1-3, 15, 18-21, 36-48, 50 and 52, wherein the nucleic acid portion comprising the nucleotide sequence that encodes the FD1 comprises a nucleic acid segment comprising a nucleotide portion that encodes a triabody.

54. The nucleic acid molecule of any of embodiments 1-3, 15, 18-21, 36-49, 51 and 53, wherein the nucleic acid portion comprising the nucleotide sequence that encodes the FD2 comprises a nucleic acid segment comprising a nucleotide portion that encodes a diabody.

55. The nucleic acid molecule of any of embodiments 1-3, 15, 18-21, 36-48, 50, 52 and 54, wherein the nucleic acid portion comprising the nucleotide sequence that encodes the FD1 comprises a nucleic acid segment comprising a nucleotide portion that encodes a tandem scFv.

56. The nucleic acid molecule of any of embodiments 1-3, 15, 18-21, 36-49, 51, 53 and 55, wherein the nucleic acid portion comprising the nucleotide sequence that encodes the FD2 comprises a nucleic acid segment comprising a nucleotide portion that encodes a tandem scFv.

57. The nucleic acid molecule of any of embodiments 1-3, 15, 18-21 and 49-56, wherein the nucleic acid portion comprising the nucleotide sequence that encodes the ID1 and the nucleic acid portion comprising the nucleotide sequence that encodes the ID2 each comprises a nucleic acid segment comprising a nucleotide sequence that encodes an Fc region.

58. The nucleic acid molecule of embodiment 57, wherein the Fc regions are the same.

59. The nucleic acid molecule of embodiment 57, wherein the Fc regions are not the same.

60. The nucleic acid molecule of any of embodiments 57-59, wherein at least one of the Fc regions comprises a variant Fc region.

61. The nucleic acid molecule of any of embodiments 1-3, 18-21, 36-46, 48, 50, 52, 54 and 56-60, wherein the nucleic acid portion comprising the nucleotide sequence that encodes the FD1 comprises a nucleic acid segment comprising a nucleotide sequence that encodes a ligand binding domain.

62. The nucleic acid molecule of any of embodiments 1-3, 18-21, 36-46, 48, 50, 52, 54 and 56-60, wherein the nucleic acid portion comprising the nucleotide sequence that encodes the FD1 comprises a nucleic acid segment comprising a nucleotide sequence that encodes a receptor binding domain.

63. The nucleic acid molecule of any of embodiments 1-3, 18-21, 36-47, 49, 51, 53, 55 and 57-61, wherein the nucleic acid portion comprising the nucleotide sequence that encodes the FD2 comprises a nucleic acid segment comprising a nucleotide sequence that encodes a ligand binding domain.

64. The nucleic acid molecule of any of embodiments 1-3, 18-21, 36-47, 49, 51, 53, 55, 57-60 and 62, wherein the nucleic acid portion comprising the nucleotide sequence that encodes the FD2 comprises a nucleic acid segment comprising a nucleotide sequence that encodes a receptor binding domain.

65. The nucleic acid molecule of any of embodiments 1-3, 18-21, 36-46, 48, 50, 52, 54 and 56-62, wherein the nucleic acid portion comprising the nucleotide sequence that encodes FD1 comprises a nucleic acid segment comprising a nucleotide sequence that encodes an antibody mimetic.

66. The nucleic acid molecule of any of embodiments 1-3, 18-21, 36-47, 49, 51, 53, 55, 57-60 and 63-64, wherein the nucleic acid portion comprising the nucleotide sequence that encodes FD2 comprises a nucleic acid segment comprising a nucleotide sequence that encodes an antibody mimetic.

67. The nucleic acid molecule of any of embodiments 1-3 and 36-66, wherein the nucleic acid molecule comprises at least two nucleic acid segments each of which comprises a nucleotide sequence that encodes a polypeptide linker.

68. The nucleic acid molecule of embodiment 67, wherein the nucleic acid molecule comprises three nucleic acid segments each of which comprises a nucleotide sequence that encodes a polypeptide linker.

69. The nucleic acid molecule of embodiment 67 or 68, wherein at least one of the polypeptide linkers comprises at least one protease cleavage site.

70. The nucleic acid molecule of any of embodiments 67-69, wherein at least one of the polypeptide linkers comprises two protease cleavage sites.

71. The nucleic acid molecule of embodiment 68, wherein each of the three polypeptide linkers comprises at least one protease cleavage site.

72. The nucleic acid molecule of embodiment 71, wherein each of the three polypeptide linkers comprises two protease cleavage sites.

73. The nucleic acid molecule of any of embodiments 67-72, wherein each of the polypeptide linkers is different.

74. The nucleic acid molecule of any of embodiments 67-72, wherein at least one of the polypeptide linkers is different.

75. An expression vector comprising the nucleic acid molecule of any of embodiments 1-74 operably linked to a promoter.

76. A host cell comprising the expression vector of embodiment 75, and which host cell expresses the contiguous polypeptide.

77. A method of producing a contiguous polypeptide, comprising:
i) providing a nucleic acid molecule of any of embodiments 1-74; and
ii) expressing the nucleic acid molecule in a host cell.

78. A method of producing a contiguous polypeptide, comprising:
i) providing a host cell of embodiment 76 in a culture media suitable for growth of the cell and production of the polypeptide; and
ii) purifying the polypeptide from the cell or culture media.

79. The method of embodiment 78, further comprising adding, either before or after the step of purifying the polypeptide from the cell or culture media, a protease that cleaves the at least one protease cleavage site.

80. A nucleic acid molecule encoding a contiguous polypeptide, wherein the nucleic acid molecule comprises:
i) a nucleic acid segment comprising a nucleotide sequence that encodes an antibody light chain comprising a variable domain (VL1) and a light constant domain 1;
ii) a nucleic acid segment comprising a nucleotide sequence that encodes a first polypeptide linker;
iii) a nucleic acid segment comprising a nucleotide sequence that encodes an antibody heavy chain comprising a variable domain (VH1) and a heavy constant domain 1;
iv) a nucleic acid segment comprising a nucleotide sequence that encodes a second polypeptide linker;
v) a nucleic acid segment comprising a nucleotide sequence that encodes an antibody light chain comprising a variable domain (VL2) and a light constant domain 2;
vi) a nucleic acid segment comprising a nucleotide sequence that encodes a third polypeptide linker; and
vii) a nucleic acid segment comprising a nucleotide sequence that encodes an antibody heavy chain comprising a variable domain (VH2) and a heavy constant domain 2;

wherein the contiguous polypeptide is a multispecific polypeptide.

81. The nucleic acid molecule of embodiment 80, wherein VL1 and VL2 are not the same antibody light chain variable domain and/or wherein VH1 and VH2 are not the same antibody heavy chain variable domain.

82. The nucleic acid molecule of embodiment 80 or 81, wherein the light constant domain 1 and the light constant domain 2 are not the same antibody light chain constant domain.

83. The nucleic acid molecule of embodiment 80, 81, or 82, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

84. The nucleic acid molecule of embodiment 83, wherein the heavy constant domain 1 comprises a C_{H}1 and C_{H}2 region, and wherein the heavy constant domain 2 comprises a C_{H}1 and C_{H}2 region.

85. The nucleic acid molecule of embodiment 83, wherein the heavy constant domain 1 comprises a C_{H}1, a C_{H}2, and a C_{H}3 region, and wherein the heavy constant domain 2 comprises a C_{H}1, a C_{H}2, and C_{H}3 region.

86. The nucleic acid molecule of any of embodiments 83-85, wherein the heavy constant domain 1 further comprises a hinge region, and wherein the heavy constant domain 2 further comprises a hinge region.

87. The nucleic acid molecule of any of embodiments 83-85, wherein the heavy constant domain 1 does not include a hinge region, and wherein the heavy constant domain 2 does not include a hinge region.

88. The nucleic acid molecule of any of embodiments 80-87, wherein the heavy constant domain 1 and the heavy constant domain 2 are the same.

89. The nucleic acid molecule of any of embodiments 80-87, wherein the heavy constant domain 1 and the heavy constant domain 2 are not the same.

90. The nucleic acid molecule of any of embodiments 80-89, wherein the first polypeptide linker comprises at least one protease cleavage site.

91. The nucleic acid molecule of any of embodiments 80-90, wherein the second polypeptide linker comprises at least one protease cleavage site.

92. The nucleic acid molecule of any of embodiments 80-91, wherein the third polypeptide linker comprises at least one protease cleavage site.

93. The nucleic acid molecule of any of embodiments 90-92, wherein at least one of the polypeptide linkers comprises two protease cleavage sites.

94. The nucleic acid molecule of embodiment 90-93, wherein each of the three polypeptide linkers comprises at least one protease cleavage site.

95. The nucleic acid molecule of embodiment 93 or 94, wherein each of the three polypeptide linkers comprise two protease cleavage sites.

96. The nucleic acid molecule of any of embodiments 80-95, wherein each of the polypeptide linkers is different.

97. The nucleic acid molecule of any of embodiments 80-95, wherein at least one of the polypeptide linkers is different.

98. An expression vector comprising the nucleic acid molecule of any of embodiments 80-97 operably linked to a promoter.

99. A host cell comprising the expression vector of embodiment 98, and which host cell expresses the contiguous, multimeric polypeptide.

100. A method of producing a contiguous polypeptide, comprising:
i) providing a nucleic acid molecule of any of embodiments 80-97; and
ii) expressing the nucleic acid molecule in a host cell.

101. A method of producing a contiguous polypeptide, comprising
i) providing a host cell of embodiment 99 in a culture media suitable for growth of the cell and production of the polypeptide and
ii) purifying the polypeptide from the cell or culture media.

102. A nucleic acid molecule encoding a contiguous polypeptide, wherein the nucleic acid molecule comprises:
i) a nucleic acid segment comprising a nucleotide sequence that encodes an antibody light chain comprising a variable domain (VL1) and a light constant domain 1;
ii) a nucleic acid segment comprising a nucleotide sequence that encodes an antibody heavy chain comprising a variable domain (VH1) and a heavy constant domain 1; and
iii) a nucleic acid segment comprising a nucleotide sequence that encodes an antibody heavy chain comprising a heavy constant domain 2;

wherein the contiguous polypeptide is an antibody that binds one or more epitopes.

103. A nucleic acid molecule encoding a contiguous polypeptide, wherein the nucleic acid molecule comprises:
i) a nucleic acid segment comprising a nucleotide sequence that encodes an antibody light chain comprising a variable domain (VL1) and a light constant domain 1;
ii) a nucleic acid segment comprising a nucleotide sequence that encodes a first polypeptide linker;
iii) a nucleic acid segment comprising a nucleotide sequence that encodes an antibody heavy chain comprising a variable domain (VH1) and a heavy constant domain 1;
iv) a nucleic acid segment comprising a nucleotide sequence that encodes a second polypeptide linker;
v) a nucleic acid segment comprising a nucleotide sequence that encodes an antibody heavy chain comprising a heavy constant domain 2;

wherein the contiguous polypeptide is an antibody that binds one or more epitopes.

104. The nucleic acid molecule of embodiment 103, wherein the first polypeptide linker comprises at least one protease cleavage site and/or the second polypeptide linker comprises at least one protease cleavage site.

105. The nucleic acid molecule of embodiment 104, wherein the first polypeptide linker comprises two protease cleavage sites and the second polypeptide linker comprises two protease cleavage sites.

106. The nucleic acid molecule of any of embodiments 103-105, wherein each of the polypeptide linkers is different.

107. The nucleic acid molecule of any of embodiments 103-105, wherein each of the polypeptide linkers is the same.

108. The nucleic acid molecule of any of embodiments 103-107, wherein the nucleic acid molecule comprises the nucleic acid segments in the following orientation from N-terminus to C-terminus
i) the nucleic acid segment comprising the nucleotide sequence that encodes an antibody light chain comprising the variable domain (VL1) and the light constant domain 1;
ii) the nucleic acid segment comprising the nucleotide sequence that encodes the first polypeptide linker;
iii) the nucleic acid segment comprising the nucleotide sequence that encodes an antibody heavy chain comprising the variable domain (VH1) and the heavy constant domain 1;
iv) the nucleic acid segment comprising the nucleotide sequence that encodes the second polypeptide linker; and
v) the nucleic acid segment comprising the nucleotide sequence that encodes an antibody heavy chain comprising the heavy constant domain 2.

109. An expression vector comprising the nucleic acid molecule of any of embodiments 102-108 operably linked to a promoter.

110. A host cell comprising the expression vector of embodiment 109, and which host cell expresses the contiguous, multimeric polypeptide.

111. A method of producing a contiguous polypeptide, comprising:
i) providing a nucleic acid molecule of any of embodiments 102-108; and
ii) expressing the nucleic acid molecule in a host cell.

112. A method of producing a contiguous polypeptide, comprising:
i) providing a host cell of embodiment 110 in a culture media suitable for growth of the cell and production of the polypeptide and
ii) purifying the polypeptide from the cell or culture media.

113. A multimeric polypeptide comprising at least two subunits, each of which includes at least a functional domain (FD) and an interaction domain (ID), and which polypeptide comprises an amino acid sequence with the formula: FD1-ID1-FD2-ID2, wherein
FD 1 comprises a first functional domain that binds to a first binding site;
ID1 comprises a first interaction domain;
FD2 comprises a second functional domain that binds to a second binding site; and
ID2 comprises a second interaction domain;

wherein the first interaction domain and the second interaction domain are capable of associating with each other, wherein the polypeptide is a single polypeptide chain when examined under reducing and/or denaturing conditions, and

wherein the multimeric polypeptide further comprises at least one polypeptide linker, which polypeptide linker includes at least one cleavage site.

114. The polypeptide of embodiment 113, wherein the at least one cleavage site is a protease cleavage site.

115. The polypeptide of embodiment 113 or 114, wherein the multimeric polypeptide comprises at least two polypeptide linkers or at least three polypeptide linkers.

116. The polypeptide of embodiment 113-115, wherein each of the polypeptide linkers includes at least one protease cleavage site.

117. The polypeptide of embodiment 113-114, wherein
FD1-ID1 comprises an amino acid sequence with the formula VL1-(light constant domain 1)-VH1-(heavy constant domain 1); and
FD2-ID2 comprises an amino acid sequence with the formula VL2-(light constant domain 2)-VH2-(heavy constant domain 2),

wherein VL1 comprises a first light chain variable domain; light constant domain 1 comprises a first light chain constant domain; VH1 comprises a first heavy chain variable domain; heavy constant domain 2 comprises a first heavy chain constant domain, which VL1 domain and VH1 domain correspond to FD1 and bind to the first binding site; and

wherein VL2 comprises a second light chain variable domain; light constant domain 2 comprises a second light chain constant domain; VH2 comprises a second heavy chain variable domain; heavy constant domain 2 comprises a second heavy chain constant domain, which VL2 domain and VH2 domain correspond to FD2 and bind to the second binding site.

118. The polypeptide of embodiment 117, comprising a polypeptide linker that interconnects the light constant domain 1 to the VH1 domain and/or a polypeptide linker that interconnects the heavy constant domain 1 to the VL2 domain and/or a polypeptide linker that interconnects the light constant domain 2 to the VH2 domain.

119. The polypeptide of embodiment 118, comprising a polypeptide linker that interconnects the light constant domain 1 to the VH1 domain and a polypeptide linker that interconnects the heavy constant domain 1 to the VL2 domain and a polypeptide linker that interconnects the light constant domain 2 to the VH2 domain.

120. The polypeptide of embodiment 119, wherein the polypeptide linker that interconnects the light constant domain 1 to the VH1 domain includes at least one protease cleavage site and the polypeptide linker that interconnects the heavy constant domain 1 to the VL2 domain includes at least one protease cleavage site and the polypeptide linker that interconnects the light constant domain 2 to the VH2 domain includes at least one protease cleavage site.

121. The polypeptide of any of embodiments 117-120, wherein VL1 and VL2 are not the same antibody light chain variable domain and/or wherein VH1 and VH2 are not the same antibody heavy chain variable domain.

122. The polypeptide of any of embodiments 117-120, wherein the light constant domain 1 and the light constant domain 2 are not the same antibody light chain constant domain.

123. The polypeptide of any of embodiments 117-121, wherein the heavy constant domain 1 comprises one or more of a CH1, CH2, and CH3 regions, and wherein the heavy constant domain 2 comprises one or more of a CH1, CH2, and CH3 regions.

124. The polypeptide of embodiment 123, wherein the heavy constant domain 1 comprises a C_{H}1 and a C_{H}2 region, and wherein the heavy constant domain 2 comprises a C_{H}1 and C_{H}2 region.

125. The polypeptide of embodiment 123, wherein the heavy constant domain 1 comprises a C_{H}1, a C_{H}2, and a C_{H}3 region, and wherein the heavy constant domain 2 comprises a C_{H}1, a C_{H}2, and a C_{H}3 region.

126. The polypeptide of any of embodiments 123-125, wherein the heavy constant domain 1 further comprises a hinge region, and wherein the heavy constant domain 2 further comprises a hinge region.

127. The polypeptide of any of embodiments 123-125, wherein the heavy constant domain 1 does not include a hinge region, and wherein the heavy constant domain 2 does not include a hinge region.

128. A polypeptide comprising an amino acid sequence with the formula:
VL1-(light constant domain 1)-VH1-(heavy constant domain 1)-VL2-(light constant domain 2)-VH2-(heavy constant domain 2),

wherein VL1 comprises a first light chain variable domain; light constant domain 1 comprises a first light chain constant domain; VH1 comprises a first heavy chain variable domain; heavy constant domain 1 comprises a first heavy chain constant domain, which VL1 domain and VH1 domain immunospecifically bind to a first epitope; and

wherein VL2 comprises a second light chain variable domain; light constant domain 2 comprises a second light chain constant domain; VH2 comprises a second heavy chain variable domain; heavy constant domain 2 comprises a second heavy chain constant domain, which VL2 domain and VH2 domain immunospecifically bind to a second epitope;

wherein the polypeptide is a single polypeptide chain when examined under reducing and/or denaturing conditions.

129. The polypeptide of embodiment 128, comprising a polypeptide linker that interconnects the light constant domain 1 to the VH1 domain and/or a polypeptide linker that interconnects the heavy constant domain 1 to the VL2 domain and/or a polypeptide linker that interconnects the light constant domain 2 to the VH2 domain.

130. The polypeptide of embodiment 128, comprising a polypeptide linker that interconnects the light constant domain 1 to the VH1 domain and a polypeptide linker that interconnects the heavy constant domain 1 to the VL2 domain and a polypeptide linker that interconnects the light constant domain 2 to the VH2 domain.

131. The polypeptide of embodiment 130, wherein the polypeptide linker that interconnects the light constant domain 1 to the VH1 domain includes at least one protease cleavage site and the polypeptide linker that interconnects the heavy constant domain 1 to the VL2 domain includes at least one protease cleavage site and the polypeptide linker that interconnects the light constant domain 2 to the VH2 domain includes at least one protease cleavage site.

132. The polypeptide of embodiment 131, wherein the polypeptide linker that interconnects the light constant domain 1 to the VH1 domain includes two protease cleavage site and the polypeptide linker that interconnects the heavy constant domain 1 to the VL2 domain includes two protease cleavage site and the polypeptide linker that interconnects the light constant domain 2 to the VH2 domain includes two protease cleavage site.

133. The polypeptide of any of embodiments 128-132, wherein VL1 and VL2 are not the same antibody light chain variable domain and/or wherein VH1 and VH2 are not the same antibody heavy chain variable domain.

134. The polypeptide of any of embodiments 128-132, wherein the light constant domain 1 and the light constant domain 2 are not the same antibody light chain constant domain.

135. The polypeptide of any of embodiments 128-134, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

136. The polypeptide of embodiment 135, wherein the heavy constant domain 1 comprises a C_{H}1 and a C_{H}2 region, and wherein the heavy constant domain 2 comprises a C_{H}1 and C_{H}2 region.

137. The polypeptide of embodiment 135, wherein the heavy constant domain 1 comprises a C_{H}1, a C_{H}2, and a C_{H}3 region, and wherein the heavy constant domain 2 comprises a C_{H}1, a C_{H}2, and a C_{H}3 region.

138. The polypeptide of any of embodiments 135-137, wherein the heavy constant domain 1 further comprises a hinge region, and wherein the heavy constant domain 2 further comprises a hinge region.

139. The polypeptide of any of embodiments 135-137, wherein the heavy constant domain 1 does not include a hinge region, and wherein the heavy constant domain 2 does not include a hinge region.

140. The polypeptide of any of embodiments 113-116, wherein ID1 comprises a coil-coil dimerization motif and ID2 comprises a coil-coil dimerization motif

141. The polypeptide of embodiment 140, wherein each of the coil-coil dimerization motifs are the same.

142. The polypeptide of embodiment 140 or 141, wherein each of the coil-coil dimerization motifs are selected from the group consisting of a Geminin coil-coil motif, an HIV major homology region coil-coil motif, Saccharomyces cerevisiae Sir4p, a coil-coil motif from a transcription factor, a zinc finger domain, a viral coat protein, p53, and a leucine zipper.

143. The polypeptide of any of embodiments 113-116, wherein ID1 comprises a heavy constant domain 1 and ID2 comprises a heavy constant domain 2.

144. The polypeptide of embodiment 143, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

145. The polypeptide of embodiment 144, wherein the heavy constant domain 1 comprises a C_{H}1 and C_{H}2 region, and wherein the heavy constant domain 2 comprises a C_{H}1 and C_{H}2 region.

146. The polypeptide of embodiment 144, wherein the heavy constant domain 1 comprises a C_{H}1, a C_{H}2, and a C_{H}3 region, and wherein the heavy constant domain 2 comprises a C_{H}1, a C_{H}2, and a C_{H}3 region.

147. The polypeptide of any of embodiments 144-146, wherein the heavy constant domain 1 further comprises a hinge region, and wherein the heavy constant domain 2 further comprises a hinge region.

148. The polypeptide of any of embodiments 144-146, wherein the heavy constant domain 1 does not include a hinge region, and wherein the heavy constant domain 2 does not include a hinge region.

149. The polypeptide of any of embodiments 143-148, wherein the heavy constant domain 1 and the heavy constant domain 2 are the same.

150. The polypeptide of any of embodiments 143-148, wherein the heavy constant domain 1 and the heavy constant domain 2 are not the same.

151. The polypeptide of any of embodiments 113-115, wherein ID1 and ID2 each comprise an Fc region.

152. The polypeptide of embodiment 151, wherein the Fc regions are the same.

153. The polypeptide of embodiment 151, wherein the Fc regions are not the same.

154. The polypeptide of any of embodiments 151-153, wherein at least one of the Fc regions comprises a variant Fc region.

155. The polypeptide of any of embodiments 113-116 and 140-154, wherein FD1 comprises an antigen binding portion of an antibody.

156. The polypeptide of any of embodiments 113-116 and 140-155, wherein FD2 comprises an antigen binding portion of an antibody.

157. The polypeptide of any of embodiments 113-116, 140-154 and 156, wherein FD1 comprises a ligand binding domain.

158. The polypeptide of any of embodiments 113-116, 140-154 and 156, wherein FD1 comprises a receptor binding domain.

159. The polypeptide of any of embodiments 113-116, 140-155 and 157-158, wherein FD2 comprises a ligand binding domain.

160. The polypeptide of any of embodiments 113-116, 140-155 and 157-158, wherein FD2 comprises a receptor binding domain.

161. The polypeptide of any of embodiments 113-116, 140-154, 156, 159 and 160, wherein the FD1 comprises an scFv.

162. The polypeptide of any of embodiments 113-116, 140-155, 157-158 and 161, wherein the FD2 comprises an scFv.

163. The polypeptide of any of embodiments 113-116, 140-154, 156, 159-160 and 162, wherein the FD1 comprises a diabody.

164. The polypeptide of any of embodiments 113-116, 140-155, 157-158, 161 and 163, wherein the FD2 comprises a diabody.

165. The polypeptide of any of embodiments 113-116, 140-154, 156, 159-160, 162 and 164, wherein the FD1 comprises a triabody.

166. The polypeptide of any of embodiments 113-116, 140-155, 157-158, 161, 163 and 165, wherein the FD2 comprises a diabody.

167. The polypeptide of any of embodiments 113-116, 140-154, 156, 159-160, 162, 164 and 166, wherein the FD1 comprises a tandem scFv.

168. The polypeptide of any of embodiments 113-116, 140-155, 157-158, 161, 163, 165 and 167, wherein the FD2 comprises a tandem scFv.

169. The polypeptide of any of embodiments 140-168, wherein the polypeptide comprises three polypeptide linkers.

170. The polypeptide of embodiment 169, wherein at least one of the polypeptide linkers comprises at least one protease cleavage site.

171. The polypeptide of embodiment 170, wherein at least one of the polypeptide linkers comprises two protease cleavage sites.

172. The polypeptide of embodiment 170, wherein each of the three polypeptide linkers comprises at least one protease cleavage site.

173. The polypeptide of embodiment 172, wherein each of the three polypeptide linkers comprises two protease cleavage sites.

174. The polypeptide of any of embodiments 169-173, wherein each of the polypeptide linkers is different.

175. The polypeptide of any of embodiments 169-173, wherein at least one of the polypeptide linkers is different.

### 8. EXAMPLES

The disclosure is now described with reference to the following examples. These examples are provided for the purpose of illustration only and the disclosure should in no way be construed as being limited to these examples but rather should be construed to encompass any and all variations which become evident as a result of the teachings provided herein. In general terms, the present disclosure employs, unless otherwise indicated, conventional techniques of molecular biology, chemistry, biochemistry, biophysics, recombinant DNA technology, immunology, and antibody engineering. All these techniques are of common knowledge to somebody working in the same field. Of course, it will be appreciated that specific listing or description of particular equipment and reagents used, sizes, manufacturer, etc., is not to be considered limiting on the current disclosure unless specifically stated to be so. It with be further appreciated that other equipment and reagents which perform similarly may be readily substituted.

### Example 1

### Expression, Purification and SDS-PAGE Analysis of a Monovalent Bispecific iMer

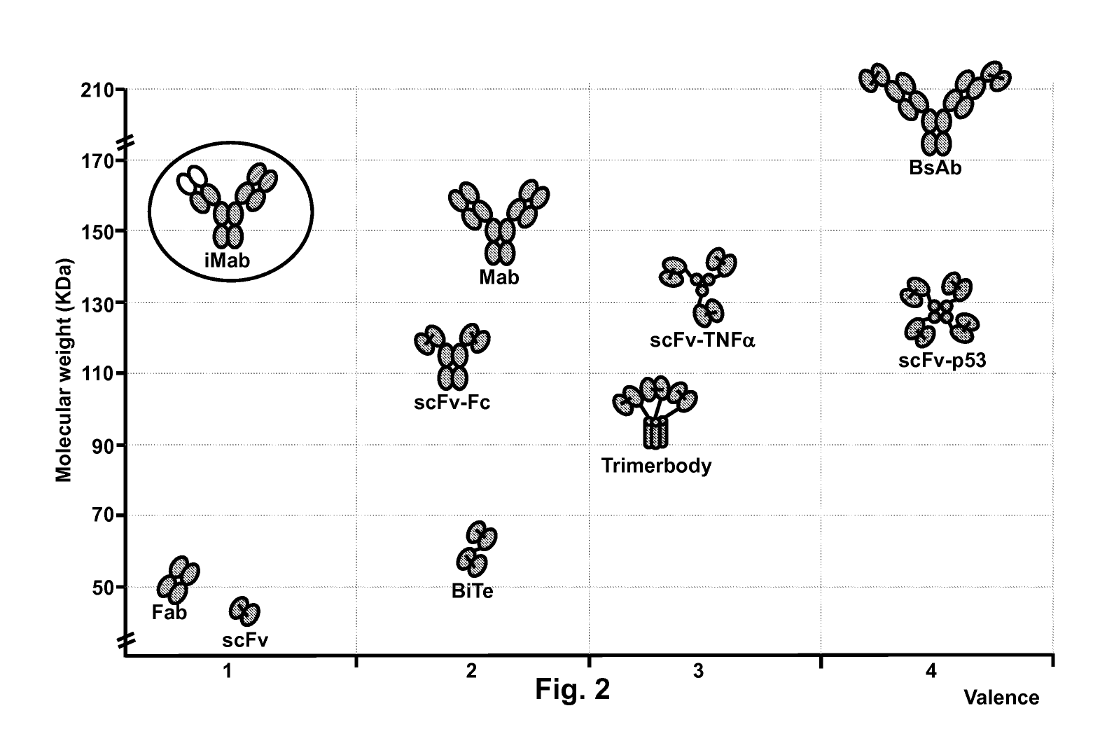

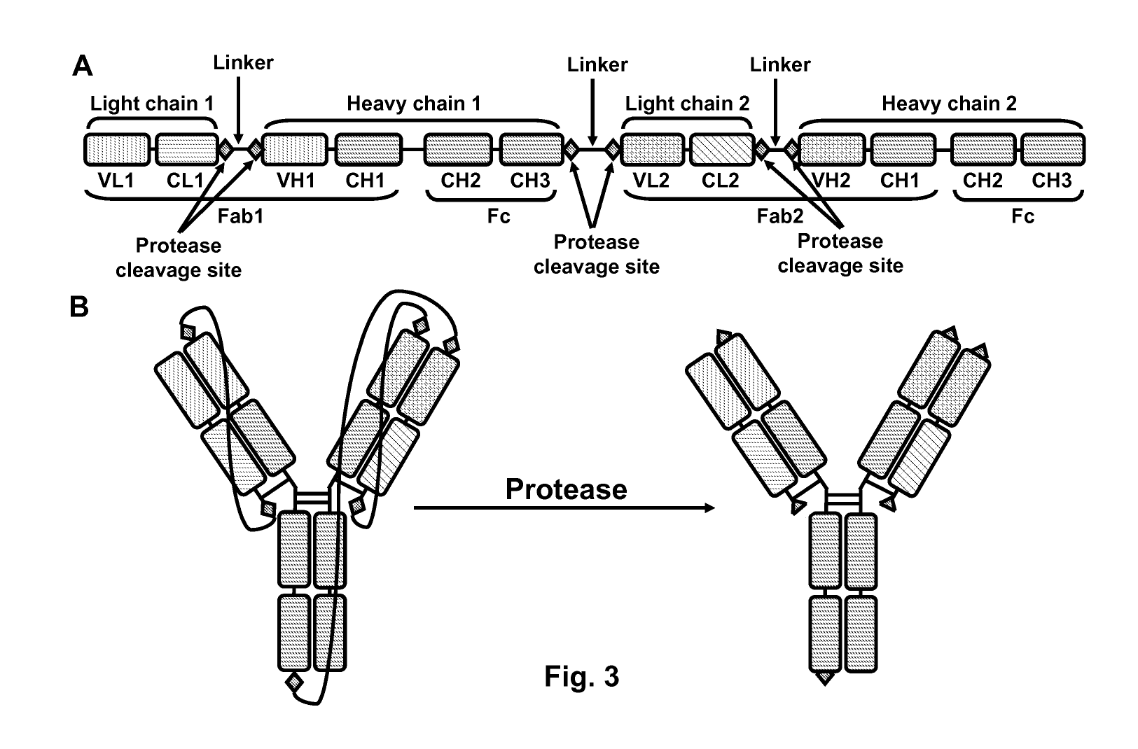

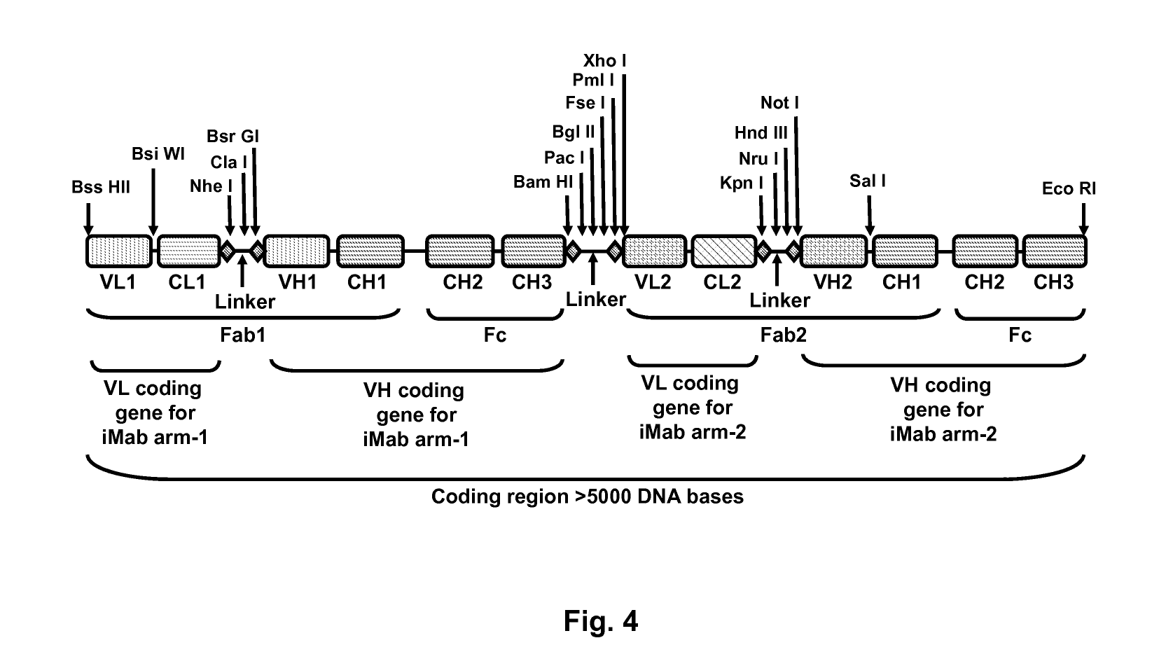

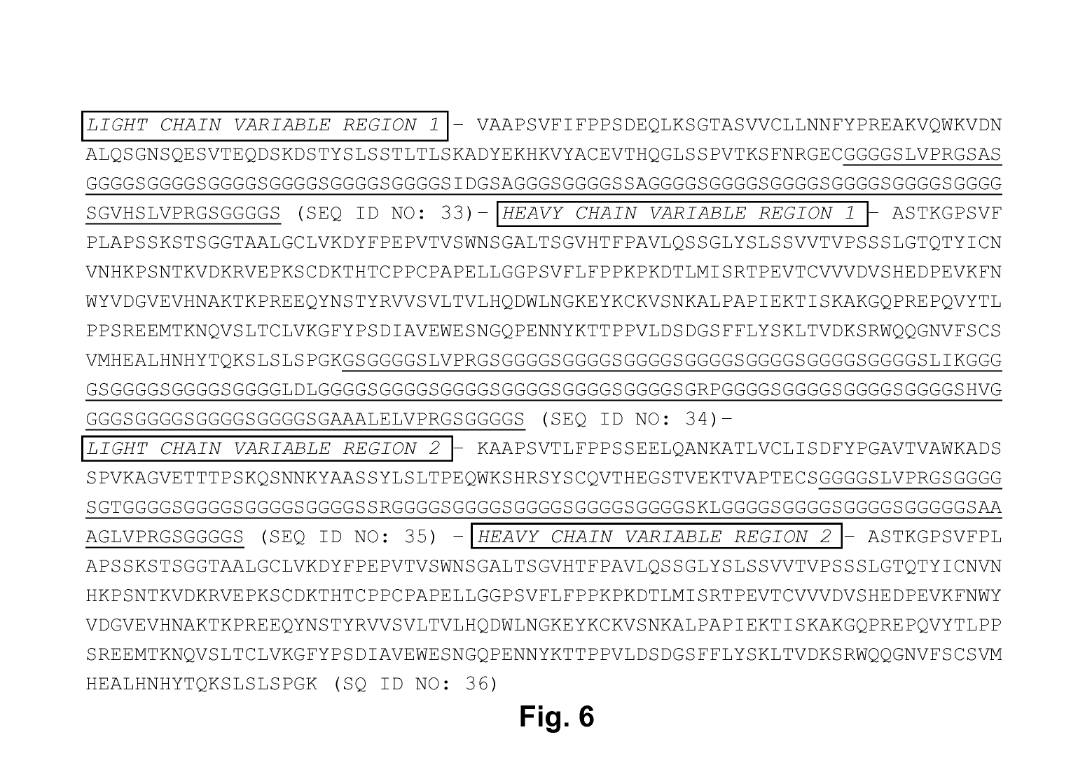

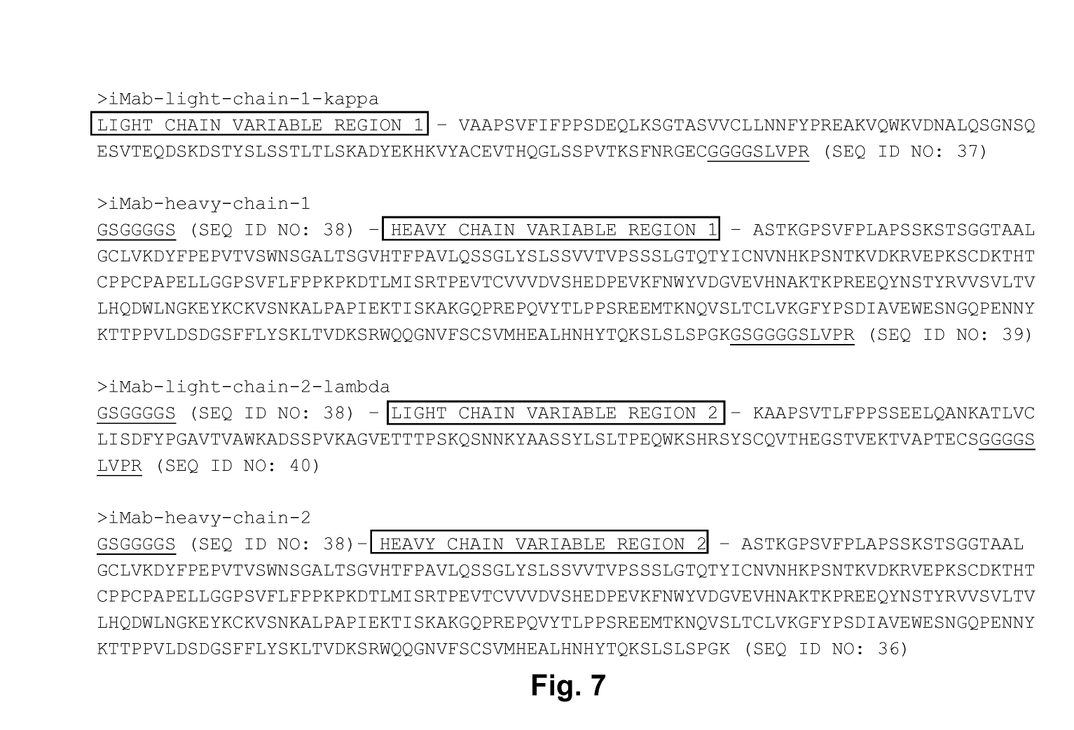

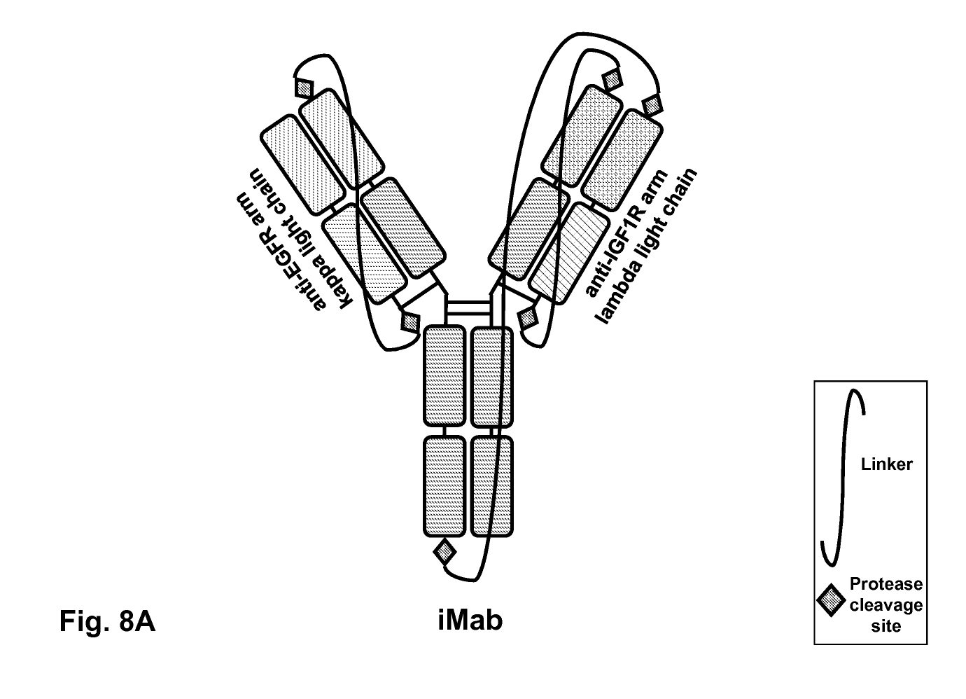

FIG. 1 depicts a representative monovalent, bispecific iMer that has a domain organization that is comparable to a conventional antibody (see FIG. 2 for a comparison of valence and molecular weight of a number of antibody formats). This monovalent bispecific iMer format is, in certain aspects, more specifically referred to herein as an iMab. As shown in FIG. 3, the individual domains of the iMab are expressed as a contiguous polypeptide chain connected by linkers. A protease site at the beginning and end of each linker allows for their removal by protease digestion. A DNA encoding the representative iMab used in the examples below is depicted schematically in FIG. 4. FIGS. 5 and 6 provide the nucleotide and amino acid sequences, respectively, of the constant regions and the linkers of a DNA encoding a representative iMab, the variable regions are represented by boxed text and may be any desired combination of variable regions. Table 1 provides amino acid and corresponding nucleotide sequences of variable regions that bind EGFR and IGF-1R which may be utilized to generate the iMab-EI described below. In this figure the linkers encode a Thrombin cleavage site, however, any desired cleavage site could be utilized. FIG. 7 shows the amino acid sequences of the constant regions and remaining linker portions for the heavy and light chains after protease digestion (Thrombin in this example). Additional representative iMab and iMer constructs are presented in FIGS. 22-29. As with the iMab presented in FIG. 1, each iMab or iMer construct can be expressed as a contiguous polypeptide chain connected by linkers. It is specifically contemplated that a protease site may be present, for example at the beginning and end of each linker, to facilitate linker removal by protease digestion.

A DNA encoding an iMab (incorporating SEQ ID NOs: 1, 3, 13 and 15) referred to herein as iMab-EI as depicted in FIG. 8A, was transfected in HEK293F and CHO cells using standard protocols. The transfected cells were cultivated for 10 days in Invitrogen's Freestyle™ media. Recombinant expression was determined using a protein A binding assay. Briefly, the culture media was automatically loaded onto a protein A column using an HPLC system (Agilent 1100 Capillary LC System, Foster City, Calif.). Unbound material was washed with a solution of 100 mM sodium phosphate buffer at pH 6.8, and antibodies were eluted with 0.1% phosphoric acid, pH 1.8. The area corresponding to the eluted peak was integrated and the total antibody concentration was determined by comparing to an immunoglobulin standard. The concentrations of the purified constructs were also determined by reading the absorbance at 280 nm using theoretically determined extinction coefficients. The iMab-EI was affinity purified using standard protein A affinity chromatography using HiTrap rProteinA FF column that have been equilibrated in PBS 1×. The iMab-EI was loaded on the column and after loading the column was washed, to eliminate contaminants and unbound material, with PBS 1× until the A280 trace reached baseline. The bound protein was then eluted in 25 mM glycine pH 2.8. Fractions were immediately neutralized by addition of 0.1 volumes 1M Tris-HCl buffer pH 8. Fractions were analyzed for their iMab-EI content by reading their absorbance at A280. The fraction containing the iMab-EI were pooled together and dialyzed overnight using a dialysis membrane cutoff of 10,000 kiloDalton (kDa), at 4° C. in 10× volume of PBS 1×. The dialyzed iMab-EI was then filtered using 0.22 micron filters and analyzed by reducing and non-reducing SDS-PAGE (FIG. 8B). The SDS-PAGE used was a 4-12% Nupage gel run in MOPS buffer. Approximately 3 micrograms of iMab-EI or parental arm antibodies, as shown in FIG. 3, were loaded on the SDS-PAGE. As shown in FIG. 8B, the iMab-EI has the expected molecular mass and has the expected SDS-PAGE molecular patterns. Similar results were seen with iMabs incorporating other variable regions and/or comprising linkers having different protease cleavage sites (Example 14 and data not shown).

TABLE 1
VH and VL amino acid and nucleotide sequences

| Target | VH | VL |
|---|---|---|
| EGFR | QVQLQESGPGLVKPSETLSLTCTVS | DIQMTQSPSSLSASVGDRVTITCQAS |
| | GGSVSSGDYYWTWIRQSPGKGLEW | QDISNYLNWYQQKPGKAPKLLIYDA |
| | IGHIYYSGNTNYNPSLKSRLTISIDTS | SNLETGVPSRFSGSGSGTDFTFTISSL |
| | KTQFSLKLSSVTAADTAIYYCVRDR | QPEDIATYFCQHFDHLPLAFGGGTKV |
| | VTGAFDIWGQGTMVTVSSA (SEQ | EIK (SEQ ID NO: 3) |
| | ID NO: 1) | |
| | CAAGTTCAACTTCAAGAATCTGGT | GATATTCAAATGACTCAATCTCCTT |
| | CCTGGTCTTGTTAAACCTTCTGAA | CTTCTCTTTCTGCTTCTGTTGGTGAT |
| | ACTCTTTCTCTTACTTGTACTGTTT | CGTGTTACTATTACTTGTCAAGCTT |
| | CTGGTGGTTCTGTTTCTTCTGGTG | CTCAAGATATTTCTAATTATCTTAA |
| | ATTATTATTGGACTTGGATTCGTC | TTGGTATCAACAAAAACCTGGTAA |
| | AATCTCCTGGTAAAGGTCTTGAAT | AGCTCCTAAACTTCTTATTTATGAT |
| | GGATTGGTCATATTTATTATTCTG | GCTTCTAATCTTGAAACTGGTGTTC |
| | GTAATACTAATTATAATCCTTCTC | CTTCTCGTTTTTCTGGTTCTGGTTCT |
| | TTAAATCTCGTCTTACTATTTCTAT | GGTACTGATTTTACTTTTACTATTTC |
| | TGATACTTCTAAAACTCAATTTTC | TTCTCTTCAACCTGAAGATATTGCT |
| | TCTTAAACTTTCTTCTGTTACTGCT | ACTTATTTTTGTCAACATTTTGATCA |
| | GCTGATACTGCTATTTATTATTGT | TCTTCCTCTTGCTTTTGGTGGTGGTA |
| | GTTCGTGATCGTGTTACTGGTGCT | CTAAAGTTGAAATTAAA (SEQ ID |
| | TTTGATATTTGGGGTCAAGGTACT | NO: 4) |
| | ATGGTTACTGTTTCTTCTGCT (SEQ | |
| | ID NO: 2) | |
| EGFR | QVQLKQSGPGLVQPSQSLSITCTVS | DILLTQSPVILSVSPGERVSFSCRASQS |
| | GFSLTNYGVHWVRQSPGKGLEWL | IGTNIHWYQQRTNGSPRLLIKYASESI |
| | GVIWSGGNTDYNTPFTSRLSINKDN | SGIPSRFSGSGSGTDFTLSINSVESEDI |
| | SKSQVFFKMNSLQSNDTAIYYCAR | ADYYCQQNNNWPTTFGAGTKLELK |
| | ALTYYDYEFAYWGQGTLVTVSAA | (SEQ ID NO: 7) |
| | (SEQ ID NO: 5) | GATATTCTTCTTACTCAATCTCCTGT |
| | CAAGTTCAACTTAAACAATCTGGT | TATTCTTTCTGTTTCTCCTGGTGAAC |
| | CCTGGTCTTGTTCAACCTTCTCAA | GTGTTTCTTTTTCTTGTCGTGCTTCT |
| | TCTCTTTCTATTACTTGTACTGTTT | CAATCTATTGGTACTAATATTCATT |
| | CTGGTTTTTCTCTTACTAATTATGG | GGTATCAACAACGTACTAATGGTTC |
| | TGTTCATTGGGTTCGTCAATCTCC | TCCTCGTCTTCTTATTAAATATGCTT |
| | TGGTAAAGGTCTTGAATGGCTTGG | CTGAATCTATTTCTGGTATTCCTTCT |
| | TGTTATTTGGTCTGGTGGTAATAC | CGTTTTTCTGGTTCTGGTTCTGGTAC |
| | TGATTATAATACTCCTTTTACTTCT | TGATTTTACTCTTTCTATTAATTCTG |
| | CGTCTTTCTATTAATAAAGATAAT | TTGAATCTGAAGATATTGCTGATTA |
| | TCTAAATCTCAAGTTTTTTTTAAA | TTATTGTCAACAAAATAATAATTGG |
| | ATGAATTCTCTTCAATCTAATGAT | CCTACTACTTTTGGTGCTGGTACTA |
| | ACTGCTATTTATTATTGTGCTCGT | AACTTGAACTTAAA (SEQ ID NO: 8) |
| | GCTCTTACTTATTATGATTATGAA | |
| | TTTGCTTATTGGGGTCAAGGTACT | |
| | CTTGTTACTGTTTCTGCTGCT (SEQ | |
| | ID NO: 6) | |
| EGFR | QVQLQQSGAEVKKPGSSVKVSCKA | DIQMTQSPSSLSASVGDRVTITCRSSQ |
| | SGYTFTNYYIYWVRQAPGQGLEWI | NIVHSNGNTYLDWYQQTPGKAPKLL |
| | GGINPTSGGSNFNEKFKTRVTITAD | IYKVSNRFSGVPSRFSGSGSGTDFTFT |
| | ESSTTAYMELSSLRSEDTAFYFCTR | ISSLQPEDIATYYCFQYSHVPWTFGQ |
| | QGLWFDSDGRGFDFWGQGTTVTV | GTKLQITR (SEQ ID NO: 11) |
| | SS (SEQ ID NO: 9) | GATATTCAAATGACTCAATCTCCTT |
| | CAAGTTCAACTTCAACAATCTGGT | CTTCTCTTTCTGCTTCTGTTGGTGAT |
| | GCTGAAGTTAAAAAACCTGGTTCT | CGTGTTACTATTACTTGTCGTTCTTC |
| | TCTGTTAAAGTTTCTTGTAAAGCT | TCAAAATATTGTTCATTCTAATGGT |
| | TCTGGTTATACTTTTACTAATTATT | AATACTTATCTTGATTGGTATCAAC |
| | ATATTTATTGGGTTCGTCAAGCTC | AAACTCCTGGTAAAGCTCCTAAACT |
| | CTGGTCAAGGTCTTGAATGGATTG | TCTTATTTATAAAGTTTCTAATCGTT |
| | GTGGTATTAATCCTACTTCTGGTG | TTTCTGGTGTTCCTTCTCGTTTTTCT |
| | GTTCTAATTTTAATGAAAAATTTA | GGTTCTGGTTCTGGTACTGATTTTA |
| | AAACTCGTGTTACTATTACTGCTG | CTTTTACTATTTCTTCTCTTCAACCT |
| | ATGAATCTTCTACTACTGCTTATA | GAAGATATTGCTACTTATTATTGTT |
| | TGGAACTTTCTTCTCTTCGTTCTGA | TTCAATATTCTCATGTTCCTTGGACT |
| | AGATACTGCTTTTTATTTTTGTACT | TTTGGTCAAGGTACTAAACTTCAAA |
| | CGTCAAGGTCTTTGGTTTGATTCT | TTACTCGT (SEQ ID NO: 12) |
| | GATGGTCGTGGTTTTGATTTTTGG | |
| | GGTCAAGGTACTACTGTTACTGTT | |
| | TCTTCT (SEQ ID NO: 10) | |
| IGF1R | EVQLLESGGGLVQPGGSLRLSCTAS | DIQMTQFPSSLSASVGDRVTITCRASQ |
| | GFTFSSYAMNWVRQAPGKGLEWV | GIRNDLGWYQQKPGKAPKRLIYAAS |
| | SAISGSGGTTFYADSVKGRFTISRDN | RLHRGVPSRFSGSGSGTEFTLTISSLQ |
| | SRTTLYLQMNSLRAEDTAVYYCAK | PEDFATYYCLQHNSYPCSFGQGTKLE |
| | DLGWSDSYYYYYGMDVWGQGTT | IK (SEQ ID NO: 15) |
| | VTVSS (SEQ ID NO: 13) | GATATTCAAATGACTCAATTTCCTT |
| | GAAGTTCAACTTCTTGAATCTGGT | CTTCTCTTTCTGCTTCTGTTGGTGAT |
| | GGTGGTCTTGTTCAACCTGGTGGT | CGTGTTACTATTACTTGTCGTGCTTC |
| | TCTCTTCGTCTTTCTTGTACTGCTT | TCAAGGTATTCGTAATGATCTTGGT |
| | CTGGTTTTACTTTTTCTTCTTATGC | TGGTATCAACAAAAACCTGGTAAA |
| | TATGAATTGGGTTCGTCAAGCTCC | GCTCCTAAACGTCTTATTTATGCTG |
| | TGGTAAAGGTCTTGAATGGGTTTC | CTTCTCGTCTTCATCGTGGTGTTCCT |
| | TGCTATTTCTGGTTCTGGTGGTAC | TCTCGTTTTTCTGGTTCTGGTTCTGG |
| | TACTTTTTATGCTGATTCTGTTAA | TACTGAATTTACTCTTACTATTTCTT |
| | AGGTCGTTTTACTATTTCTCGTGA | CTCTTCAACCTGAAGATTTTGCTAC |
| | TAATTCTCGTACTACTCTTTATCTT | TTATTATTGTCTTCAACATAATTCTT |
| | CAAATGAATTCTCTTCGTGCTGAA | ATCCTTGTTCTTTTGGTCAAGGTAC |
| | GATACTGCTGTTTATTATTGTGCT | TAAACTTGAAATTAAA (SEQ ID NO: |
| | AAAGATCTTGGTTGGTCTGATTCT | 16) |
| | TATTATTATTATTATGGTATGGAT | |
| | GTTTGGGGTCAAGGTACTACTGTT | |
| | ACTGTTTCTTCT (SEQ ID NO: 14) | |
| IGF1R | EVQLVQSGAEVKKPGSSVKVSCKA | SSELTQDPAVSVALGQTVRITCQGDS |
| | SGGTFSSYAISWVRQAPGQGLEWM | LRSYYATWYQQKPGQAPILVIYGEN |
| | GGIIPIFGTANYAQKFQGRVTITADK | KRPSGIPDRFSGSSSGNTASLTITGAQ |
| | STSTAYMELSSLRSEDTAVYYCAR | AEDEADYYCKSRDGSGQHLVFGGGT |
| | APLRFLEWSTQDHYYYYYMDVWG | KLTVLGQPKAA (SEQ ID NO: 19) |
| | KGTTVTVSS (SEQ ID NO: 17) | TCTTCTGAACTTACTCAAGATCCTG |
| | GAAGTTCAACTTGTTCAATCTGGT | CTGTTTCTGTTGCTCTTGGTCAAACT |
| | GCTGAAGTTAAAAAACCTGGTTCT | GTTCGTATTACTTGTCAAGGTGATT |
| | TCTGTTAAAGTTTCTTGTAAAGCT | CTCTTCGTTCTTATTATGCTACTTGG |
| | TCTGGTGGTACTTTTTCTTCTTATG | TATCAACAAAAACCTGGTCAAGCTC |
| | CTATTTCTTGGGTTCGTCAAGCTC | CTATTCTTGTTATTTATGGTGAAAA |
| | CTGGTCAAGGTCTTGAATGGATGG | TAAACGTCCTTCTGGTATTCCTGAT |
| | GTGGTATTATTCCTATTTTTGGTA | CGTTTTTCTGGTTCTTCTTCTGGTAA |
| | CTGCTAATTATGCTCAAAAATTTC | TACTGCTTCTCTTACTATTACTGGTG |
| | AAGGTCGTGTTACTATTACTGCTG | CTCAAGCTGAAGATGAAGCTGATT |
| | ATAAATCTACTTCTACTGCTTATA | ATTATTGTAAATCTCGTGATGGTTC |
| | TGGAACTTTCTTCTCTTCGTTCTGA | TGGTCAACATCTTGTTTTTGGTGGT |
| | AGATACTGCTGTTTATTATTGTGC | GGTACTAAACTTACTGTTCTTGGTC |
| | TCGTGCTCCTCTTCGTTTTCTTGAA | AACCTAAAGCTGCT (SEQ ID NO: 20) |
| | TGGTCTACTCAAGATCATTATTAT | |
| | TATTATTATATGGATGTTTGGGGT | |
| | AAAGGTACTACTGTTACTGTTTCT | |
| | TCT (SEQ ID NO: 18) | |
| IGF1R | QVQLQESGPGLVKPSETLSLTCTVS | DIVMTQSPLSLPVTPGEPASISCRSSQS |
| | GYSISGGYLWNWIRQPPGKGLEWI | IVHSNGNTYLQWYLQKPGQSPQLLIY |
| | GYISYDGTNNYKPSLKDRVTISVDT | KVSNRLYGVPDRFSGSGSGTDFTLKI |
| | SKNQFSLKLSSVTAADTAVYYCAR | SRVEAEDVGVYYCFQGSHVPWTFGQ |
| | YGRVFFDYWGQGTLVTVSSA (SEQ | GTKVEIK (SEQ ID NO: 23) |
| | ID NO: 21) | GATATTGTTATGACTCAATCTCCTC |
| | CAAGTTCAACTTCAAGAATCTGGT | TTTCTCTTCCTGTTACTCCTGGTGAA |
| | CCTGGTCTTGTTAAACCTTCTGAA | CCTGCTTCTATTTCTTGTCGTTCTTC |
| | ACTCTTTCTCTTACTTGTACTGTTT | TCAATCTATTGTTCATTCTAATGGT |
| | CTGGTTATTCTATTTCTGGTGGTT | AATACTTATCTTCAATGGTATCTTC |
| | ATCTTTGGAATTGGATTCGTCAAC | AAAAACCTGGTCAATCTCCTCAACT |
| | CTCCTGGTAAAGGTCTTGAATGGA | TCTTATTTATAAAGTTTCTAATCGTC |
| | TTGGTTATATTTCTTATGATGGTA | TTTATGGTGTTCCTGATCGTTTTTCT |
| | CTAATAATTATAAACCTTCTCTTA | GGTTCTGGTTCTGGTACTGATTTTA |
| | AAGATCGTGTTACTATTTCTGTTG | CTCTTAAAATTTCTCGTGTTGAAGC |
| | ATACTTCTAAAAATCAATTTTCTC | TGAAGATGTTGGTGTTTATTATTGT |
| | TTAAACTTTCTTCTGTTACTGCTGC | TTTCAAGGTTCTCATGTTCCTTGGA |
| | TGATACTGCTGTTTATTATTGTGC | CTTTTGGTCAAGGTACTAAAGTTGA |
| | TCGTTATGGTCGTGTTTTTTTTGAT | AATTAAA (SEQ ID NO: 24) |
| | TATTGGGGTCAAGGTACTCTTGTT | |
| | ACTGTTTCTTCTGCT (SEQ ID NO: | |
| | 22) | |

### Example 2

### Size-Exclusion Chromatography (SEC-HPLC) of Protein a Purified iMab-EI

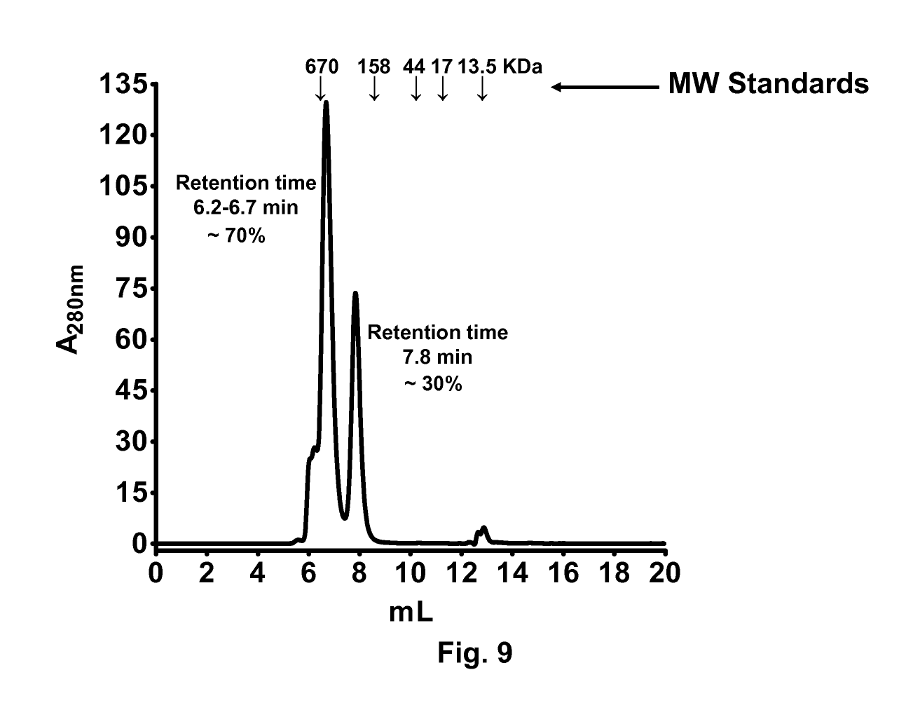

The iMab-EI after protein A purification was analyzed by analytical size-exclusion chromatography (FIG. 9). Size-exclusion HPLC chromatography (SEC-HPLC, Agilent 1100 Capillary LC System) was used to determine the molecular weights (MW, Dalton) and the monomeric content of constructs. The wavelength was set to 280 nm and the experiments were carried out at 25° C. SEC-HPLC was carried out using a TSK-GEL G3000SWXL column (Tosoh Bioscience LLC, Montgomeryville, Pa.), which separates globular proteins with MW that range from approximately 10 to 500 kDa, with a buffer containing 100 mM sodium phosphate, pH 6.8, and at a flow rate of 1 ml/min. A low molecular weight gel filtration calibration kit from Bio-Rad (Hercules, Calif.) containing vitamin B12 (11,350 Da), equine myoglobin (17,000 Da), chicken ovalbumin (44,000 Da), bovine gamma-globulin (158,000 Da) and thyroglobulin (670,000 Da) was used as molecular mass standards. In addition, a highly purified 99% monomeric IgG was used as immunoglobulin molecular weight standard. The SEC-HPLC analysis (FIG. 9), of the purified iMab-EI, shows that the iMab-EI exists as conformational aggregate with two major peaks: one with a retention time between 6.2 and 6.7 minutes and one with a retention time of 7.8 minutes.

### Example 3

### ELISA Assays for Determining Functional Binding of the iMab-EI Having Intact Linkers to its Respective Antigens

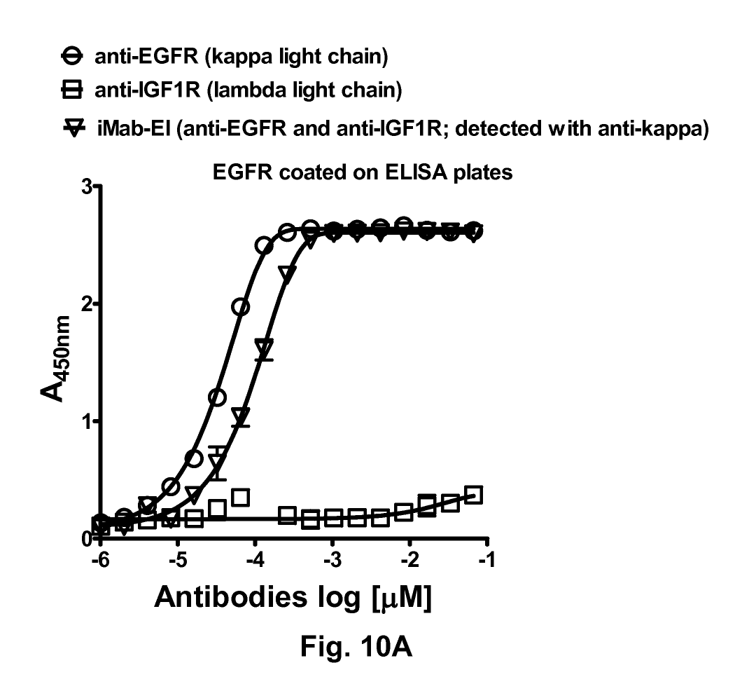

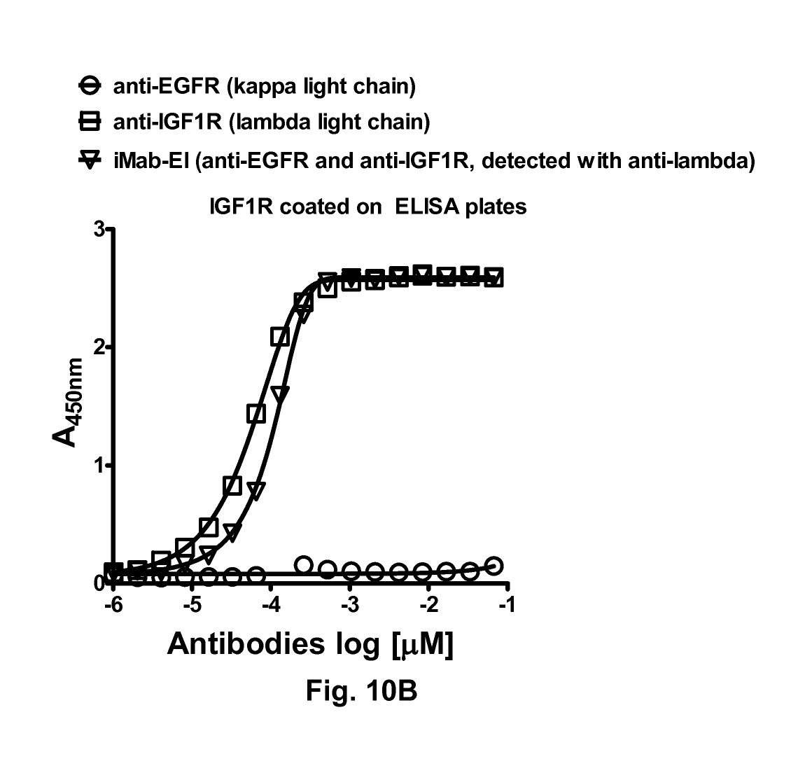

As shown in FIG. 10, the intact iMab-EI is able to bind its two antigens, in this specific example EGFR and IGF1R. For the ELISA binding assay, 2 μg/mL of antigen in 30 μL of PBS, pH 7.4 was coated on microtiter wells for 1 hour at room temperature. Antigen-coated wells were washed 3 times with PBS containing 0.1% (v/v) Tween-20 and blocked for one hour at room temperature with 3% BSA. Antibodies were serially diluted in 30 μL of blocking solution and were incubated for 2 hour at 37° C., followed by extensive washes with PBS containing 0.1% (v/v) Tween-20. Bound antibodies were detected by HRP-conjugated anti-kappa or anti-lambda secondary antibodies and visualized with 30 μL of 3,3′,5,5′-tetramethylbenzidine substrate (Pierce). The reaction was stopped by adding 30 μL of 0.18 M sulfuric acid (Pierce). The absorbance at 450 nm was measured using a microtiter plate reader. The resulting data were analyzed using Prism 5 software (GraphPad, San Diego, Calif.). As shown in FIG. 10, the two conventional antibodies bind specifically to their respective antigens; the anti-EGFR conventional antibody binds to EGFR but not to IGF1R, and the anti-IGF1R conventional antibody binds to IGF1R but not to EGFR. In contrast, the innovative monoclonal antibody iMab-EI is capable of binding both antigens: EGFR and IGF1R. Moreover, the specific binding of iMab-EI for both antigens can be detected using secondary antibodies specific for anti-kappa or anti-lambda.

### Example 4

### Dual ELISA Assays for Determining Functional Concurrent Binding of the iMab-EI with Intact Linkers to its Respective Antigens

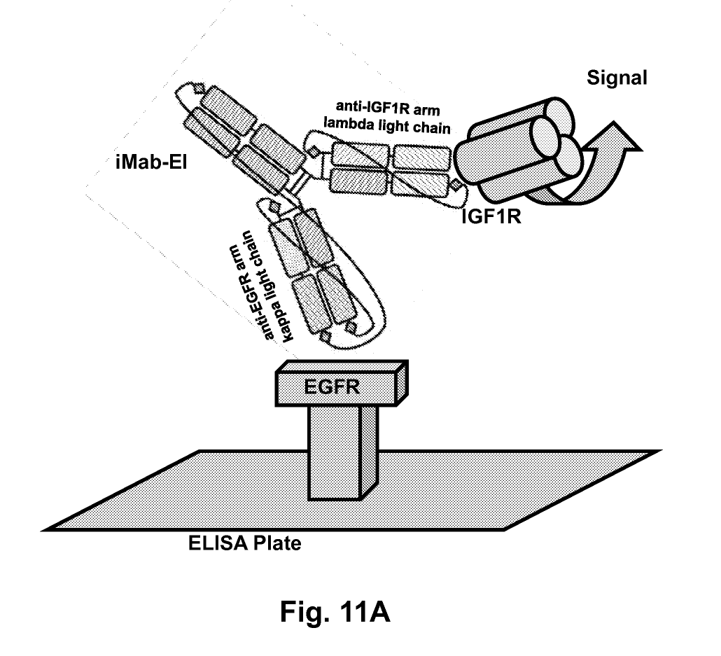

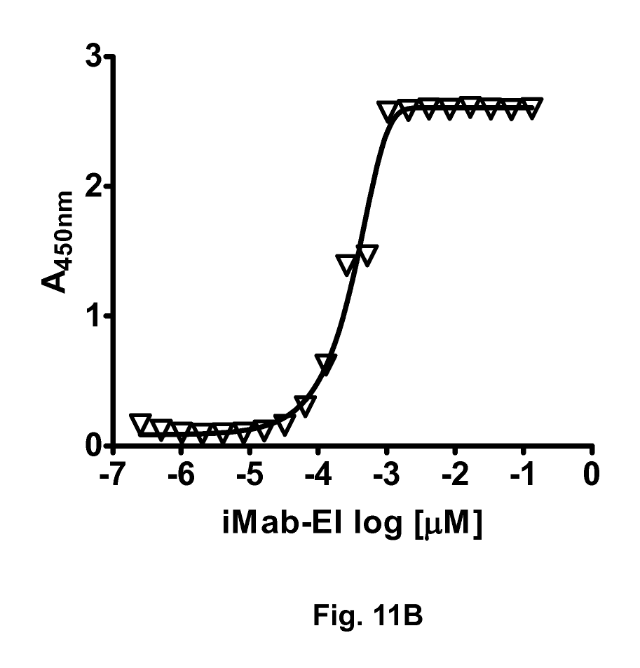

As shown in FIG. 11, the intact iMab-EI is capable of concurrently binding its two antigens. Dual ELISA binding was carried out by direct immobilization on the ELISA plate of the first antigen (EGFR) at the same concentration and conditions as described in example 3. After blocking, iMab-EI with intact linkers in serial dilutions was added and incubated for 1 hour at room temperature; followed by the addition of the second histidine-tagged antigen (IGF1R) at 2 μg/ml. Detection was carried out using an HRP-conjugated anti-histidine antibody (Qiagen). Color development was allowed to proceed for approximately 5 minutes. The reaction was quenched by the addition of 0.5 M H2SO4, 100 μL per well, and absorbance at 450 nm was read.

### Example 5

### Removal of the iMab-EI Linkers with Thrombin and Analysis Using SDS-PAGE and SEC-HPLC

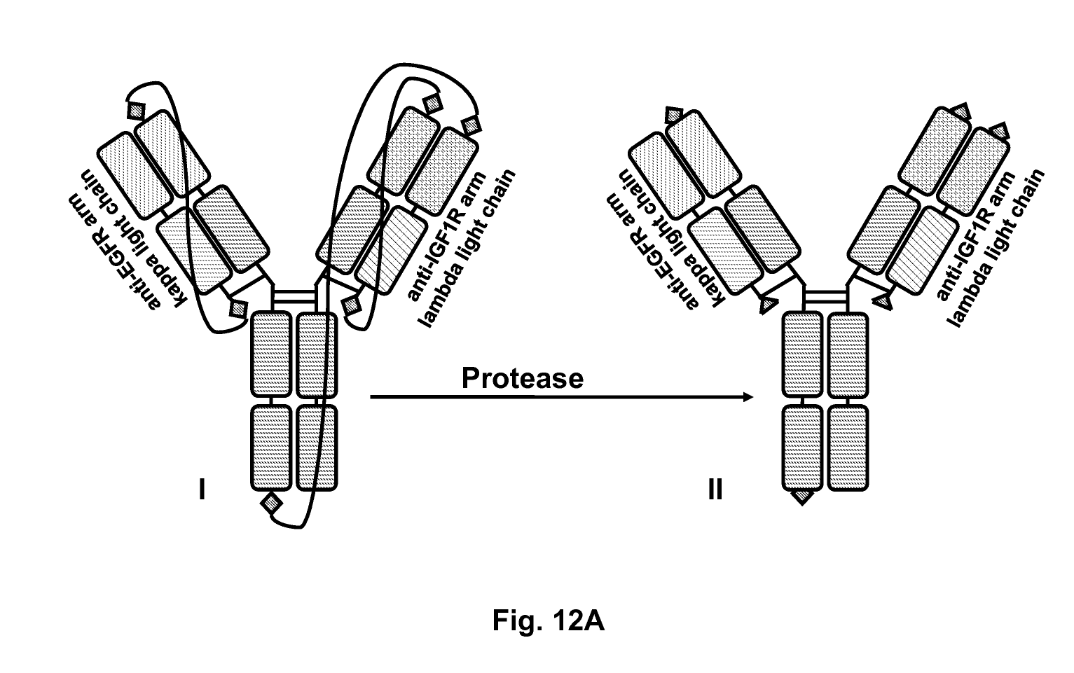

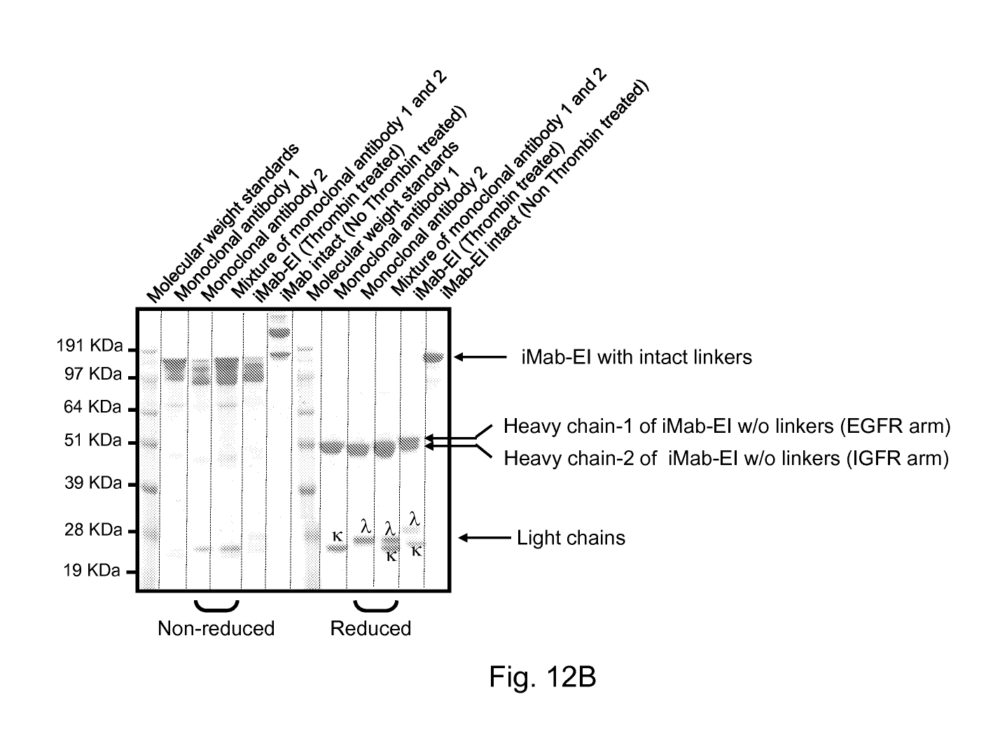

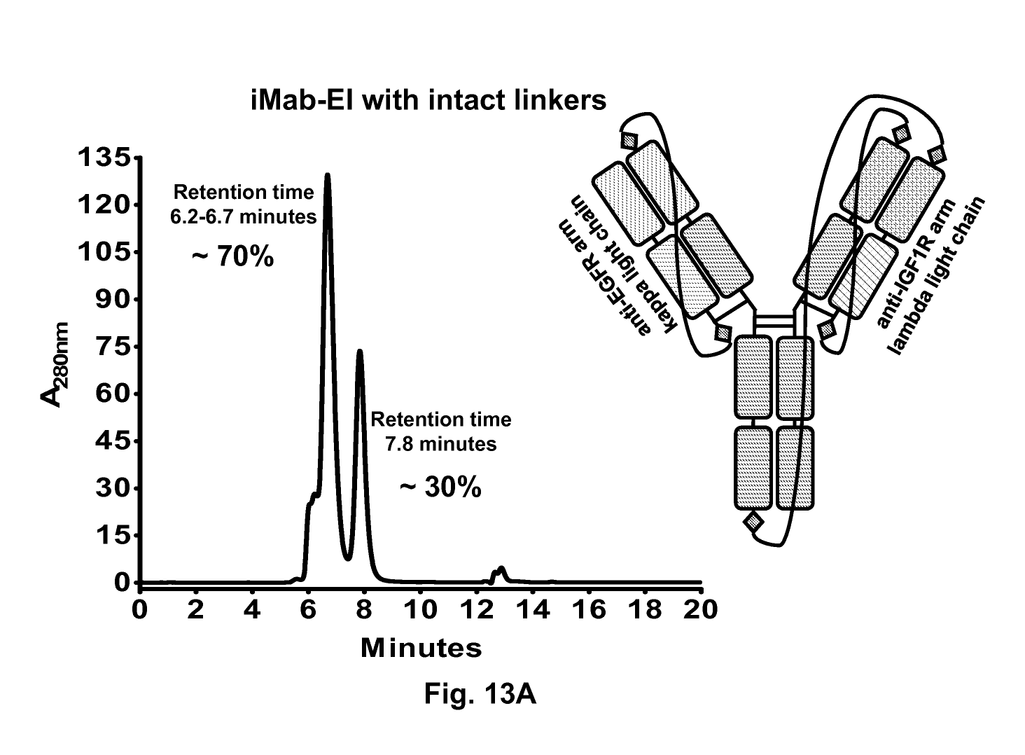

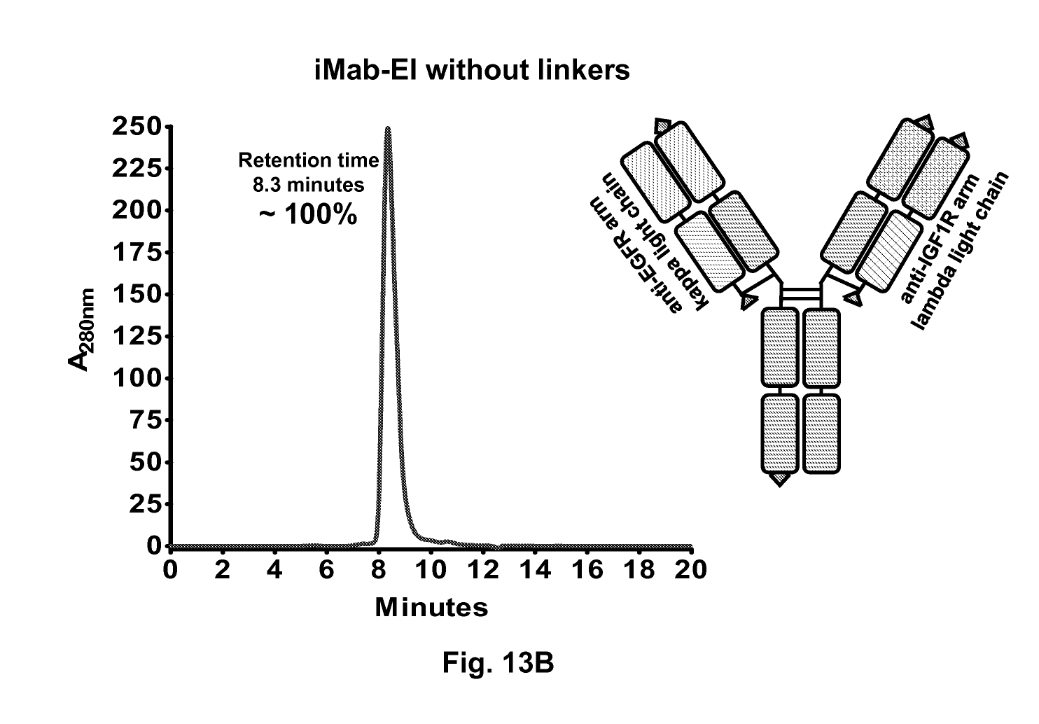

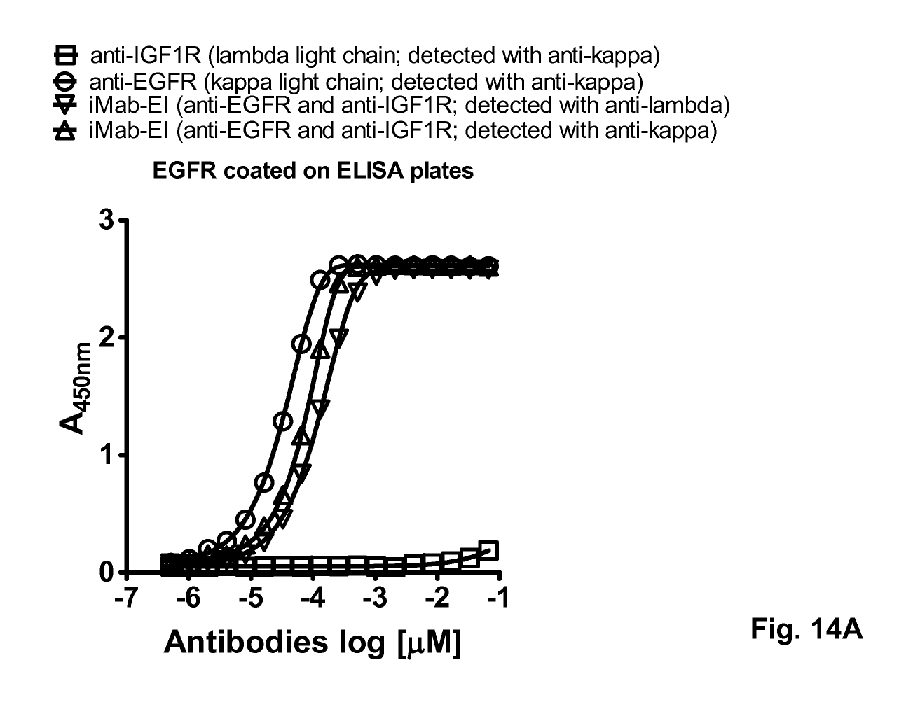

Removal of the linkers is schematically depicted in FIG. 12A. The protein A purified iMab-EI with linker was incubated with 10 Units of Thrombin (Sigma) in PBS 1×pH 7.2 for 0.5 mg total iMab-EI. The incubation was carried out at 37° C. or 4° C. with near 100% efficiency for a minimum time of 30 minutes to a maximum time of overnight. The protease digestion was analyzed using SDS-PAGE analysis (FIG. 12B). The SDS-PAGE experimental condition used is as described in Example 1. As shown in FIG. 12B, the intact iMab-EI, without protease treatment and under reducing conditions, runs as a distinct single band, whereas treatment with the protease releases the individual components of the iMab-EI (namely, the two heavy chains, heavy chain 1 anti-EGFR arm; and heavy chain 2 anti-IGF1R arm; and the two light chain, the anti-EGFR kappa light chain; and the anti-IGF1R lambda light chain). In addition to the SDS-PAGE analysis, the linker removal was also analyzed by SEC-HPLC as shown in FIG. 13. SEC-HPLC was carried out essentially as described in example 2. As shown in FIG. 13, prior to protease treatment the iMab-EI has two major conformational peaks (FIG. 13A, and FIG. 9) however, after protease treatment the iMab-EI has a near 100% monomeric and homogeneous peak that runs with a retention time of 8.3 minutes (FIG. 13 B).

### Example 6

### ELISA Assays for Determining Functional Binding of the iMab-EI, Following Removal of Linkers, to its Respective Antigens

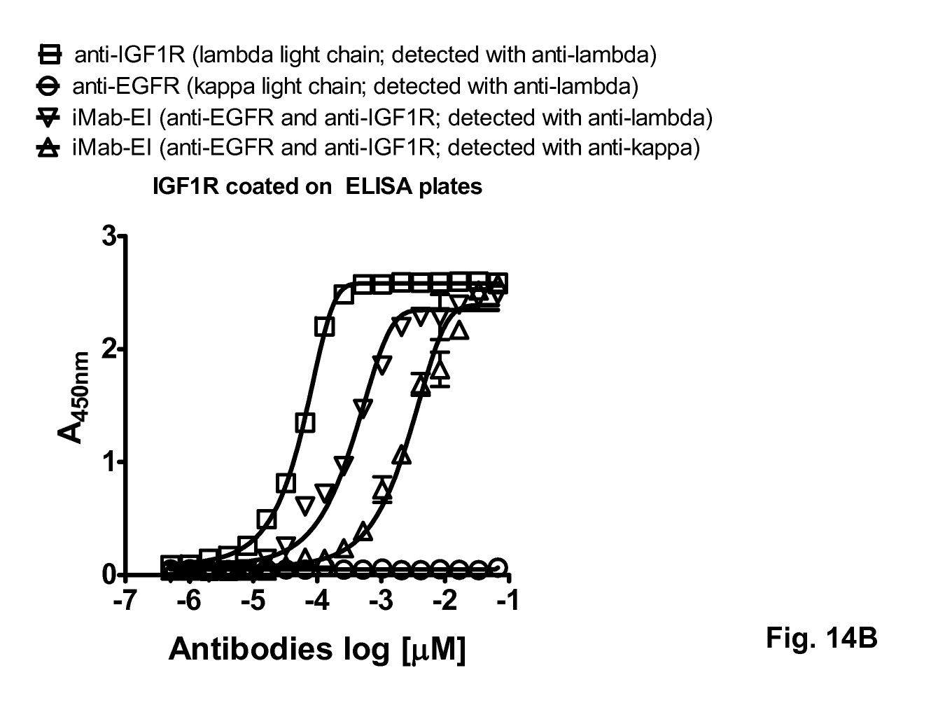

As shown in FIG. 14, the iMab-EI with removed linkers is able to bind its two antigens, in this specific example EGFR (FIG. 14A) and IGF1R (FIG. 14B). The ELISA was carried out as described in example 3. As shown in FIG. 14, the two conventional antibodies bind specifically to their respective antigens; the anti-EGFR conventional antibody binds to EGFR but not to IGF1R, and the anti-IGF1R conventional antibody binds to IGF1R but not to EGFR. In contrast, the innovative monoclonal antibody iMab-EI, following removal of the linkers, is capable of binding both antigens: EGFR and IGF1R. Moreover, the specific binding of iMab-EI for its two antigens can be detected using secondary antibodies specific for anti-kappa or anti-lambda.

### Example 7

### Dual ELISA Assays for Determining Functional Concurrent Binding of the iMab-EI with Removed Linkers to its Respective Antigens

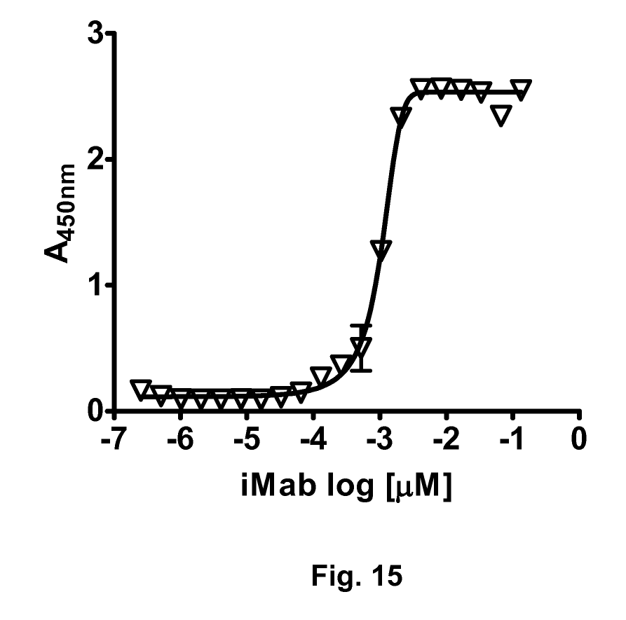

As shown in FIG. 15, the iMab-EI, with removed linkers, is capable of concurrent binding to its antigens: EGFR and IGF1R. Dual ELISA binding was carried out as described in example 4.

### Example 8

### iMab-EI has as a Unique Retention Time when Analyzed by SEC-HPLC that does Not Correspond to the Retention Times of the Two Conventional Antibodies

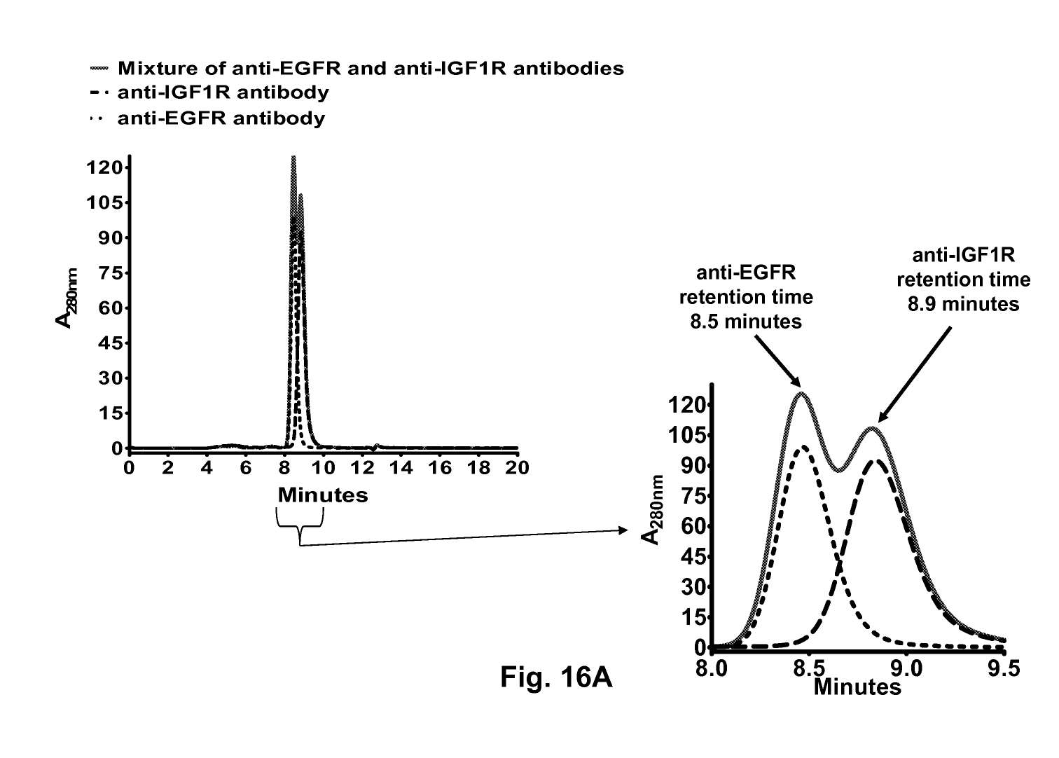

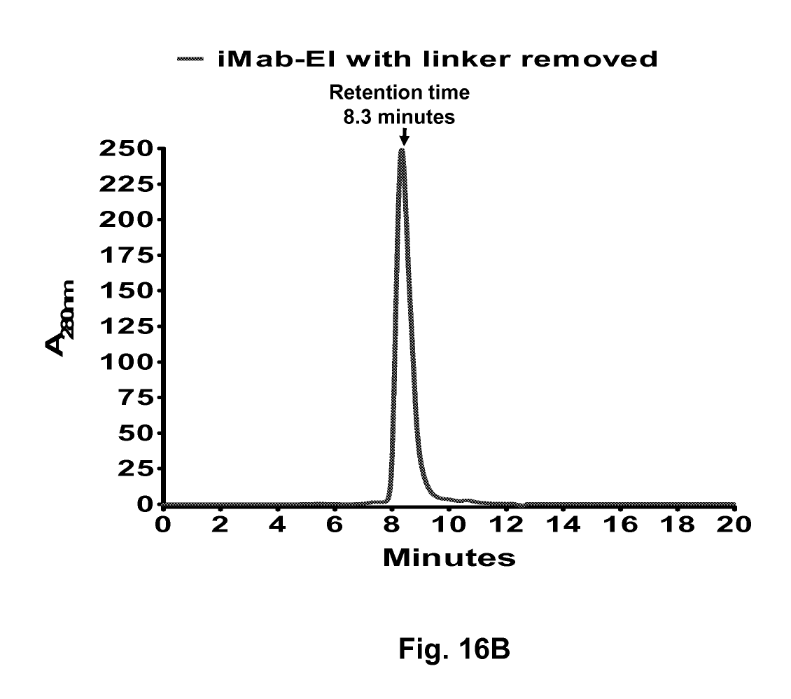

FIG. 16 show the SEC-HPLC analysis carried out as described in example 2. As shown in FIG. 16, iMab-EI retention time under the experimental condition used is 8.3 minutes from the injection; whereas the retention time for the two conventional antibodies is 8.9 minutes for the anti-IGF1R antibody and 8.5 minutes for the anti-EGFR antibody.

### Example 9

### Isoelectric Point (pI) Determination

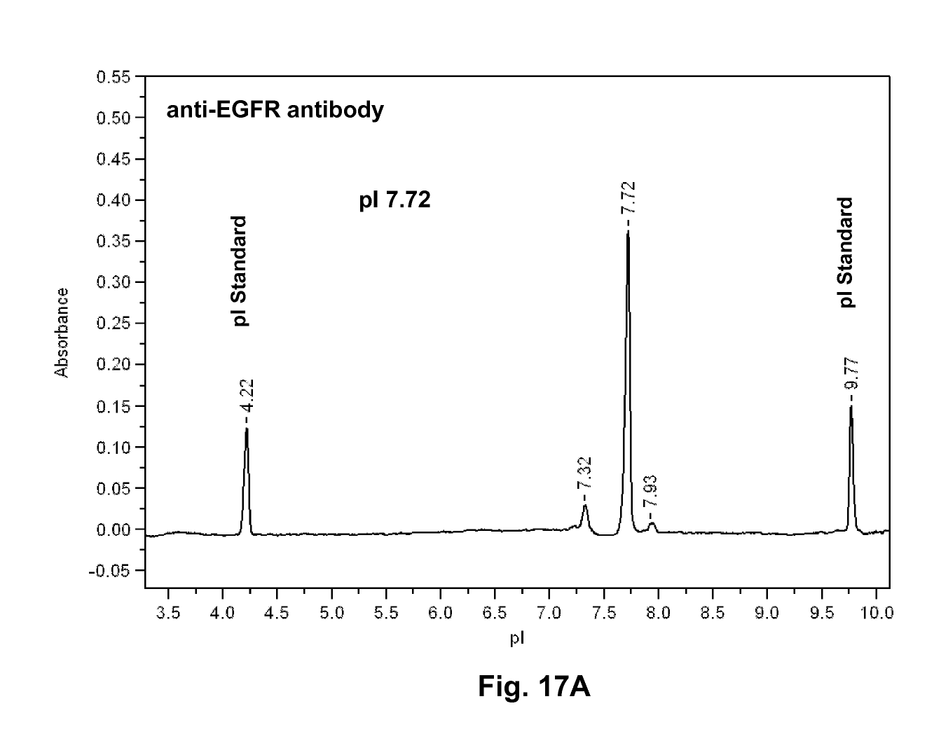

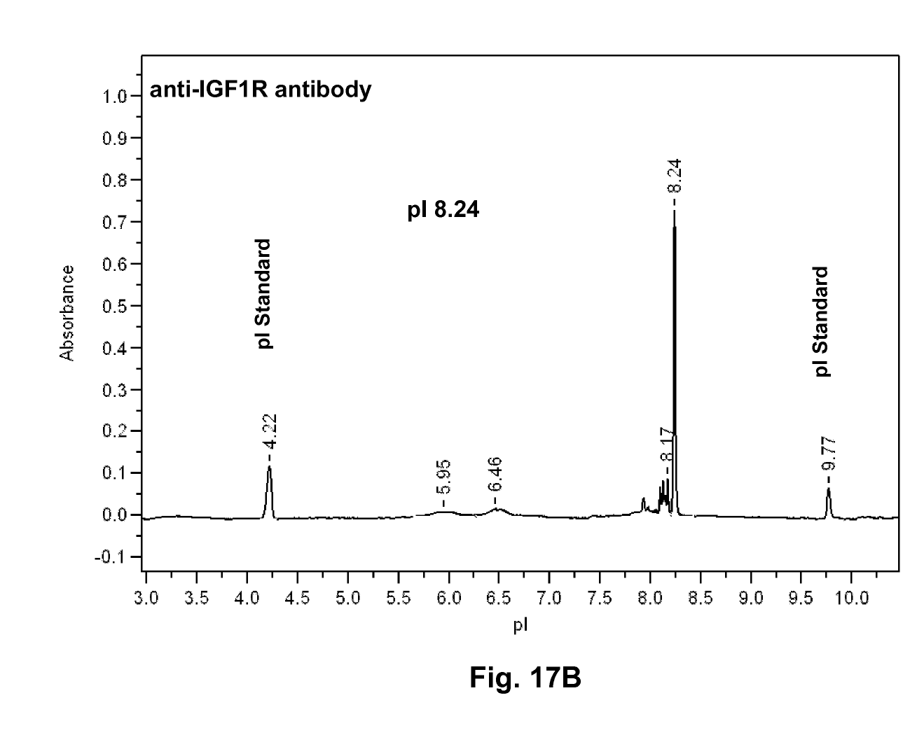

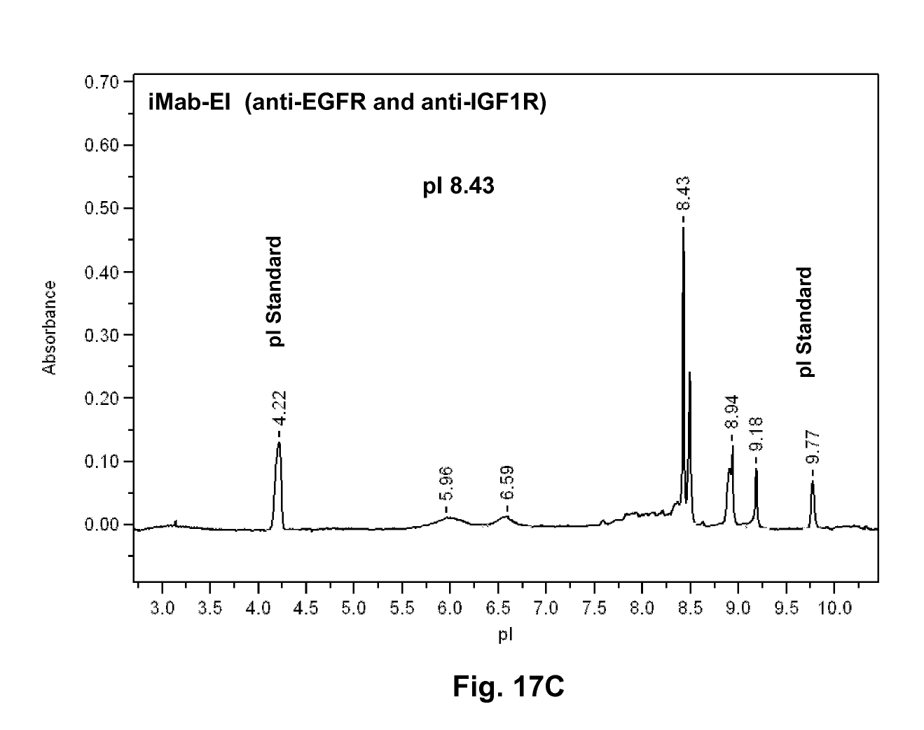

pI was determined using 250 μg total of recombinant product in a volume of 200 μL. iCE280 from Convergent Bioscience (Toronto, Canada) was used to determine the pI of the iMab-EI and the two conventional antibodies. pI determination was carried out essentially as described by the manufacturer. Briefly, iCE280 performs free solution IEF in a capillary column (cIEF) and detects focused protein zones using a whole column UV absorption detector. The separation column of the analyzer was a 5 cm long, 100 μm i.d. silica capillary (Polymicro Technologies, Tucon, Ariz., USA) coated with fluorocarbon. All separations were performed under 5 kV focusing voltage (600 V/cm). All chemicals used in the experiments were of reagent grade and prepared in deionized water of an HPLC reagent. Phosphoric acid (87%, reagent grade) and sodium hydroxide (50% w/w, reagent grade) were purchased from J. T. Baker (Phillipsburg, N.J., USA). Pharmalyte pH 3-10, Pharmalyte pH 2.5-5, Pharmalyte pH 8-10.5, Ampholyte pH 4-7, and PBS buffer were purchased from Sigma (St. Louis, Mo., USA). pI markers were purchased from BioRad Laboratories (Mississauga, ON, Canada). Methylcellulose (MC, 1%) was obtained from Convergent Bioscience (Toronto, Canada). pI 4.40, and 9.61 were used as pI markers and were at 2 mg/mL solutions. Final sample solutions were made by mixing the samples with carrier ampholytes, 1% MC solution and water. In all the experiments, the concentration of carrier ampholytes in the final sample solutions was 4% (4 mL carrier ampholytes in 100 mL final solutions), and the concentration of MC was 0.35%. These pI experiments are shown in FIG. 17, and confirm that the iMab-EI has a unique pI of 8.43, which is different from the pI of the anti-EGFR and anti-IGF1R conventional antibodies (pI values of 7.72 and 8.24, respectively).

### Example 10

### Differential Scanning Calorimetry (DSC) Analysis of the Anti-EGFR and Anti-IGF1R Conventional Antibodies and the iMab-EI

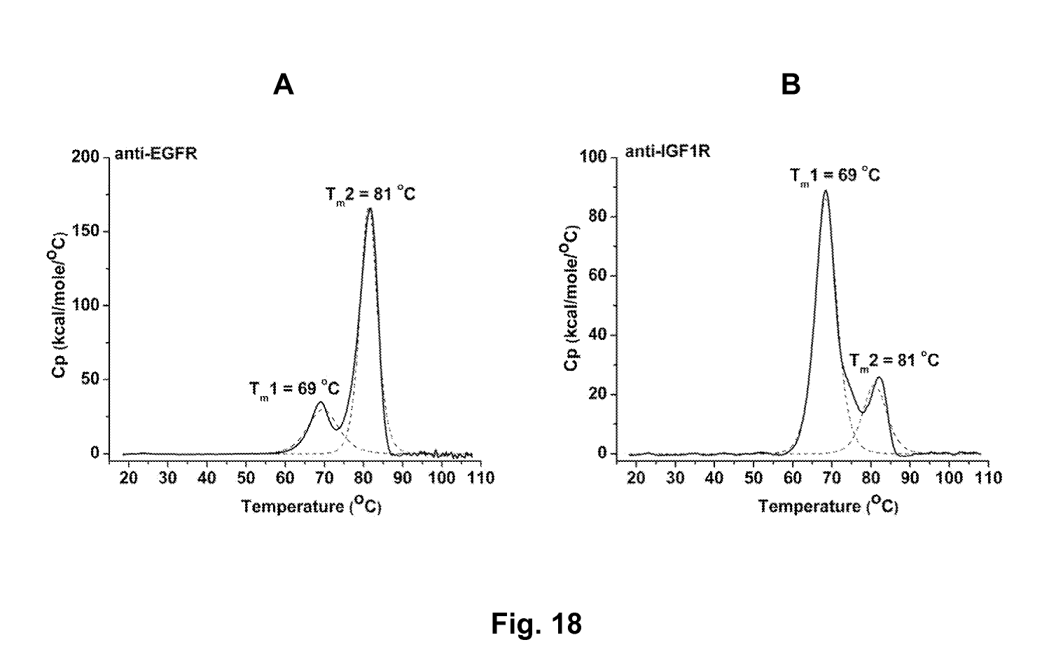

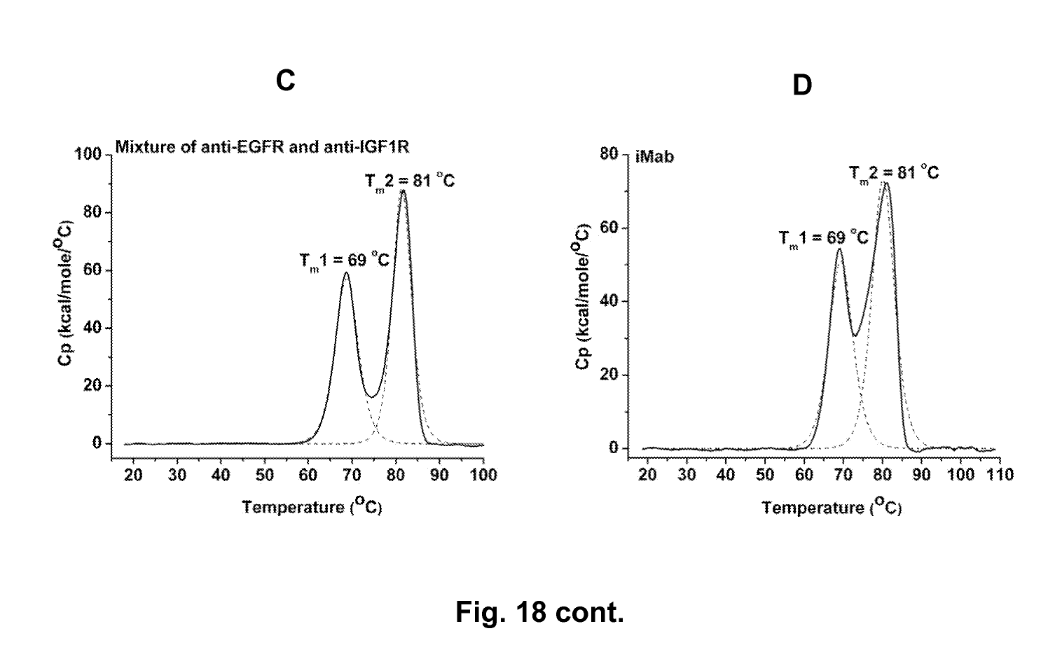

Differential scanning calorimetry (DSC) experiments, as depicted in FIG. 18, at a heating rate of 1° C./min were carried out using a Microcal VP-DSC ultrasensitive scanning microcalorimeter (Microcal, Northampton, Mass.). DSC experiments were carried out in 25 mM Histidine-HCl, pH 6. All solutions and samples used for DSC were filtered using a 0.22 micron-filter and degassed prior to loading into the calorimeter. The two conventional antibodies, their mixture and the iMab-EI that were used for the DSC studies were >99% monomeric as judged by analytical gel filtration chromatography. For each set of measurements, at least four buffer-versus-baseline runs were first obtained. Immediately after, the buffer solution was removed from the sample cell and loaded with approximately 0.75 ml of sample at concentration of 1 mg/ml. For each measurement the reference cell was filled with the sample buffer. In each sample-versus-buffer experiment, the corresponding buffer-versus-buffer baseline run was subtracted. The raw data were normalized for concentration and scan rate. Data analysis and deconvolution was carried out using the Origin™ DSC software provided by Microcal.

### Example 11

### ELISA Depletion Analysis of the Anti-EGFR and Anti-IGF1R Conventional Antibodies and the iMab-EI

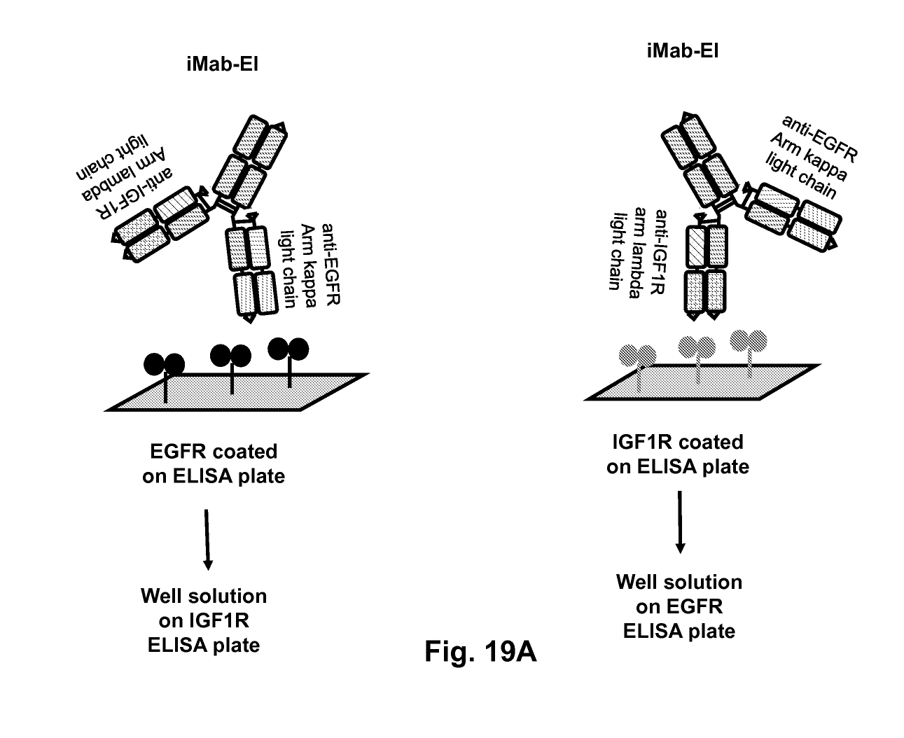

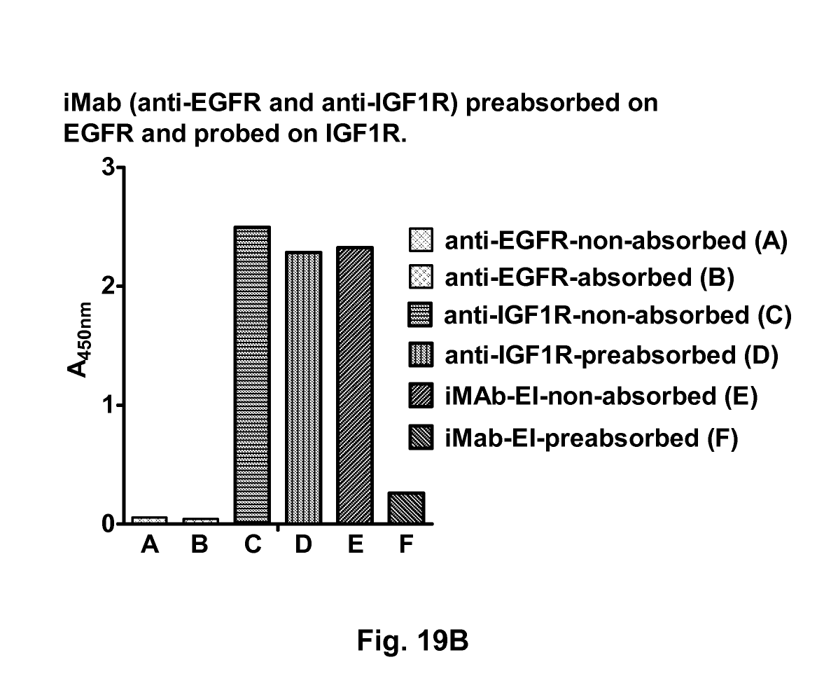

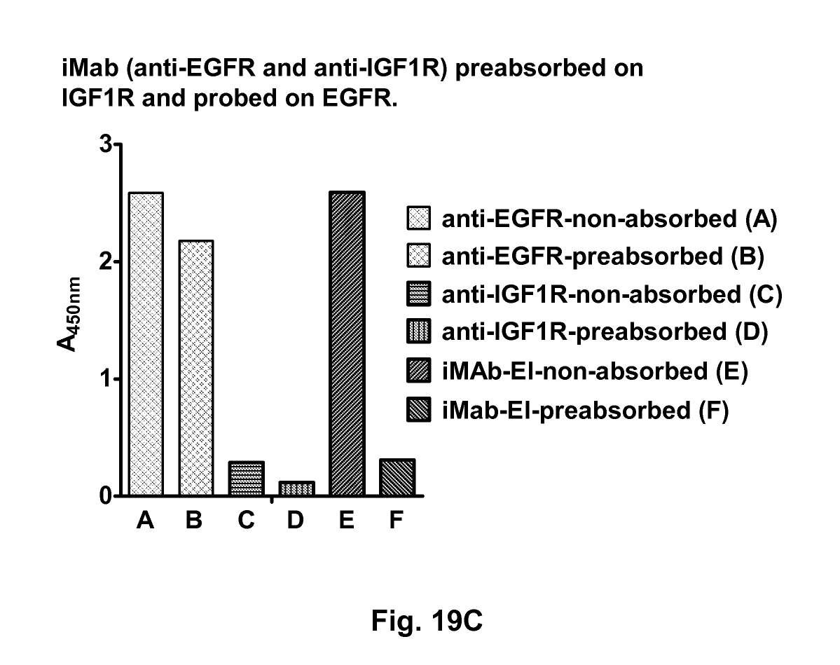

ELISA experiments, as depicted in FIG. 19, were carried out to verify that the iMab-EI is a homogeneous preparation and does not contain either one of the two parental antibody arms. The depletion ELISA assay consisted of pre-incubating the iMab-EI (0.02 μg/mL) or the conventional parental control antibodies (0.02 μg/mL) on one antigen and then probing the well supernatant against the other antigen. If the well supernatant of the preabsorbed iMab-EI shows a minimal signal this indicates that the iMab-EI preparation is homogeneous (i.e., only iMab-EI is present). The procedure for the iMab-EI ELISA absorption on EGFR is as follows. ELISA plates were coated overnight with 2 μg/mL (30 mL/well) of EGFR-FC (identified as depletion plates) and with 2 μg/mL (30 mL/well) of IGF1R (identified as probe plate) in PBS. Both ELISA plates were washed 5 times with PBST and blocked with 3% NFDM in PBST for 1 hour at RT. A solution of 0.02 μg/mL of anti-EGFR, anti-IGF1R and iMab-EI was added to the EGFR-FC plates (30 μL/well) and incubated at RT for 1 hour. The well solutions (30 μL/well) from the EGFR plates were then transferred to the IGF1R wells with subsequent incubation for one hour at room temperature. The ELISA probe plates were washed 5 times with PBST and a 1:3000 dilution of goat anti-human-kappa-HRP/goat anti-human-lambda-HRP secondary antibodies mixture was added. The plates were washed 5 times with PBST and developed with TMB. A similar procedure was used for the iMab-EI ELISA absorbed on IGF1R, but probed on EGFR.

### Example 12

### In Vivo Efficacy of iMab-EI Using Human Primary Tumor Xenograft

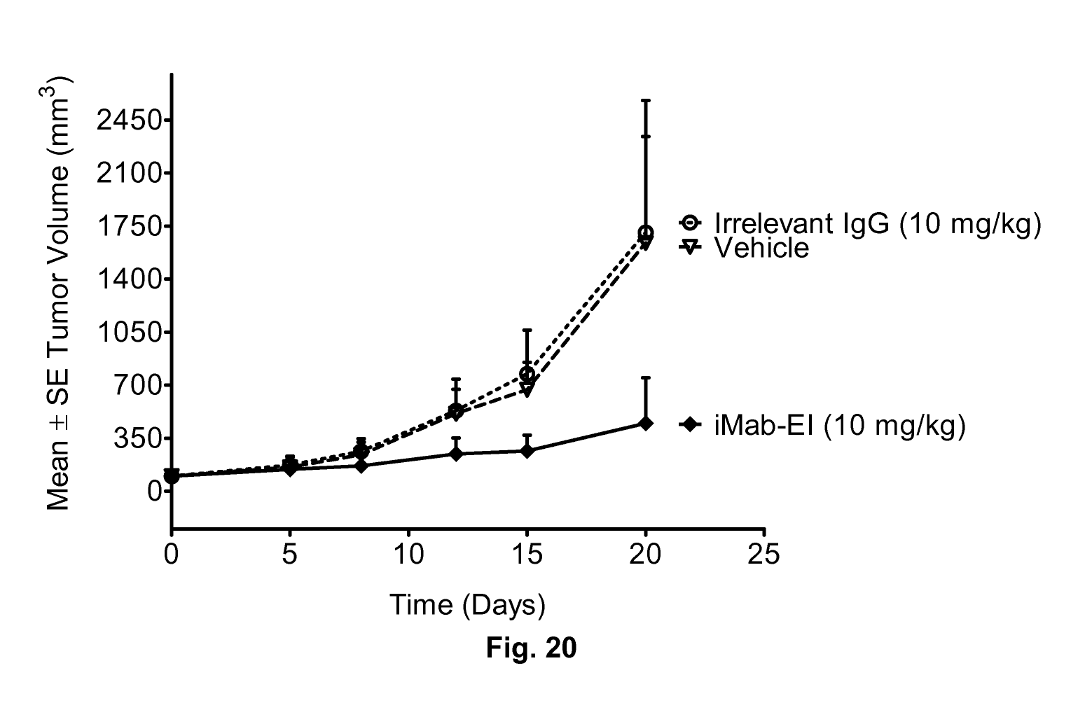

The example shown in FIG. 20 is tumor growth curves of primary human xenograft tumor in Rag2ko mice. In this example all mice groups (iMab-EI, vehicle control, and irrelevant isotype antibody control) were dosed at 10 mg/kg. 8 female Rag2ko mice were used in each group. The reported points (obtained at day 5, 8, 12, 15, and 20 post-inoculation) are average of tumor volume versus time of start of treatment. Mice were dosed with the antibodies when tumor reached an average volume size of 90 millimeters cubed. The tumor growth inhibition curves indicate that iMab-EI is efficacious in vivo. The animal experiments described herein were approved by the MedImmune Administrative Panel for Laboratory Animal Care.

### Example 13

### Alternative Protease Cleavage of iMers

It is contemplated that a wide variety of protease cleavage sites could be engineered into an iMer. As described above, a thrombin cleavage site (e.g., LVPR↓GS (SEQ ID NO: 48), arrow (↓) indicates the site of cleavage) may be incorporated into one or more of the linkers. Additional non-limiting examples of cleavage sites that may be used are provided below.

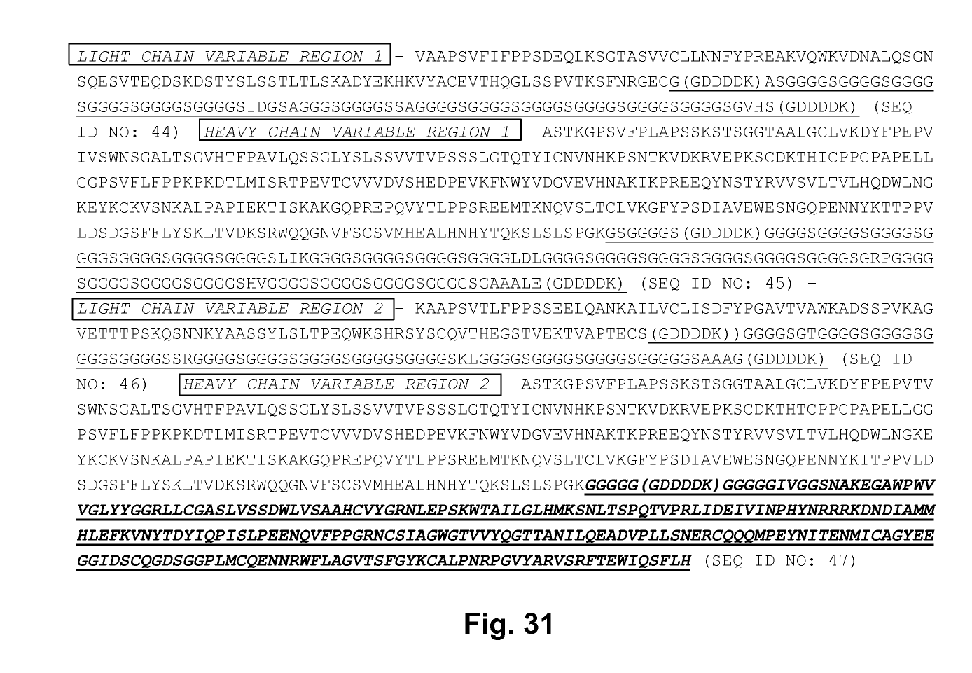

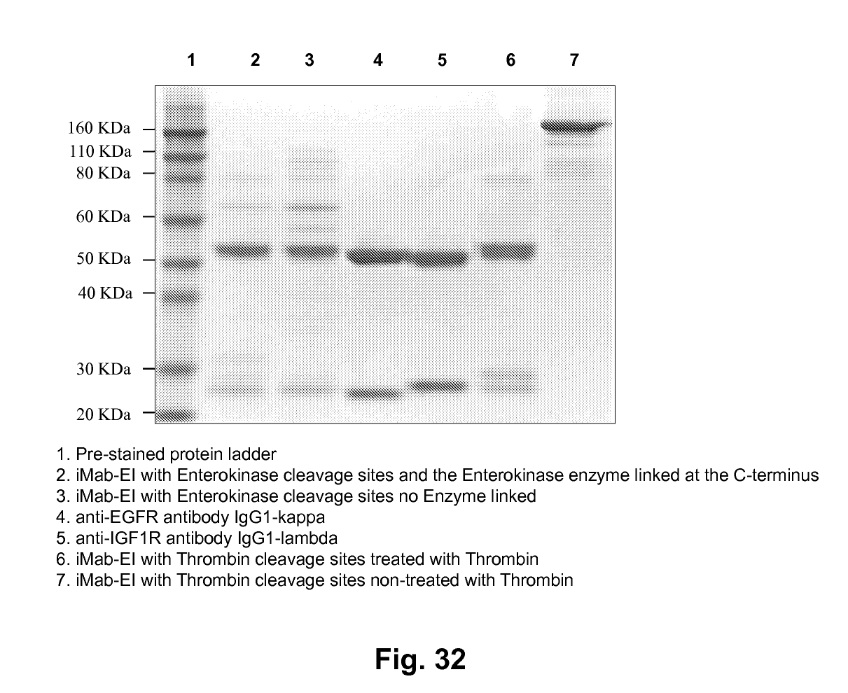

FIG. 21 provides a schematic representation of an iMer construct, an iMab in this example, in which a protease is linked to a protease that can undergo auto-cleavage. The linked protease may be encoded as part of the contiguous polypeptide chain. FIG. 31 provides the amino acid sequence of a representative iMer, an iMab in this example, with intact linkers having Enterokinase cleavage sites identified by the sequence GDDDK (SEQ ID NO: 25), and a catalytically active human Enterokinase—light chain—(shown in bold and underlined).

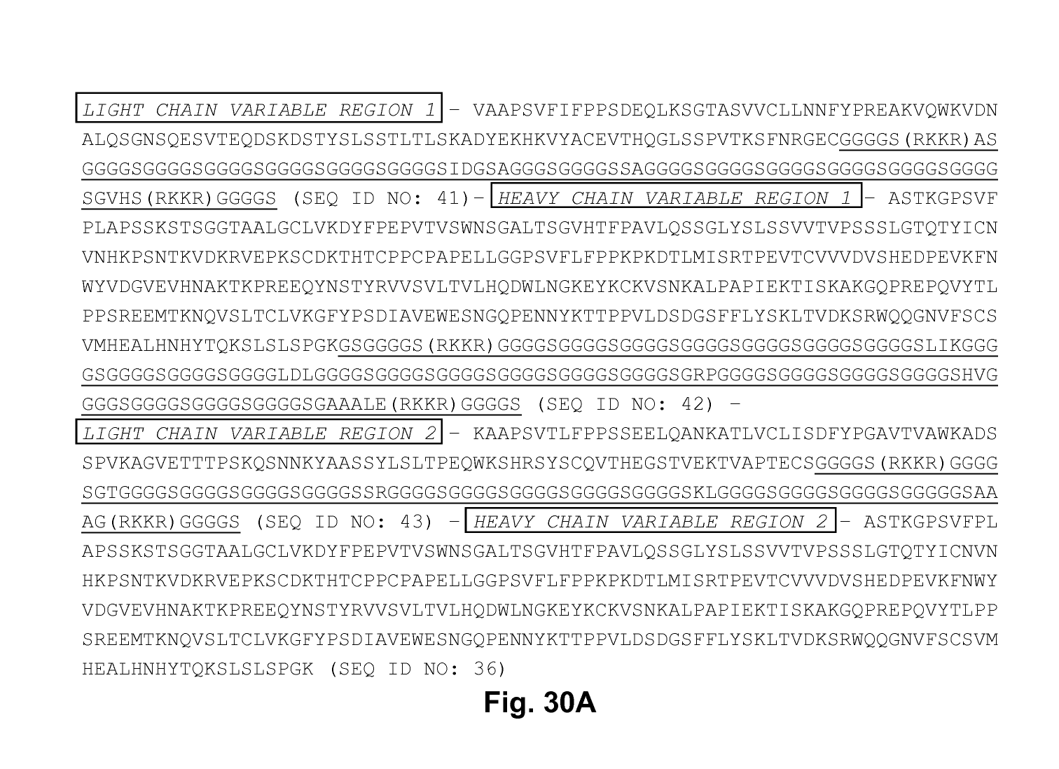

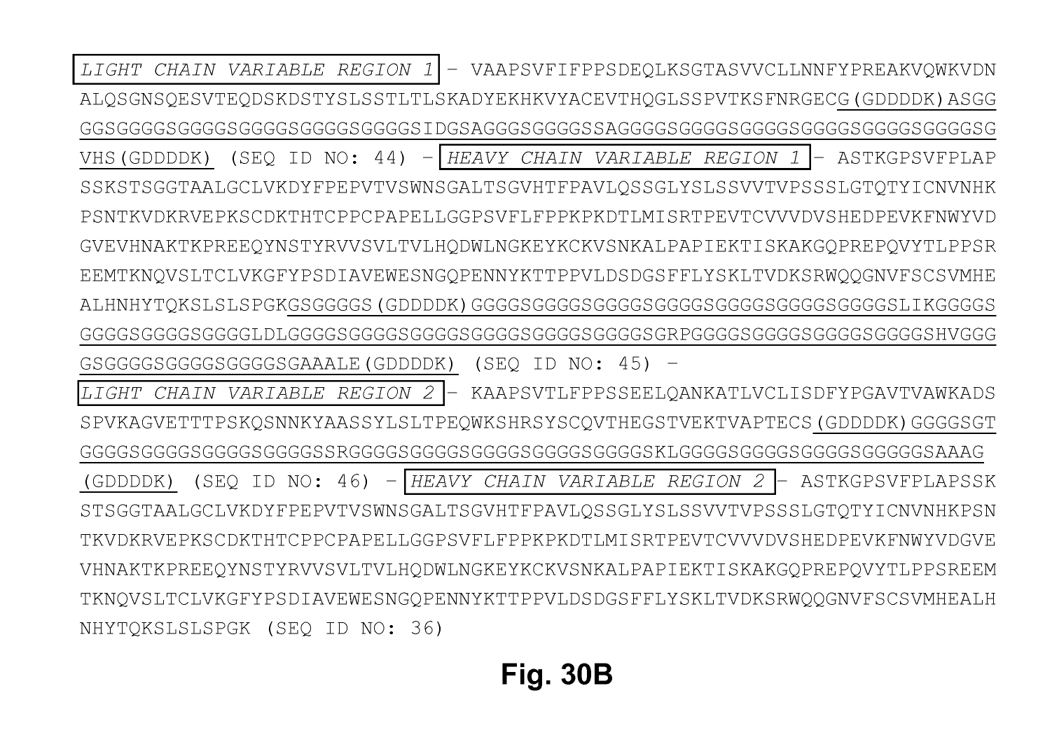

FIG. 22 provides a schematic diagram of the use of a cellular protease to cleave the iMer linkers, an iMab in this example, without the use of exogenously added protease. FIG. 30A provides the amino acid sequence of a representative iMer, an iMab in this example, with intact linkers having Furin cleavage sites identified by the sequence RKKR (SEQ ID NO: 26). Furin is ubiquitously expressed in most mammalian tissues and cell lines, and is capable of processing a wide range of bioactive precursor proteins in the secretory pathway, including growth factors, hormones, plasma proteins, receptors, viral envelope glycoproteins and bacterial toxins. Furin is an endoprotease localized in the Golgi complex. Furin have also been overexpressed in mammalian cells without any cell toxicity. It has also been shown that Furin can be secreted in the culture media. For the exemplary iMers, Furin cleavage is expected to occur intracellularly upon iMer assembling, but can also occur in the culture media. Furin has a stringent substrate specificity and preferentially recognizes sites that contain the sequence motif R—X—[R/K]R↓ (SEQ ID NO: 27); where X indicates any amino acid, [R/K] indicates either an arginine or a lysine, and the arrow (i) indicates the site of cleavage. Preferred recognition sequence is RKKR. Another cleavage site that can be used is TRHRQPR↓GWEQL (SEQ ID NO: 28). This former Furin sensitive spacer is derived from domain II of Pseudomonas aeruginosa toxin A. Cell lines expressing iMers can be engineered to stable overexpress a recombinant active form of the enzyme Furin. FIG. 30B provides the amino acid sequence of a representative iMer, an iMab in this example, with intact linkers having Enterokinase cleavage sites identified by the sequence GDDDK. Human Enterokinase is expressed as a linear 1019 amino acid polypeptide precursor glycoprotein. Proteolytic processing of this precursor generates the biologically active form of Enterokinase, which consists of two polypeptide chains (heavy chain and light chain) held together by a single disulfide bond, resulting in formation of a biologically active heterodimer. The heavy chain consists of 784 amino acid residues, and the light consists of 235 amino acid residues.

Several additional iMer (iMabs in this example) constructs, as described above and provided in FIGS. 21, 22 and 30-31, were generated that bound the EGF and IGF1 receptors (see for example FIG. 8A). These constructs and the parental anti-EGFR and anti-IGF1R antibodies were transiently expressed in 293 cells or CHO cells. Table 2 shows the transient expression data after 10 days for an exemplary iMab carrying the Thrombin cleavage sites, an exemplary iMab carrying the Enterokinase cleavage sites, an exemplary iMab carrying the Enterokinase cleavage sites and the Enterokinase enzyme linked at the C-terminus, and an exemplary iMab carrying the Furin-cleavage site. These expression data were determined using a protein A binding method as described elsewhere in this application.

TABLE 2
Transient expression of alternative protease constructs

Construct name

| | |
|---|---|
| | Transient expression in 293 |
| | cells after 10 days (mg/L) |
| iMab-EI with Thrombin cleavage sites | 10 |
| iMab-EI with Enterokinase cleavage sites | 11 |
| iMab-EI with Enterokinase cleavage sites | 2 |
| and linked Enterokinase enzyme | |
| | Transient expression in CHO |
| | cells after 10 days (mg/L) |
| iMab-EI-with-Furin-cleavage site | 45 |
| Furin endogenous expressed | |

Exemplary iMabs targeting EGFR and IGF1R (also referred to herein as iMab-EI), with linkers containing Enterokinase recognition sequences with or without the human Enterokinase enzyme or with linkers containing Thrombin recognitions sequences and the parental antibodies were expressed in 293 cells and purified from the culture supernatant using protein A chromatography. FIG. 31 shows the SDS-PAGE analysis in reducing condition for these constructs. The iMer-EI having linkers carrying Enterokinase recognition sequences, and the human Enterokinase enzyme as shown in FIG. 21, is secreted into the culture media as processed molecules with most of the interconnecting linker removed (lane 2). In addition, the The iMer-EI having linkers carrying Enterokinase recognition sequences without the enzyme also shows some processing (lane 3) indicating that a low level of Enterokinase activity is also present in the 293 cells used to express the constructs. In constrast the The iMer-EI having linkers carrying Thrombin recognition sequences were not cleaved (lane 7) until treated with exogenous Thrombin (lane 6). Upon cleavage all the constructs had a migration pattern that indicated both parental arms (lanes 4 and 5) where present.

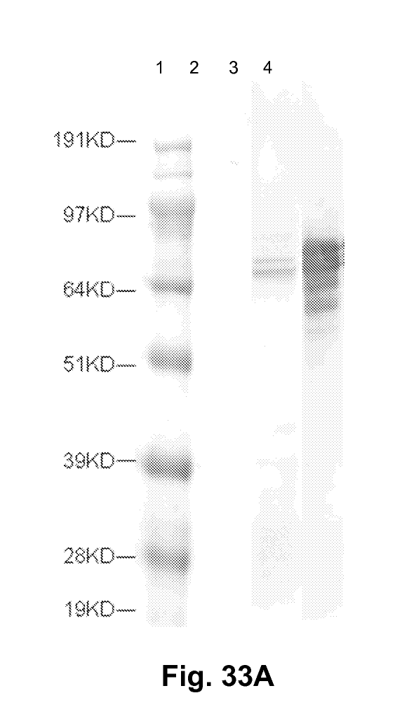

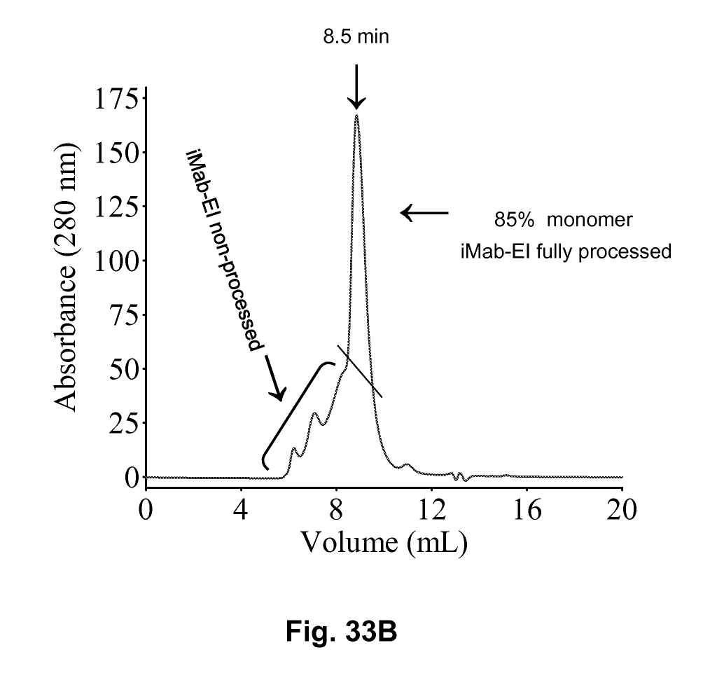

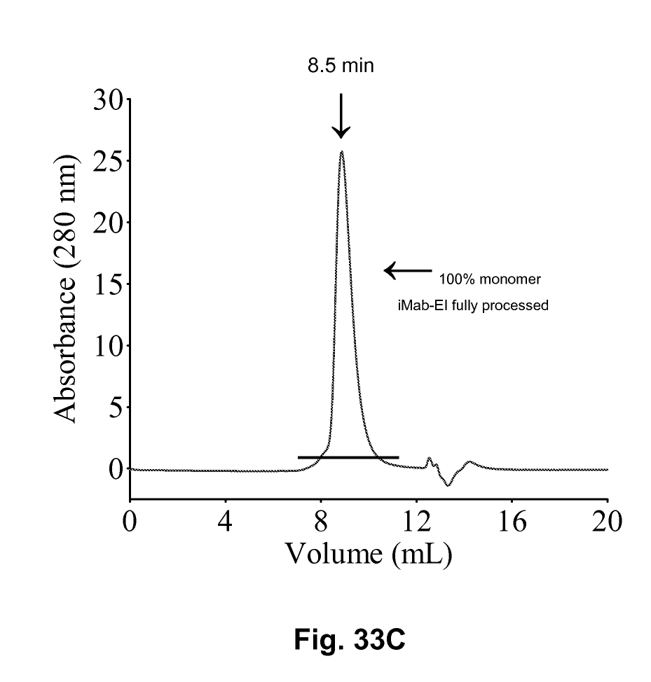

Western blot analysis of whole CHO cells probed with anti-Furin antibody (R&D System catalog number AF1503) demonstrate that CHO cells express intracellular endogenous Furin (FIG. 33A). An exemplary iMab-EI, with a Furin cleavage sites as shown in FIG. 30A was transiently expressed in CHO cells and purified from the culture supernatant using protein A essentially as described in Example 1. The expression level of this construct is shown in Table 2. SEC-HPLC analysis of the purified material was performed essentially as described in Example 2. As shown in FIG. 33B, 85% the iMab-EI with a Furin cleavage site is processed in the CHO cells and has the expected migration time of ˜8.5 minutes. As shown in FIG. 33C, fully processed iMab-EI with the Furin cleavage sites can be purified to homogeneity. These studies demonstrate that when an iMab that caries the Furin cleavage sites is expressed in cells that endogenous express Furin (such as the CHO cells) the iMab-EI is nearly fully processed and can readily be purified to homogeneity without the use of exogenous protease.

### Example 14

### iMer Alternative Fc Constructs

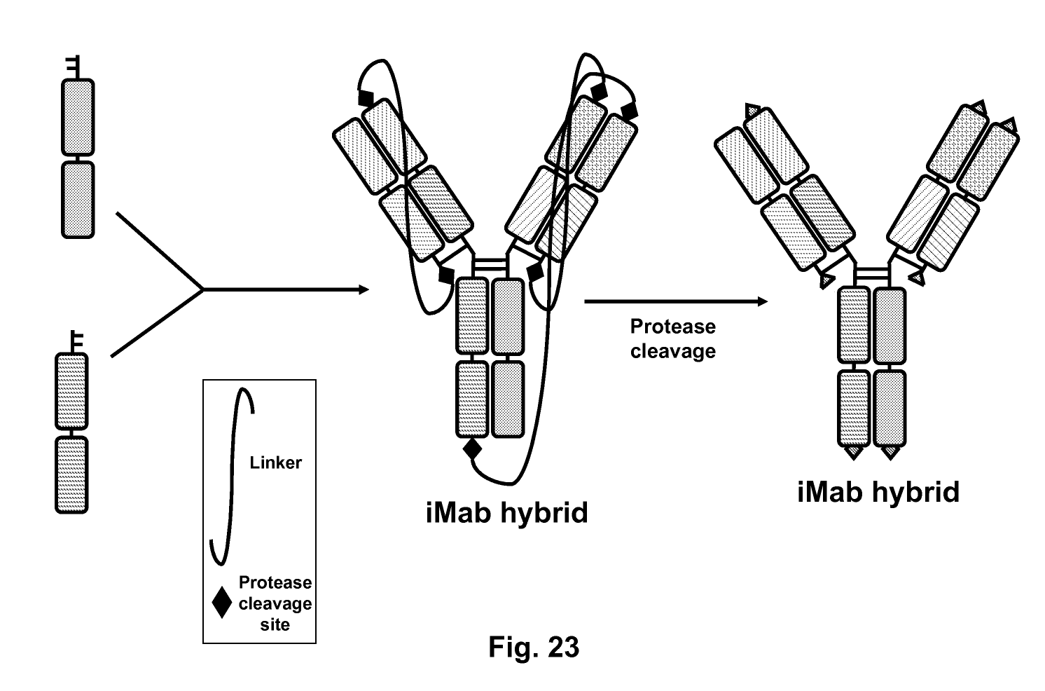

FIG. 23 provides a schematic representation of an iMer construct, an iMab in this example, that comprises distinct Fc domains (e.g., Fc domains from different IgG isotypes and/or different Fc chimeras) which is expressed as a single contiguous polypeptide chain. Also contemplated are iMers which comprise Fc domains differentially engineered with mutations to: promote and/or maintain heterodimerization (e.g., chimeric mutations, complementary mutations, lock and dock mutations, knob into hole mutations, etc.); alter half-life (e.g., enhance FcRn binding); modulate effector function (e.g., enhance ADCC); and alter stability (e.g., prevent IgG4 arm exchange). It is further contemplated that iMers may comprise only a portion of a constant region to promote and/or maintain dimerization. For example, FIG. 24 provides a schematic representation of iMer constructs that comprise only the CH3 portion of an Fc domain. These alternative Fc constructs may also be introduced into the alternative iMer constructs described below and represented in FIGS. 25-29.

### Example 15

### Alternative iMer Constructs

FIG. 25 provides schematic representations of a number of non-limiting alternative iMer constructs that can be engineered and expressed as single contiguous polypeptide chains. iMer constructs may incorporate a second polypeptide chain which is expressed separately and associates with the polypeptide portions expressed as a single contiguous polypeptide chain, see for example iMer-3n, iMer-4-n, iMer-5n and iMer-6n. FIG. 26 depicts the same iMer constructs after protease removal of the linkers. In other alternative iMer constructs, the immunoglobulin dimerization domain may be truncated and/or replaced with a non-immunoglobulin dimerization domains (see, Examples 14 and 17). Furthermore, iMers may incorporate additional functional domains (e.g. antigen binding domain, ligand binding domain, etc) in the single contiguous polypeptide chain and/or in a separate chain that associates with the polypeptide portions expressed as a single contiguous polypeptide chain.

### Example 16

### Alternative Constructs

FIGS. 27 and 28 provide schematic representations of iMab constructs having a Dual (FIG. 27) or Triple (FIG. 28) Fab domain. The left hand panels depict the constructs with the linkers, while the right hand panels depict the same constructs after protease removal of the linkers.

### Example 17

### Non-Ig Dimerization Domains

FIG. 29 provides non-limiting schematic representations of iMer constructs engineered with non-immunoglobulin dimerization domains. A non-immunoglobulin dimerization domain is another example of an Interaction Domain (ID). Dimerization motifs which can be incorporated into iMers include, but are not limited to, coil-coil homodimerization motifs such as Geminin, HIV major homology region, Saccharomyces cerevisiae Sir4p, transcription factors, zinc-finger domains, viral coat proteins, p53, and leucine-zippers.

While specific aspects of the subject disclosure have been discussed, the above specification is illustrative and not restrictive. Many variations of the disclosure will become apparent to those skilled in the art upon review of this specification and the claims below. For example, all the techniques and apparatus described above may be used in various combinations. The full scope of the disclosure should be determined by reference to the claims, along with their full scope of equivalents, and the specification, along with such variations.

### 9. INCORPORATION BY REFERENCE

All publications and patents mentioned herein are hereby incorporated by reference in their entirety as if each individual publication or patent was specifically and individually indicated to be incorporated by reference. In addition, U.S. Provisional Patent Application No. 61/504,741 filed Jul. 6, 2011 is incorporated by reference in its entirety for all purposes.

1. A nucleic acid molecule encoding a contiguous, multimeric polypeptide comprising at least two subunits, each of which includes at least a functional domain (FD) and an interaction domain (ID), wherein the nucleic acid molecule comprises:
i) a nucleic acid portion comprising a nucleotide sequence that encodes a functional domain (FD1) that binds to a first binding site;
ii) a nucleic acid portion comprising a nucleotide sequence that encodes an interaction domain (ID1);
iii) a nucleic acid portion comprising a nucleotide sequence that encodes a functional domain (FD2) that binds to a second binding site; and
iv) a nucleic acid portion comprising a nucleotide sequence that encodes an interaction domain (ID2);
wherein the nucleic acid molecule further comprises at least one nucleic acid portion comprising a nucleotide sequence encoding a polypeptide linker, which polypeptide linker includes at least one protease cleavage site;
wherein ID1 and ID2 are capable of associating with each other, and wherein the contiguous, multimeric polypeptide is multispecific.

2-4. (canceled)

5. The nucleic acid molecule of claim 1, wherein
the nucleic acid portion comprising the nucleotide sequence that encodes the ID1 and the nucleic acid portion comprising the nucleotide sequence that encodes the ID2 each comprises a nucleic acid segment comprising a nucleotide sequence that encodes a coil-coil dimerization motif; or
the nucleic acid portion comprising the nucleotide sequence that encodes the ID1 comprises a nucleic acid segment comprising a nucleotide sequence that encodes a heavy constant domain 1 and the nucleic acid portion comprising the nucleotide sequence that encodes the ID2 comprises a nucleic acid segment comprising a nucleotide sequence that encodes a heavy constant domain 2.

6. (canceled)

7. The nucleic acid molecule of claim 1, wherein
the nucleic acid portion comprising the nucleotide sequence that encodes the FD1 and/or FD2 comprises a nucleic acid segment comprising a nucleotide sequence that encodes an antigen binding portion of an antibody selected from the group consisting of: an scFv, a tandem scFv, a diabody, a triabody and a Fab domain.

8-10. (canceled)

11. The nucleic acid molecule of claim 1, wherein the nucleic acid portion comprising the nucleotide sequence that encodes FD1 and/or FD2 comprises a nucleic acid segment comprising a nucleotide sequence that encodes an antibody mimetic.

12. A nucleic acid molecule encoding a contiguous polypeptide, wherein the nucleic acid molecule comprises:
i) a nucleic acid segment comprising a nucleotide sequence that encodes an antibody light chain comprising a variable domain (VL1) and a light constant domain 1;
ii) a nucleic acid segment comprising a nucleotide sequence that encodes a first polypeptide linker;
iii) a nucleic acid segment comprising a nucleotide sequence that encodes an antibody heavy chain comprising a variable domain (VH1) and a heavy constant domain 1;
iv) a nucleic acid segment comprising a nucleotide sequence that encodes a second polypeptide linker;
v) a nucleic acid segment comprising a nucleotide sequence that encodes an antibody light chain comprising a variable domain (VL2) and a light constant domain 2;
vi) a nucleic acid segment comprising a nucleotide sequence that encodes a third polypeptide linker; and
vii) a nucleic acid segment comprising a nucleotide sequence that encodes an antibody heavy chain comprising a variable domain (VH2) and a heavy constant domain 2;
wherein the contiguous polypeptide is a multispecific polypeptide, and wherein each of the three polypeptide linkers comprises at least one protease cleavage site.

13. The nucleic acid molecule of claim 12, wherein VL1 and VL2 are not the same antibody light chain variable domain and/or VH1 and VH2 are not the same antibody heavy chain variable domain.

14. The nucleic acid molecule of claim 5, wherein the heavy constant domain 1 comprises one or more of a C_{H}1, C_{H}2, and C_{H}3 regions, and wherein the heavy constant domain 2 comprises one or more of a C_{H}1, C_{H}2, and C_{H}3 regions.

15. The nucleic acid molecule of claim 14, wherein the heavy constant domain 1 further comprises a hinge region, and wherein the heavy constant domain 2 further comprises a hinge region.

16. The nucleic acid molecule of claim 1, wherein the nucleic acid molecule comprises at least two nucleic acid segments each of which comprises a nucleotide sequence that encodes a polypeptide linker.

17. The nucleic acid molecule of any of claim 1, wherein at least one of the polypeptide linkers comprises two protease cleavage sites.

18. The nucleic acid molecule of claim 16, wherein at least one of the polypeptide linkers is different.

19. An expression vector comprising the nucleic acid molecule of any of claim 1 operably linked to a promoter.

20. A host cell comprising the expression vector of claim 19, and which host cell expresses the contiguous polypeptide.

21. A method of producing a contiguous polypeptide, comprising:
i) providing a host cell of claim 20 in a culture media suitable for growth of the cell and production of the polypeptide; and
ii) purifying the polypeptide from the cell or culture media.

22. The method of claim 21, further comprising adding, either before or after the step of purifying the polypeptide from the cell or culture media, a protease that cleaves the at least one protease cleavage site.

23-37. (canceled)

38. The nucleic acid molecule of claim 12, wherein the heavy constant domain 1 comprises a hinge, C_{H}1, C_{H}2, and C_{H}3 regions, and wherein the heavy constant domain 2 comprises a hinge, C_{H}1, C_{H}2, and C_{H}3 regions.

39. The nucleic acid molecule of any of claim 12, wherein at least one of the polypeptide linkers comprises two protease cleavage sites.

40. The nucleic acid molecule of claim 12, wherein at least one of the polypeptide linkers is different.

41. An expression vector comprising the nucleic acid molecule of claim 12 operably linked to a promoter.

42. A host cell comprising the expression vector of claim 41, and which host cell expresses the contiguous polypeptide.

43. A method of producing a contiguous polypeptide, comprising:
i) providing a host cell of claim 42 in a culture media suitable for growth of the cell and production of the polypeptide; and
ii) purifying the polypeptide from the cell or culture media.

44. The method of claim 43, further comprising adding, either before or after the step of purifying the polypeptide from the cell or culture media, a protease that cleaves the at least one protease cleavage site.

45. A polypeptide encoded by the nucleic acid molecule of claim 12.


SEQUENCE LISTING (58 sequences)

SEQ ID NO: 1 - protein, 120 aa, Artificial Sequence
  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSA

SEQ ID NO: 2 - DNA, 360 bp, Artificial Sequence
  Synthetic construct
caagttcaacttcaagaatctggtcctggtcttgttaaaccttctgaaactctttctcttacttgtactgtttctggtggttctgtttcttctggtgattattattggacttggattcgtcaatctcctggtaaaggtcttgaatggattggtcatatttattattctggtaatactaattataatccttctcttaaatctcgtcttactatttctattgatacttctaaaactcaattttctcttaaactttcttctgttactgctgctgatactgctatttattattgtgttcgtgatcgtgttactggtgcttttgatatttggggtcaaggtactatggttactgtttcttctgct

SEQ ID NO: 3 - protein, 107 aa, Artificial Sequence [sequence omitted]

SEQ ID NO: 4 - DNA, 321 bp, Artificial Sequence
  Synthetic construct
gatattcaaatgactcaatctccttcttctctttctgcttctgttggtgatcgtgttactattacttgtcaagcttctcaagatatttctaattatcttaattggtatcaacaaaaacctggtaaagctcctaaacttcttatttatgatgcttctaatcttgaaactggtgttccttctcgtttttctggttctggttctggtactgattttacttttactatttcttctcttcaacctgaagatattgctacttatttttgtcaacattttgatcatcttcctcttgcttttggtggtggtactaaagttgaaattaaa

SEQ ID NO: 5 - protein, 120 aa, Artificial Sequence
  Synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAA

SEQ ID NO: 6 - DNA, 360 bp, Artificial Sequence
  Synthetic construct
caagttcaacttaaacaatctggtcctggtcttgttcaaccttctcaatctctttctattacttgtactgtttctggtttttctcttactaattatggtgttcattgggttcgtcaatctcctggtaaaggtcttgaatggcttggtgttatttggtctggtggtaatactgattataatactccttttacttctcgtctttctattaataaagataattctaaatctcaagttttttttaaaatgaattctcttcaatctaatgatactgctatttattattgtgctcgtgctcttacttattatgattatgaatttgcttattggggtcaaggtactcttgttactgtttctgctgct

SEQ ID NO: 7 - protein, 107 aa, Artificial Sequence
  Synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 8 - DNA, 321 bp, Artificial Sequence
  Synthetic construct
gatattcttcttactcaatctcctgttattctttctgtttctcctggtgaacgtgtttctttttcttgtcgtgcttctcaatctattggtactaatattcattggtatcaacaacgtactaatggttctcctcgtcttcttattaaatatgcttctgaatctatttctggtattccttctcgtttttctggttctggttctggtactgattttactctttctattaattctgttgaatctgaagatattgctgattattattgtcaacaaaataataattggcctactacttttggtgctggtactaaacttgaacttaaa

SEQ ID NO: 9 - protein, 123 aa, Artificial Sequence [sequence omitted]

SEQ ID NO: 10 - DNA, 369 bp, Artificial Sequence
  Synthetic construct
caagttcaacttcaacaatctggtgctgaagttaaaaaacctggttcttctgttaaagtttcttgtaaagcttctggttatacttttactaattattatatttattgggttcgtcaagctcctggtcaaggtcttgaatggattggtggtattaatcctacttctggtggttctaattttaatgaaaaatttaaaactcgtgttactattactgctgatgaatcttctactactgcttatatggaactttcttctcttcgttctgaagatactgctttttatttttgtactcgtcaaggtctttggtttgattctgatggtcgtggttttgatttttggggtcaaggtactactgttactgtttcttct

SEQ ID NO: 11 - protein, 113 aa, Artificial Sequence
  Synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITR

SEQ ID NO: 12 - DNA, 339 bp, Artificial Sequence
  Synthetic construct
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgt

SEQ ID NO: 13 - protein, 125 aa, Artificial Sequence [sequence omitted]

SEQ ID NO: 14 - DNA, 375 bp, Artificial Sequence
  Synthetic construct
gaagttcaacttcttgaatctggtggtggtcttgttcaacctggtggttctcttcgtctttcttgtactgcttctggttttactttttcttcttatgctatgaattgggttcgtcaagctcctggtaaaggtcttgaatgggtttctgctatttctggttctggtggtactactttttatgctgattctgttaaaggtcgttttactatttctcgtgataattctcgtactactctttatcttcaaatgaattctcttcgtgctgaagatactgctgtttattattgtgctaaagatcttggttggtctgattcttattattattattatggtatggatgtttggggtcaaggtactactgttactgtttcttct

SEQ ID NO: 15 - protein, 107 aa, Artificial Sequence [sequence omitted]

SEQ ID NO: 16 - DNA, 321 bp, Artificial Sequence
  Synthetic construct
gatattcaaatgactcaatttccttcttctctttctgcttctgttggtgatcgtgttactattacttgtcgtgcttctcaaggtattcgtaatgatcttggttggtatcaacaaaaacctggtaaagctcctaaacgtcttatttatgctgcttctcgtcttcatcgtggtgttccttctcgtttttctggttctggttctggtactgaatttactcttactatttcttctcttcaacctgaagattttgctacttattattgtcttcaacataattcttatccttgttcttttggtcaaggtactaaacttgaaattaaa

SEQ ID NO: 17 - protein, 130 aa, Artificial Sequence
  Synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 18 - DNA, 390 bp, Artificial Sequence
  Synthetic construct
gaagttcaacttgttcaatctggtgctgaagttaaaaaacctggttcttctgttaaagtttcttgtaaagcttctggtggtactttttcttcttatgctatttcttgggttcgtcaagctcctggtcaaggtcttgaatggatgggtggtattattcctatttttggtactgctaattatgctcaaaaatttcaaggtcgtgttactattactgctgataaatctacttctactgcttatatggaactttcttctcttcgttctgaagatactgctgtttattattgtgctcgtgctcctcttcgttttcttgaatggtctactcaagatcattattattattattatatggatgtttggggtaaaggtactactgttactgtttcttct

SEQ ID NO: 19 - protein, 114 aa, Artificial Sequence
  Synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAA

SEQ ID NO: 20 - DNA, 342 bp, Artificial Sequence
  Synthetic construct
tcttctgaacttactcaagatcctgctgtttctgttgctcttggtcaaactgttcgtattacttgtcaaggtgattctcttcgttcttattatgctacttggtatcaacaaaaacctggtcaagctcctattcttgttatttatggtgaaaataaacgtccttctggtattcctgatcgtttttctggttcttcttctggtaatactgcttctcttactattactggtgctcaagctgaagatgaagctgattattattgtaaatctcgtgatggttctggtcaacatcttgtttttggtggtggtactaaacttactgttcttggtcaacctaaagctgct

SEQ ID NO: 21 - protein, 118 aa, Artificial Sequence
  Synthetic construct
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSSA

SEQ ID NO: 22 - DNA, 354 bp, Artificial Sequence
  Synthetic construct
caagttcaacttcaagaatctggtcctggtcttgttaaaccttctgaaactctttctcttacttgtactgtttctggttattctatttctggtggttatctttggaattggattcgtcaacctcctggtaaaggtcttgaatggattggttatatttcttatgatggtactaataattataaaccttctcttaaagatcgtgttactatttctgttgatacttctaaaaatcaattttctcttaaactttcttctgttactgctgctgatactgctgtttattattgtgctcgttatggtcgtgttttttttgattattggggtcaaggtactcttgttactgtttcttctgct

SEQ ID NO: 23 - protein, 112 aa, Artificial Sequence [sequence omitted]

SEQ ID NO: 24 - DNA, 336 bp, Artificial Sequence
  Synthetic construct
gatattgttatgactcaatctcctctttctcttcctgttactcctggtgaacctgcttctatttcttgtcgttcttctcaatctattgttcattctaatggtaatacttatcttcaatggtatcttcaaaaacctggtcaatctcctcaacttcttatttataaagtttctaatcgtctttatggtgttcctgatcgtttttctggttctggttctggtactgattttactcttaaaatttctcgtgttgaagctgaagatgttggtgtttattattgttttcaaggttctcatgttccttggacttttggtcaaggtactaaagttgaaattaaa

SEQ ID NO: 25 - protein, 5 aa, Artificial Sequence
  Synthetic construct
GDDDK

SEQ ID NO: 26 - protein, 4 aa, Artificial Sequence
  Synthetic construct
RKKR

SEQ ID NO: 27 - protein, 5 aa, Artificial Sequence
  Synthetic construct
RXRXR

SEQ ID NO: 28 - protein, 12 aa, Artificial Sequence
  Synthetic construct
TRHRQPRGWEQL

SEQ ID NO: 29 - DNA, 633 bp, Artificial Sequence
  Synthetic construct
cgtacggtggctgcccccagcgtgttcatcttcccacccagcgacgagcagctgaagtccggcaccgccagcgtcgtgtgcctgctgaacaacttctacccccgcgaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgtggcggaggcggctctctggtgcccagaggatctgctagcggcggaggtggaagcggaggcggaggatctggtggcggaggttctggcggagggggaagtggcggtggtggatcaggcggcggaggctctatcgatggcagcgctggcggaggaagcgggggaggcggatcttctgctggtggtggcggatctggcggtggcggtagcggaggtggtggaagtgggggaggtggtagtggtggcggcggtagtggggggggaggctctggtgtacacagcctggtgcctagaggcagcggagggggcggatct

SEQ ID NO: 30 - DNA, 1485 bp, Artificial Sequence
  Synthetic construct
gccagcaccaagggcccctccgtgttccctctggcccccagcagcaagagcacatctggcggaacagccgccctgggctgcctcgtgaaggactacttccccgagcccgtgaccgtgtcctggaactctggcgccctgaccagcggcgtgcacacctttccagccgtgctgcagagcagcggcctgtactccctgtcctccgtcgtgaccgtgcccagctctagcctgggcacccagacctacatctgcaacgtgaaccacaagcccagcaacaccaaagtggacaagcgggtggaacccaagagctgcgacaagacccacacctgtcccccctgccctgcccctgaactgctgggcggacctagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaatgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtctacacactgccccccagccgggaagagatgaccaagaaccaggtgtccctgacctgtctggtcaagggcttctacccctccgatatcgccgtggaatgggagagcaacggccagcccgagaacaactacaagaccaccccccctgtgctggacagcgacggctcattcttcctgtactccaagctgaccgtggacaagagccggtggcagcagggcaacgtgttcagctgcagcgtgatgcacgaggccctgcacaaccactacacccagaagtccctgagcctgagccctggcaaaggatccgggggtggcggcagtctggtgccaaggggttcaggcgggggaggatcaggtggtggtggctcagggggtggtggttccggtggcggtggatctggggggggtggaagtggcggtggtggctctggcgggggaggcagcttaattaaagggggcggaggcagtggtggtggagggtctgggggagggggatcaggcggaggtggcctagatctgggaggcggcggttcagggggaggcggttctgggggcggaggtagcggtggtggaggcagtggtggcgggggtagtgggggtggtggatctggccggccgggtggtggtggcagtggtggtggcggctcaggtggcggaggaagtggcggcggaggcagtcacgtgggaggtggtggttctgggggtggcggaagtggtggcggtggaagcggtggtggtggtagcggagccgccgctctcgagcttgttcctaggggttcaggcggcggaggcagc

SEQ ID NO: 31 - DNA, 627 bp, Artificial Sequence
  Synthetic construct
ggccagcccaaggccgctcccagcgtgaccctgttcccaccctccagcgaggaactgcaggccaacaaagccaccctcgtgtgtctgatcagcgacttctaccctggcgccgtgacagtggcttggaaggccgacagctcccccgtgaaggccggcgtggaaaccaccacccccagcaagcagtccaacaacaaatacgccgccagcagctacctgtctctgacccccgagcagtggaagtcccaccggtcctacagctgccaggtcacacacgagggcagcaccgtggaaaagaccgtggcccccaccgagtgctcagggggaggtggcagtctggtcccaagaggaagcggagggggcggaagcggtaccggtggtggtggttcaggcggaggcggcagtggaggaggtggcagcggaggcggtggttcttcgcgaggcggagggggttcaggggggggtggtagtgggggtggtggcagcggcggtggtggatctgggggagggggctcaaagcttggaggcggtggctctggaggcggtggcagtggcgggggaggtagtggcggaggtggcggatcagcggccgccggactggtgccaagaggctccggtggcggtggatct

SEQ ID NO: 32 - DNA, 990 bp, Homo sapiens
gcctccacaaagggcccaagcgtgttccccctcgccccatcctccaagagcaccagcggaggcacagccgctctcggatgcctggtcaaagattactttcctgagcctgtcaccgtcagctggaacagcggagccctgacctctggggtgcacacattccccgctgtgctccagtcctccggcctgtatagcctgtctagcgtggtcacagtgcctagcagcagcctgggaacacagacatatatctgtaatgtcaatcacaagccctccaataccaaggtcgacaagagagtggaacctaagtcctgtgataagacacacacatgccctccctgtcccgctcccgagctgctgggaggcccttccgtgtttctgtttccacctaagcctaaggatacactcatgatctcccgcacacccgaagtcacatgtgtggtcgtggatgtgtctcacgaagatcccgaagtcaagtttaactggtatgtggatggggtcgaggtccacaacgccaagacaaaacctcgggaagaacagtataactccacatatcgcgtcgtgtctgtcctgacagtcctgcatcaggattggctcaatgggaaagaatacaaatgtaaagtctctaacaaggctctccccgctcctatcgaaaagaccatctccaaagccaaagggcagcctcgggaaccccaggtctacactctcccacctagccgcgaggaaatgacaaaaaatcaggtgtcactcacatgcctcgtcaaggggttttaccccagcgacattgccgtcgagtgggagtccaatggacagcctgaaaacaattataagaccacacctcccgtcctggactccgatggctcattttttctgtatagcaagctcaccgtcgataagtccagatggcagcagggaaatgtcttttcctgctctgtgatgcatgaagccctccataatcactatacacagaagtctctgtccctgtcccctggcaag

SEQ ID NO: 33 - protein, 209 aa, Artificial Sequence
  Synthetic construct
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLVPRGSASGGGGSGGGGSGGGGSGGGGSGGGGSGGGGSIDGSAGGGSGGGGSSAGGGGSGGGGSGGGGSGGGGSGGGGSGGGGSGVHSLVPRGSGGGGS

SEQ ID NO: 34 - protein, 495 aa, Artificial Sequence [sequence omitted]

SEQ ID NO: 35 - protein, 206 aa, Artificial Sequence
  Synthetic construct
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSLVPRGSGGGGSGTGGGGSGGGGSGGGGSGGGGSSRGGGGSGGGGSGGGGSGGGGSGGGGSKLGGGGSGGGGSGGGGSGGGGGSAAAGLVPRGSGGGGS

SEQ ID NO: 36 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 114 aa, Artificial Sequence
  Synthetic construct
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLVPR

SEQ ID NO: 38 - protein, 11 aa, Artificial Sequence
  Synthetic construct
XXGGGGSXXXX

SEQ ID NO: 39 - protein, 341 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSLVPR

SEQ ID NO: 40 - protein, 112 aa, Artificial Sequence
  Synthetic construct
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSLVPR

SEQ ID NO: 41 - protein, 205 aa, Artificial Sequence
  Synthetic construct
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSRKKRASGGGGSGGGGSGGGGSGGGGSGGGGSGGGGSIDGSAGGGSGGGGSSAGGGGSGGGGSGGGGSGGGGSGGGGSGGGGSGVHSRKKRGGGGS

SEQ ID NO: 42 - protein, 491 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSRKKRGGGGSGGGGSGGGGSGGGGSGGGGSGGGGSGGGGSLIKGGGGSGGGGSGGGGSGGGGLDLGGGGSGGGGSGGGGSGGGGSGGGGSGGGGSGRPGGGGSGGGGSGGGGSGGGGSHVGGGGSGGGGSGGGGSGGGGSGAAALERKKRGGGGS

SEQ ID NO: 43 - protein, 202 aa, Artificial Sequence
  Synthetic construct
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSRKKRGGGGSGTGGGGSGGGGSGGGGSGGGGSSRGGGGSGGGGSGGGGSGGGGSGGGGSKLGGGGSGGGGSGGGGSGGGGGSAAAGRKKRGGGGS

SEQ ID NO: 44 - protein, 200 aa, Artificial Sequence
  Synthetic construct
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDDDDKASGGGGSGGGGSGGGGSGGGGSGGGGSGGGGSIDGSAGGGSGGGGSSAGGGGSGGGGSGGGGSGGGGSGGGGSGGGGSGVHSGDDDDK

SEQ ID NO: 45 - protein, 490 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGDDDDKGGGGSGGGGSGGGGSGGGGSGGGGSGGGGSGGGGSLIKGGGGSGGGGSGGGGSGGGGLDLGGGGSGGGGSGGGGSGGGGSGGGGSGGGGSGRPGGGGSGGGGSGGGGSGGGGSHVGGGGSGGGGSGGGGSGGGGSGAAALEGDDDDK

SEQ ID NO: 46 - protein, 196 aa, Artificial Sequence
  Synthetic construct
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGDDDDKGGGGSGTGGGGSGGGGSGGGGSGGGGSSRGGGGSGGGGSGGGGSGGGGSGGGGSKLGGGGSGGGGSGGGGSGGGGGSAAAGGDDDDK

SEQ ID NO: 47 - protein, 581 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGDDDDKGGGGGIVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPISLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLH

SEQ ID NO: 48 - protein, 6 aa, Artificial Sequence
  Synthetic construct
LVPRGS

SEQ ID NO: 49 - protein, 104 aa, Artificial Sequence
  sythetic construct
GGGGSLVPRGSASGGGGSGGGGSGGGGSGGGGSGGGGSGGGGSIDGSAGGGSGGGGSSAGGGGSGGGGSGGGGSGGGGSGGGGSGGGGSGVHSLVPRGSGGGGS

SEQ ID NO: 50 - protein, 165 aa, Artificial Sequence
  sythetic construct
GSGGGGSLVPRGSGGGGSGGGGSGGGGSGGGGSGGGGSGGGGSGGGGSLIKGGGGSGGGGSGGGGSGGGGLDLGGGGSGGGGSGGGGSGGGGSGGGGSGGGGSGRPGGGGSGGGGSGGGGSGGGGSHVGGGGSGGGGSGGGGSGGGGSGAAALELVPRGSGGGGS

SEQ ID NO: 51 - protein, 103 aa, Artificial Sequence
  sythetic construct
GGGGSLVPRGSGGGGSGTGGGGSGGGGSGGGGSGGGGSSRGGGGSGGGGSGGGGSGGGGSGGGGSKLGGGGSGGGGSGGGGSGGGGGSAAAGLVPRGSGGGGS

SEQ ID NO: 52 - protein, 100 aa, Artificial Sequence
  sythetic construct
GGGGSRKKRASGGGGSGGGGSGGGGSGGGGSGGGGSGGGGSIDGSAGGGSGGGGSSAGGGGSGGGGSGGGGSGGGGSGGGGSGGGGSGVHSRKKRGGGGS

SEQ ID NO: 53 - protein, 161 aa, Artificial Sequence
  sythetic construct
GSGGGGSRKKRGGGGSGGGGSGGGGSGGGGSGGGGSGGGGSGGGGSLIKGGGGSGGGGSGGGGSGGGGLDLGGGGSGGGGSGGGGSGGGGSGGGGSGGGGSGRPGGGGSGGGGSGGGGSGGGGSHVGGGGSGGGGSGGGGSGGGGSGAAALERKKRGGGGS

SEQ ID NO: 54 - protein, 99 aa, Artificial Sequence
  sythetic construct
GGGGSRKKRGGGGSGTGGGGSGGGGSGGGGSGGGGSSRGGGGSGGGGSGGGGSGGGGSGGGGSKLGGGGSGGGGSGGGGSGGGGGSAAAGRKKRGGGGS

SEQ ID NO: 55 - protein, 95 aa, Artificial Sequence
  sythetic construct
GGDDDDKASGGGGSGGGGSGGGGSGGGGSGGGGSGGGGSIDGSAGGGSGGGGSSAGGGGSGGGGSGGGGSGGGGSGGGGSGGGGSGVHSGDDDDK

SEQ ID NO: 56 - protein, 160 aa, Artificial Sequence
  sythetic construct
GSGGGGSGDDDDKGGGGSGGGGSGGGGSGGGGSGGGGSGGGGSGGGGSLIKGGGGSGGGGSGGGGSGGGGLDLGGGGSGGGGSGGGGSGGGGSGGGGSGGGGSGRPGGGGSGGGGSGGGGSGGGGSHVGGGGSGGGGSGGGGSGGGGSGAAALEGDDDDK

SEQ ID NO: 57 - protein, 93 aa, Artificial Sequence
  sythetic construct
GDDDDKGGGGSGTGGGGSGGGGSGGGGSGGGGSSRGGGGSGGGGSGGGGSGGGGSGGGGSKLGGGGSGGGGSGGGGSGGGGGSAAAGGDDDDK

SEQ ID NO: 58 - protein, 251 aa, Artificial Sequence
  sythetic construct
GGGGGGDDDDKGGGGGIVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPISLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLH